(19) United States
(12) Patent Application Publication
(10) Pub. No.: US 2005/0037350 A1
(43) Pub. Date: Feb. 17, 2005

(54) Nucleic acid-based method for tree phenotype prediction: dna markers for fibre coarseness, microfibril angle, pulp strength and yield, lignin content, pitch propensity and calcium accumulation determinants

(76) Inventors: Simon Potter, Vancouver (CA); Paul Andrew Watson, Vancouver (CA)

Correspondence Address: OGILVY RENAULT, 1981 MCGILL COLLEGE AVENUE, SUITE 1600, MONTREAL, QC, H3A2Y3, CA

(21) Appl. No.: 10/481,697

(22) PCT Filed: Jun. 25, 2002
(86) PCT No.: PCT/CA02/00942

Related U.S. Application Data
(60) Provisional application No. 60/300,073, filed on Jun. 25, 2001.
(60) Provisional application No. 60/322,421, filed on Sep. 17, 2001.

(51) Int. Cl. C12Q 1/68; C12P 19/34

(52) U.S. Cl. 435/6; 435/91.2

(57) ABSTRACT

The present invention relates to methods for identifying individual trees having a superior phenotype and more particularly to molecular markers and/or quantitative trait loci (QTL) which can be used to identify individual trees having a superior phenotype. The molecular markers and/or QTL comprise restriction fragment length polymorphism pattern or PCR-fingerprint. The molecular markers and/or QTL can be used for the development of marker-assisted breeding, rapid assessment techniques or to identify orthologous functional genes by sequence homology in trees of different a genus and/or species. DNA markers are e.g. for fibre coarseness, microfibril angle, pulpstrength and yield, lignin content, pitch propensity and calcium accumulation determinants.

### BACKGROUND OF THE INVENTION

a) Field of the Invention

This invention is in the fields of tree improvement, forestry and pulp and paper evaluation technology. This invention allows for an enhanced selection efficiency for given trees from both natural and plantation populations with specific fibre and wood quality properties for value-added pulp and paper product lines.

b) Description of Prior Art

The utilization of species of the Populus genus of forest trees, particularly aspen and cottonwoods, as the cornerstone for the development of short-rotation intensive culture (SRIC) sustainable plantation forestry in the northern hemisphere has been promoted for a number of reasons, including greenhouse gas amelioration and phytoremediation. The primary driving force behind the implementation of SRIC Populus plantations, however, is their potential to alleviate the shortfall in world fibre supplies projected for 2010 (Wilson, R. A. and Ward, M. R. “Biotechnology holds the key as world demand soars,” Pulp Pap. Int. 38 #8, 41-43 (1996)). This threat has provided an impetus for the examination of alternative fibre sources. Many non-wood sources have been characterized (Watson, P. and Garner, A. “The opportunities for producing pulp from agricultural residues in Alberta. A review of non-wood pulping technologies,” Alberta Econ. Dev. & Tourism publication (1996); Watson, P. A., Bicho, P. and Stumborg, M. A. Pulp Pap. Can. 99(12), 146-149 (1998)) but the most logical and industrially expedient solution to the problem is likely to lie in fast-growing hardwood tree species. In the Southern hemisphere (and some parts of Europe), eucalyptus species are the hardwood of choice being prized for their growth rate, inherent adaptability and excellent papermaking properties [Kibblewhite, R. P. and McKenzie, C. J. “Kraft fibre property variation among 29 trees of 15 year old E. fastigata and comparison with E. nitens,” Appita J. 52, 218-225 (1999). Cotterill, P. and Macrae, S. “Improving Eucalyptus pulp and paper quality using genetic selection and good organization,” Tappi J. 80 (6), 82-89 (1997)]. In the Northern hemisphere members of the genus Populus (including poplars and aspen) represent a similar opportunity having high growth rates—up to 30 m³/ha/yr [Cisneros, H., Belanger, L., Gee, W. Y., Watson, P. A., and Hatton, J. V. “Wood and fibre properties of hybrid poplars from southern British Columbia,” TAPPI J. 83(7) (2000)] in cold climates—producing pulps of high natural brightness (Tice, B. “A tale of two tree farms,” Logging and sawmill Journal, 38-42 (June 1998)) and a wide range of fibre, pulping and pulp properties (Smook, G. A. Handbook for Pulp and Paper Technologists (2^{nd} ed.). Angus Wilde Publications. ISBN 0-9694628-1-6 (1992)).

Advantageously, poplars are unique in the additional potential they offer for genetic improvement of wood quality traits. Hybrid poplars are particularly well suited to genetic mapping studies as they are readily amenable to interspecies crosses, the progeny grow rapidly, and they have a relatively small genome. These advantages imply that the identification and manipulation of genetic control elements in poplars will be at least twice as easy as in rival fast-growing species such as eucalyptus, and forty times easier than in radiata pine. Information generated by studies of this kind is extremely valuable for a number of reasons. Genetic control elements can be used to both rapidly and easily identify superior clonal material in natural populations and to screen such material for plantation establishment. Additionally, knowledge of the genetic structure of superior clones will open the door to transgenic manipulation to produce “ideal” trees (ideotypes) for specific end-product applications.

In a previous study on a genetically well-characterized three-generation family of hybrid poplars (Populus trichocarpa X Populus deltoides—Family 331) developed by the University of Washington, this potential was assessed and exploited [PD4145]. Quantitative trait loci (QTL—genomic regions containing genes involved in the control of continuously variable traits) for wood and fibre quality traits were determined.

Regions of DNA which contain multiple genes affecting the same physical trait are known as quantitative trait loci (QTLs). These regions are detected using genetic marker technology and their presence or absence can be statistically correlated in tree populations with the magnitude of a particular physiological trait, such as fibre length. This statistical association is based on the technique of multiple simultaneous linear regressions of trait data with genetic marker presence/absence data using computer software. In this way, genetic maps can be “scanned” for groups of markers which correlate with the trait of interest—this group of markers is then classified as bounding a QTL partially controlling that trait (in other words, the markers are not the genes involved in the control of the trait, but those genes exist within the region of DNA bounded by the markers—this method is known as interval mapping). The degree of association between the markers and the trait can be used to estimate the “strength” of the QTL, i.e., the percentage of the trait variance which that particular QTL can account for.

As an extension of, and complement to, this previous study, additional phenotypic information has been gathered for the same family grown at three separate sites. In this case, the industrially relevant traits examined were:
fibre coarseness
microfibril angle
kraft pulp yield
lignin content
macerated fibre yield
pulp properties including strength and air resistance
kraft pulping H-factor
specific refining energy
wood extractive compounds content
calcium salt accumulation

The first four properties examined, fibre coarseness, microfibril angle, pulp yield and lignin content, are all critical pulp and papermaking parameters. The properties of a sheet of paper are dependent on the structural characteristics of the fibres which compose that sheet, the two most important characteristics being the length of the fibres and their coarseness (a weight to length measure). Length is required for strength properties, particularly so for hardwood species as longer-fibred hardwood pulps can be used to reduce the expensive softwood component of certain papermaking furnishes. In softwoods, increasing fibre length can actually be problematic as excessively long fibres are prone to flocculation. Coarseness is often (but not always, c.f. red and sugar maple) a reasonable indicator of the thickness of the fibre cell wall. Wall thickness determines whether the fibres will collapse to readily form flat ribbons, giving paper sheets a smooth surface, or be less uncollapsible providing sheet bulk and absorbancy. Consequently, coarser, generally thicker-walled, fibres (e.g. Douglas fir) resist collapse and produce open, absorbent, bulky sheets with low burst/tensile strength and high tear strength.

The structural framework of the cell wall of fibres is primarily provided by cellulose microfibrils in the thickest S2 layer, cemented together with lignin. The lignin binds the microfibrils and prevents their lateral buckling under load. The parameter microfibril angle indicates the angle to the longitudinal axis of the fibre at which the microfibrils are wound around the cell in a spiral formation. The smaller the angle, the steeper the spiral (in general, microfibril angle is at its highest near the pith, decreases through the juvenile wood core and then reaches a stable level in the mature wood). Microfibril angle has a major effect on the physical strength of the fibre as it dictates the amount of tension that may be directed axially along the microfibril. The steeper the angle, the stronger the fibre and the higher the tensile modulus. In this capacity therefore, microfibril angle is a critical strength parameter for both pulp and paper and solid wood applications of forest species.

Pulp yield is a measure of the amount of fibre recovered from an initial charge of wood. A great deal of chemical engineering effort is routinely expended to achieve process improvements in yield of the order of 0.5-1.0%. (e.g. polysulfide process). As wood quality databases become gradually more comprehensive, it is clear that both inter- and intra-species variability for this parameter can vastly outweigh such a change. Indeed, recent research has suggested that choosing one aspen (Populus tremuloides) clone over another of the same species for pulping can result in a yield improvement of 4-6%, at a given kappa number. The efficiency of the pulping process, and a number of subsequent papermaking parameters, are critically dependent on the amount and chemical composition of the lignin polymer found in the wood. Normal softwood lignin is mainly composed of guaiacylpropane subunits which are difficult to remove via conventional processes. By contrast, hardwood lignin is composed of both guaiacyl- and syringylpropane units, in which the ratio of the two phenylpropanes varies between species [Higuchi, T. “Biochemistry and Molecular Biology of Wood, ” Springer-Verlag. ISBN 3-540-61367-6 (1997)]. If the genetic control of the lignin biosynthetic pathway can be determined, it may be possible to assess softwood populations for clones with hardwood-like lignin or to produce more syringyl residues in softwood lignin. Transgenic manipulation will also be possible and, indeed, several research groups are already manipulating some of the control enzymes of the lignin biosynthetic pathway with varying results (Lee, D. and Douglas, C. J. “Manipulation of plant gene expression using antisense RNA” in Plant Biochemistry and Molecular Biology (Daschek, W. ed.). CRC Press (1996), Hauffe, K. D., Lee, S. P., Subramaniam, R., and Douglas, C. J. “Combinatorial interactions between positive and negative cis-acting elements control spacial patterns of 4CL1 expression in transgenic tobacco,” Plant J. 4, 235-253 (1993)).

Specific extractives of wood are well known to cause adverse effects on various aspects of pulp and papermaking, specifically pitch deposition (Sithole, B. and Allen, L. H. “The effects of wood extractives on system closure,” presented at the 2000 TAPPSA conference in Durban South Africa (October, 2000). Lorencak, P., Baumann, P., and Hughes, D. “Deaeration in high temperature systems” Paper Tech. 38(9), 25-28 (1997). Allen, L. H. “Mechanisms and control of pitch deposition in newsprint mills,” Tappi J. 63(2), 81-87 (1980)] and effluent toxicity, particularly for mechanical pulping operations. It has been estimated that pitch deposition problems (such as dispersed wood resin, metal soaps, wood resin component polymerization and surface active agent foaming) cost the Canadian industry several hundred million dollars annually. These extractive effects in open systems are already disproportionate to their concentration (extractives comprise˜1-5% of the weight of wood) and it is anticipated that the problems will be exacerbated by progress towards mill system closure. For species used in mechanical pulping, such as aspen and related species, there are additional problems with pulp brightening caused by high extractives content [Ekman, R., Eckerman, C., and Holmbom, B. “Studies on the behaviour of extractives in mechanical pulp suspensions,” Nord. Pulp Pap. Res. J., 5, 96-102 (1990)).

A number of research groups have previously noted that certain poplar species have an inherent tendency to accumulate mineral deposits, particularly calcium salt crystals in their wood (Muhammad, A. F. and Micko, M. M. “Accumulation of calcium crystals in the decayed wood of aspen attacked by Fomes igniarius,” IAWA B. 5, 237-241 (1984), Janin, G. and Clement, A. “Calcium carbonate crystals in the wood of poplars. Effect on the distribution of mineral ions related to the formation of heartwood,” Ann. Sci. For. 29, 67-105 (1972)). Evidence described in these papers suggests that these crystals do not represent abnormalities but rather are consistently present in some Populus lineages (particularly the sections Aigeros and Tacamahaca). The crystals were found to accumulate in the stem, branches, roots and within vessels and fibres frequently occluding them completely. This paper reports the confirmation of these findings using the well-characterized hybrid poplar family and documents the effects of these crystals on the pulp properties of the hybrid family.

### SUMMARY OF THE INVENTION

One aim of the present invention is to provide a method for identifying individual trees having a superior phenotype.

In accordance with the present invention there is provided a method of identifying a gene in tree of a second genus and/or species capable of expressing desired biological and/or biochemical phenotypes, said second tree genus and/or species being of different genus and/or species than a first tree species, comprising the steps of (a) obtaining a nucleic acid sample from tree of a first genus and/or species and/or hybrid thereof; (b) obtaining either a restriction fragment length polymorphism (RFLP) pattern or PCR-fingerprint for said first tree by subjecting said nucleic acid of step a) to at least one restriction enzyme and/or standard PCR conditions with at least one specific primer; (c) correlating said RFLP pattern or PCR-fingerprint of step (b) to at least one selected biological and/or biochemical phenotype of said first tree genus and/or species, wherein said phenotype is associated with a genetic locus identified by and/or associated with said RFLP pattern or PCR fingerprint; and (d) identifying orthologous functional gene by sequence homology in the second tree genus and/or species.

In accordance with the present invention there is provided a method of identifying a genetic marker in tree of a second genus and/or species associated with a genetic locus conferring at least one enhanced property selected from the group consisting of fiber length, fiber coarseness, DBH (diameter at breast height), microfibril angle, density, pulp strength, pulp yield, lignin content, pitch propensity, air resistance, kraft pulping H-factor, specific refining energy, wood extractive compounds content and calcium accumulation, said second tree genus and/or species being of different genus and/or species than a first tree species, which comprises the steps of: (a) obtaining a sexually mature parent tree of said first genus and/or species and/or hybrid thereof exhibiting enhanced properties; (b) obtaining a plurality of progeny trees of said parent tree by performing self or cross-pollination; (c) assessing multiple progeny trees for each of a plurality of genetic markers; (d) identifying a genetic marker segregating in an essentially Mendelian ratio and showing linkage with at least some other of said plurality of genetic markers; (e) measuring at least one of said properties in multiple progeny trees; and (f) correlating the presence of enhanced property with a least one marker identified in step (d) as segregating in an essentially Mendelian ratio and showing linkage with at least some of said other markers, the correlation of the presence of enhanced properties with a marker indicating that said marker is associated with a genetic locus conferring enhanced property.

In accordance with the present invention there is provided a method of using a genetic marker for producing a plurality of clonal trees that have at least one enhanced property selected from the group consisting of fiber length, fiber coarseness, DBH (diameter at breast height), microfibril angle, density, pulp strength, pulp yield, lignin content, pitch propensity air resistance, kraft pulping H-factor, specific refining energy, wood extractive compounds content and calcium accumulation, which comprises the steps of: (a) obtaining a sexually mature first parent tree exhibiting enhanced property; (b) obtaining a plurality of progeny trees of said parent tree by performing self or cross-pollination; (c) assessing multiple progeny trees for each of a plurality of genetic markers identified as associated with a genetic locus conferring at least one enhanced property in a second tree of a different genus and/or species than said first parent tree; (d) identifying those genetic markers segregating in an essentially Mendelian ratio in multiple progeny trees; (e) correlating the presence of enhanced property in multiple progeny trees with a least one marker identified in step (d); (f) selecting a progeny tree containing a marker identified in step (d) as associated with a genetic locus conferring enhanced property; and (g) vegetatively propagating said progeny tree selected in step (f) to produce a plurality of clonal trees.

In accordance with the present invention there is provided a stand of clonal enhanced property trees produced in accordance with one aspect of the present invention, the genome of said trees containing the same genetic marker associated with a genetic locus conferring at least one enhanced property in said second tree of a different genus and/or species than said first parent tree.

In accordance with the present invention there is provided a method of using a genetic marker for producing a family of trees wherein at least about half exhibit at least of enhanced property selected from the group consisting of fiber length, fiber coarseness, DBH (diameter at breast height), microfibril angle, density, pulp strength, pulp yield, lignin content, pitch propensity, air resistance, kraft pulping H-factor, specific refining energy, wood extractive compounds content and calcium accumulation, which comprises the steps of: (a) obtaining a sexually mature first parent tree exhibiting enhanced property; (b) obtaining a plurality of progeny trees of said parent tree by performing self or cross-pollination; (c) assessing multiple progeny trees for each of a plurality of genetic markers identified as associated with a genetic locus conferring at least one enhanced property in a second tree of a different genus and/or species than said first parent tree; (d) identifying those genetic markers segregating in an essentially Mendelian ratio in multiple progeny trees; (e) correlating the presence of enhanced property in multiple progeny trees with a least one marker identified in step d); (f) selecting a progeny tree containing a marker identified in step (d) as associated with a genetic locus conferring enhanced property; and (g) sexually propagating said progeny tree selected in step (f) to produce a family of trees, at least about half of said family of trees containing a genetic locus conferring enhanced property and said family of trees exhibiting enhanced property.

In accordance with the present invention there is provided a stand of vegetatively produced enhanced property trees produced in accordance with one aspect of the present invention, the genome of said trees containing the same genetic marker associated with a genetic locus conferring at least one enhanced property in said second tree of a different genus and/or species than said first parent tree.

In accordance with the present invention there is provided a genetic map of QTLs of trees associated with enhanced properties produced in accordance with one aspect of the present invention.

For the purpose of the present invention the following terms are defined below.

The term “Quantitative Trait locus (QTL)” is intended to mean the position(s) occupied on the chromosome by the gene(s) representing a particular trait. The various alternate forms of the gene—that is the alleles used in mapping—all reside at the same location.

The term “restriction fragment linked polymorphism (RFLP)” as used herein means a digestive enzymatic method for detecting localized differences in DNA sequence.

The term “random amplified polymorphic DNA (RAPPED)” as used herein means a PCR based method for detecting localized differences in DNA sequence.

The term “polymerase chain reaction (PCR)” as used herein means a cyclical enzyme-mediated method for making large numbers of identical copies of a stretch of DNA using specific primers.

The term “hybrid thereof” as used herein means a progeny issued from the interbreeding of trees of different breeds, varieties or species especially as produced through tree-breeding for specific genetic and phenotypic characteristics. A hybrid thereof is derived by cross-breeding two different tree species.

The term “candidate gene” as used herein means a sequence of DNA representing a potential gene (an open reading frame, ORF) located within a QTL whose predicted functionality may partially or totally be causal to the given phenotypic trait associated with the QTL.

### BRIEF DESCRIPTION OF THE DRAWINGS

FIG. 1 illustrates SilviScan-2 analysis of hybrid poplar core 331-1062. Data indicate the expected increase in MFA from bark (mature wood zone) to pith (juvenile wood zone). Three scans were performed at resolutions of 1 mm, 2 mm and 5 mm.

FIG. 2 illustrates (a) GC spectrum for acetone extractives from Populus tremuloides (quaking aspen); and (b) GC spectrum for hybrid poplar 331-1016 (F2 TDxTD cross).

FIG. 3 illustrates that accept chips % vs. wood density for selected clones indicates no correlation.

FIG. 4 illustrates bulk density vs. chip density for hybrid poplar chips showing the expected strong correlation.

FIG. 5 illustrates kappa number vs. H-factor: clone 331-1136 which proved difficult to pulp is clearly distinct from the others. Population parents 93-968 and 14-129 form the boundaries of the variability seen in kappa number at each H-factor value.

FIG. 6 illustrates pulp yield vs. kappa number. Parent 93-968 (pure P. trichocarpa) forms a distinct envelope whereas the remainder of the clones examined resemble parent 14-129 (P. deltoides).

FIG. 7 illustrates yield at kappa 17 vs. H-factor to kappa 17.

FIG. 8 illustrates chip density vs. H-factor to kappa 17.

FIG. 9 illustrates fibre coarseness vs. fibre length. The positive correlation seen here is in contrast to that seen for aspen clones but supports the data obtained for the 7^{th} year growth ring from each hybrid poplar in the previous study.

FIG. 10 illustrates chip density vs. fibre length.

FIG. 11 illustrates tensile index vs. bulk. Negative relationship confirms previous aspen data. Most clones show superior strength properties when compared to average values for Eucalyptus species (tensile index 70 N·m/g)

FIG. 12. illustrates histogram of tensile strength and bulk properties for the examined genotypes.

FIG. 13 illustrates tensile index development by PFI beating.

FIG. 14 illustrates tensile index vs. Canadian standard freeness.

FIG. 15 illustrates air resistance (Gurley) vs. sheet density.

FIG. 16 illustrates sheet density vs. Sheffield smoothness.

FIG. 17 illustrates scattering coefficient vs. Canadian standard freeness showing very poor correlation.

FIG. 18 illustrates handsheet deformations caused by calcium deposition.

FIG. 19 illustrates EDS characterization of vessel element mineral deposits.

FIG. 20 illustrates an electron micrograph of vessel element mineral deposition.

FIG. 21 illustrates unscreened Canadian standard freeness vs. specific refining energy and exhibits low, medium and high refining energy demand envelopes at a given freeness value.

FIG. 22 illustrates uptake of NaOH and H₂O₂ vs. specific refining energy and shows that high chemical uptake reduces energy demand at a given freeness of 200 mL.

FIG. 23 illustrates mean chemical uptake vs. chip density and shows that wood density does not affect chemical uptake by hybrid poplar chips.

FIG. 24 illustrates mean chemical uptake vs. tensile index at 200 mL CSF, which indicates that tensile index is dependent upon good chemical impregnation of the chips.

FIG. 25 illustrates uptake vs. wood chip density.

FIG. 26 illustrates fines content vs. scattering coefficient, which indicates high levels of intraclonal variability.

FIG. 27 illustrates mean chemical uptake vs. scattering coefficient and shows that scattering coefficient is negatively dependent on chip chemical consumption.

FIG. 28 illustrates roughness vs. freeness.

FIG. 29 illustrates Sheffield roughness vs. tensile index and shows the wide range of tensile strengths possible at a given roughness value.

FIG. 30 illustrates a genetic map of the hybrid poplar population produced using Mapmaker 3.0 and Mapmaker/QTL 1.1. The 19 Populus linkage groups and positioned RFLP, RAPD and STS markers are shown. Positions of detected QTL which exceed the significance threshold LOD score are indicated by colour-coded vertical bars adjacent to the linkage groups. Phenotyping data colour codes are described in the legend.

### DETAILED DESCRIPTION OF THE INVENTION

Materials and Methods

Sample Sites

Sampling was conducted at the Washington State University Farm 5 plantation site in Puyallup, Washington and at two commercial plantation sites in Northern Oregon at Clatskanie and Boardman. The pedigree sampled was founded in 1981 by interspecific hybridization between Populus trichocarpa (clone 93-968) and P. deltoides (clone ILL-129). Two siblings from the first hybrid generation (F1 family 53), 53-246 and 53-242, were crossed in 1988 to give rise to a family of second generation hybrids used for genetic mapping studies (F2 family 331). Unrooted cuttings of the P, F1 and 55 F2 clones were planted at the sites in a modified randomized complete block design at a 2×2 m spacing. At the time of sampling, the trees were seven (Puyallup) and five (Clatskanie, Boardman) years old.

Tree Sampling

Ten millimetre diameter increment cores were obtained at approximately breast height from 350 surviving trees (90 genotypes) within the pedigree. All cores were removed through the pith from bark to bark. For pilot Kraft pulping analyses, 25 stems were selected—based on the fibre properties and wood density phenotypic data—and harvested from the Puyallup site. The entire stem to a 1″ top size was recovered in each case. Genotyping experiments were performed on DNA extracted from 30 g of live tissue (leaf samples) obtained from each of the 90 sampled genotypes spanning the three growth sites.

Fibre Coarseness and Macerated Pulp Yield

Fibres for analysis were obtained from hand-chipped 10 mm increment cores using an acetic acid/hydrogen peroxide maceration technique (Burkart, L. F. “New technique for maceration of woody tissue,” For. Prod. J. 16, 52 (1966)) whereby a known oven-dried (o.d.) weight of chips was first placed in a test tube, saturated with water then covered in maceration solution (1:1 mixture of glacial acetic acid:hydrogen peroxide (30% from stock bottle)). These samples were then incubated in a dry heating block for 48 hrs. at 60° C. The maceration solution was washed from the chips extensively using distilled water and the pulps disintegrated in a small Hamilton Beach mixer. A dilution series was then used to obtain representative samples of 10,000-20,000 fibres (corresponding to approximately 5 mg of macerated pulp) which were analyzed for length and coarseness values using a Kajaani FS-200 instrument and/or an OpTest Fibre Quality Analyzer. Maceration yields were calculated from oven-dried recovered pulps after fibre analysis.

Microfibril Angle

Microfibril angle (MFA) was measured on 45 whole increment core samples from the family 331 hybrid poplars. The cores were selected on the basis of sufficient size (>20 mm) and soundness of the wood. Prior to analysis, the cores were extracted in denatured ethanol for three days and dried. MFA was determined by SilviScan-2 analysis using scanning X-ray diffractometry (Evans, R. A variance approach to the X-ray diffractometric estimation of microfibril angle in wood. Appita J. 52(4), 283-289 (1999)). Acquisition time was set for 30 seconds to optimize signal to noise ratio and a single diffraction pattern was obtained for each sample to ensure that the entire length of the sample was represented. MFA was estimated from the standard deviation (S) of the 002 azimuthal diffraction profile where: MFA=1.28(S²−36)^{1/2} and S and MFA are both measured in degrees.

Chemical Analyses—Lignin, Extractives (GC/MS) Lignin

Lignin contents were determined for 90 genotypes sampled at the Puyallup growth site. The determinations were performed at the Paprican Pointe Claire facility according to TAPPI standard methods (T13 wd 74).

Extractives Preparation

(Fernandez, M. F., Breuil, C., and Watson, P. A. “A gas-chromatography—mass spectrometry method for the simultaneous determination of wood extractive compounds,” Paprican Pulp and Paper Report 1487. (2000), accepted for publication in J. Chrom.)

The samples were ground in a Wiley Mill at 40 mesh and a 5-6 g o.d. aliquot of the ground wood was placed in a soxhlet thimble and continuously extracted with acetone for 6 hours. The resulting filtrate was concentrated by rotary evaporation and filtered through a pasteur pipette with glass wool, in order to remove any large particulates. The filtrate was then freeze dried, accurately weighed and the resulting crystals re-suspended in acetone to give a concentration of 5,000 ppm based on the total extractives yield. The internal standards, cholesterol palmitate and heptadeptanoic acid (C-17), were added to every one of the extracted samples, at a concentration of 200 ppm. The samples were then transferred to GC vials for analysis of fatty acids by GCMS, using a 10 m DB-XLB column (J&W). The set temperature program started out at 50° C. for 3 minutes, before ramping the temperature up to 340° C. at a rate of 10° C. per minute. This was then followed by maintaining the temperature at 340° C. for 30 minutes and again ramping up to 360° C. at a rate of 10° C. per minute. The injector temperature was held at 320° C. and a constant flow rate of 1.6 mL/minute was maintained. A solvent delay of 5 minutes was set up and data acquisition began at that point. In order for ion detection to occur, a compound table of known retention times was built. Peaks were detected by quant ions (RIC) and integrated. Area ratios were determined relative to the internal standard, cholesterol palmitate.

The peaks were identified and integrated via the compound table that was constructed as a part of the MS data calculations (Fernandez, M P, Watson, P A, Breuil, C. Gas chromatography-mass spectrometry method for the simultaneous determination of wood extractive compounds in quaking aspen. Journal of Chromatography A, 922(ER1-2): 225-233 (2001)).

The resulting area integrations from each spectrum were divided into the internal standard, cholesterol palmitate, to give a ratio. This relative number was then used on a peak specific basis (peak identification by retention time) as phenotypic data for genetic mapping experiments. The area of particular interest falls between 25 to 40 minutes and contains the waxes, sterols and steryl esters, the major components of pitch in wood.

Pulps Preparation

Wood Chip Preparation

Selected wood logs from the 25 hybrid poplar clones from the base up to a 1″ top diameter were debarked, slabbed (if necessary to reduce the diameter) on a portable Woodmizer LT-15 sawmill and chipped using a 36″ CM&E 10-knife industrial disc chipper. A portion of the chips were air-dried and later screened in a Wennberg chip classifier to obtain chips in the thickness range of 2-6 mm for chemical pulping. These accept chips were used in the kraft cooks. The remaining green chips were screened on a BM&H vibratory screen to remove over sized chips and fines prior to mechanical pulping.

Kraft Pulping

Three representative aliquots of air-dried accept chips from each of the samples were kraft pulped in bombs [45 g oven-dried (o.d.) charge] within a B-K micro-digester assembly. The cooking conditions were as follows:

| Time to maximum temperature | 135 min |
|---|---|
| Maximum cooking temperature | 170° C. |
| Effective alkali, % OD weight of wood | 13% |
| % Sulphidity | 25% |
| Liquor to wood ratio | 5:1 |
| H factor | 700-1400 |

All of the pulps produced were washed, oven-dried and weighed to determine pulp yield. Kappa number and black liquor residual effective alkali were determined by TAPPI standard procedures (T236 cm 85 and T625 respectively). From these results the optimum cooking conditions required to produce pulps at 17 Kappa number were estimated by fitting regression lines through each set of data (r²≧0.95). Large quantities of kraft pulp were subsequently produced in a 28 L Weverk laboratory digester. The pulps produced were disintegrated, washed and screened through an 8-cut screen plate.

A PFI mill was used to prepare 5-point beating curves for each pulp sample by refining at: 0, 1000, 3000, 6000 revolutions (CPPA Standard C.7). A disintegrator (CPPA Standard C.9P) and a stainless steel sheet machine were used for testing and forming all sets of handsheets (CPPA Standard C.4 and C.5). All physical and optical testing was performed in a constant temperature and humidity room, using CPPA standard methods.

Alkaline Peroxide Refiner Mechanical Pulping (APRMP)

Two-stage impregnation of twenty-four hybrid poplar chips samples was carried out using a Sunds Defibrator Prex impregnator with a 3:1 compression ratio.

Stage One

Chips were steamed at atmospheric pressure for 10 min to expel entrapped air from the chips and replace it with water vapour. Impregnation with a solution containing 0.25% DTPA (diethylenetriamine pentaacetic acid) was carried out in the Prex impregnator. This provided a chemical charge of 0.26% to 0.66% DTPA on o.d. wood.

Stage Two

First-stage impregnated chips were further impregnated with a solution containing 0.25% MgSO₄, 2.0% Na₂SiO₃, 2.35% NaOH and 1.5% H₂O₂. This resulted in chemical charges as follows:
MgSO₄ applied, % o.d. wood: 0.36 to 0.69
Na₂SiO₃ applied, % o.d. wood: 2.29 to 5.45
NaOH applied, % o.d. wood: 3.69 to 5.89
H₂O₂ applied, % o.d. wood: 1.72 to 3.76

After 60 min retention at 60° C. the side port of the preheater was opened to remove the impregnated chips for open-discharge refining in a 30.5 cm single-disc Sprout Waldron laboratory refiner to prepare alkaline peroxide refiner mechanical pulps (APRMP). Each chip sample was refined at three energy levels to give 72 APRMP pulps in the freeness range from 144 to 402 mL Csf. Immediately after first pass open-discharge refining the pulp stock was neutralized to pH 4.5-4.8. Wood chip density and chemical uptake of hybrid poplar chip samples are shown in Table I.

TABLE I
Chip density and chemical uptake for APRMP pulps

| | Chip Density^{a} | NaOH | H₂O₂ |
|---|---|---|---|
| Sample No. | (kg/m³) | (% o.d. wood) | (% o.d. wood) |
| 14-129 (1) | 285 | 5.39 | 3.44 |
| 14-129 (2) | 304 | 6.07 | 3.88 |
| 53-242 (1) | 329 | 5.13 | 3.27 |
| 53-242 (2) | 302 | 4.41 | 2.82 |
| 53-246 (1) | 311 | 6.24 | 3.99 |
| 54-246 (2) | 325 | 4.57 | 2.92 |
| 93-968 (1) | 303 | 4.20 | 2.68 |
| 93-968 (2) | 314 | 3.80 | 2.43 |
| 331-1059 (2) | 303 | 4.63 | 2.95 |
| 331-1059 (3) | 302 | 4.59 | 2.93 |
| 331-1061 (1) | 338 | 6.40 | 4.09 |
| 331-1061 (2) | 328 | 5.41 | 3.46 |
| 331-1061 (3) | 345 | 4.35 | 2.78 |
| 331-1062 (1) | 280 | 4.20 | 2.68 |
| 331-1062 (2) | 290 | 6.51 | 4.24 |
| 331-1075 (2) | 300 | 3.39 | 2.16 |
| 331-1093 (1) | 279 | 4.23 | 2.70 |
| 331-1093 (2) | 288 | 5.38 | 3.43 |
| 331-1118 (1) | 346 | 5.89 | 3.76 |
| 331-1118 (2) | 373 | 3.42 | 2.18 |
| 331-1122 (1) | 283 | 3.80 | 2.43 |
| 331-1126 (1) | 386 | 2.69 | 1.72 |
| 331-1162 (3) | 336 | 4.22 | 2.69 |
| 331-1186 (3) | 292 | 4.69 | 3.00 |

Chip thickness = 2-6 mm

Other pertinent refining conditions are shown below.

| | |
|---|---|
| Number of passes: | 2 to 4 depending upon freeness |
| | level |
| Nominal plate gap: | 0.38 mm (first pass); 0.03 to |
| | 0.2 mm (subsequent pass) |
| Refining consistency: | 18 to 23% o.d. pulp |

After latency removal, each pulp was screened on a 6-cut laboratory flat screen to determine screen rejects. Bauer-McNett fibre classifications on screened pulps were determined. Representative samples from each of the 72 pulp samples were analyzed for fibre length using a Kajaani FS-200 instrument. Handsheets were prepared with white water recirculation to minimize the loss of fines and tested for bulk, mechanical, and optical properties using CPPA standard methods. Handsheet roughness was measured in Sheffield units (SU).

Assessment of Calcium Accumulation

The nature of the observed kraft pulp handsheet deformations was explored by both light and electron microscopy and by energy-dispersive X-ray analysis. Wood chip deposits were characterized in similar fashion. The methodologies used have been described in a previous report (Potter, S. (2000) “Calcium accumulation in the wood of short-rotation cottonwood species: Effects on pulp properties”, 21^{st} Session of the International Poplar Commission (IPC-2000): poplar and willow culture: meeting the needs of society and the environment, Sep. 24-28, 2000, Vancouver, Wash.

Genetic Map Construction and QTL Mapping

The Populus genetic map used in this study, previously constructed using the same family 331 pedigree, consists of 342 RFLP, STS and RAPD markers and is described in (Bradshaw, H. D., Villar, M., Watson, B. D., Otto, K. G., and Stewart, S. “Molecular genetics of growth and development in Populus III. A genetic linkage map of a hybrid poplar composed of RFLP, STS and RAPD markers,” Theor. Appl. Genet. 89, 551-558 (1994)). The 19 large linkage groups, corresponding closely to the 19 Populus chromosomes, were scanned for the phenotypic data obtained using the program MAPMAKER-QTL 1.1 (Patterson, A. H., Lander, E. S., Hewitt, J. D., Peterson, S., and Lincoln, S. E. “Resolution of quantitative traits into Mendelian factors by using a complete linkage map of restriction fragment linked polymorphisms,” Nature 335, 721-726 (1988)). Based on the scanned genome length and the distance between genetic markers, a logarithmic odds (LOD) significance threshold level of 2.9 was chosen (this ensures that the chance of a false positive QTL being detected is at most 5%). For more details on the QTL mapping procedure employed see (Bradshaw, H. D. and Stettler, R. F. “Molecular genetics of growth and development in Populus IV. Mapping QTLs with large effects on growth, form, and phenology traits in a forest tree,” Genetics 139, 963-973 (1995)).

RAPD Analysis, Polymerase Chain Reaction (PCR) and Product Cloning

For each trait examined, QTL-associated markers were identified from the genetic map and were employed to generate polymorphic products from phenotypically selected F₂ generation individuals. Random Amplified Polymorphic DNA (RAPD) markers were purchased from Operon Technologies Inc. (Alameda, Calif., U.S.A.) and Restriction Fragment Linked Polymorphism (RFLP) markers were constructed from published sequence data by the Biotechnology Laboratory at the University of British Columbia.

Both types of markers were used in standard PCR reactions to generate polymorphic amplified product bands corresponding to the QTL-linked markers identified on the genetic map. PCR conditions were standard for RAPD analyses (H. D. Bradshaw, personal communication) and performed using rTaq polymerase (Amersham-Pharmacia) and a Techne Genius thermal cycler. Cycle conditions were as follows:

| | | |
|---|---|---|
| STEP | 1: | 94° C., 3 min |
| | 2: | 94° C., 5 sec |
| | 3: | 36° C., 30 sec |
| | 4: | 72° C., 1 min |
| | 5: | Repeat 2-4, 34× |
| | 6: | 4° C., hold. |

PCR products from the phenotypically selected F2 generation individuals were separated on 1% agarose gels according to standard methods (Sambrook, J., Fritsch, E. F., and Maniatis, T. Molecular cloning. A laboratory manual, 2^{nd} Ed., Cold Spring Harbor Laboratory Press (1990)) and polymorphic bands of the appropriate size were excised from the gels. Products were purified from the agarose using the Amersham-Pharmacia GFX PCR gel band purification kit and cloned into the Promega pGEM-T vector system (with supplied competent cells) according to manufacturers' protocols and standard blue/white selection cloning procedures on ampicillin agar. Cloned PCR products were prepared from transformed cells using the Promega Wizard Plus miniprep kit, again according to the manufacturers protocols, and were then sequenced at the Biotechnology Laboratory, University of British Columbia.

Results and Discussion

Fibre Coarseness, Microfibril Angle and Macerated Pulp Yield

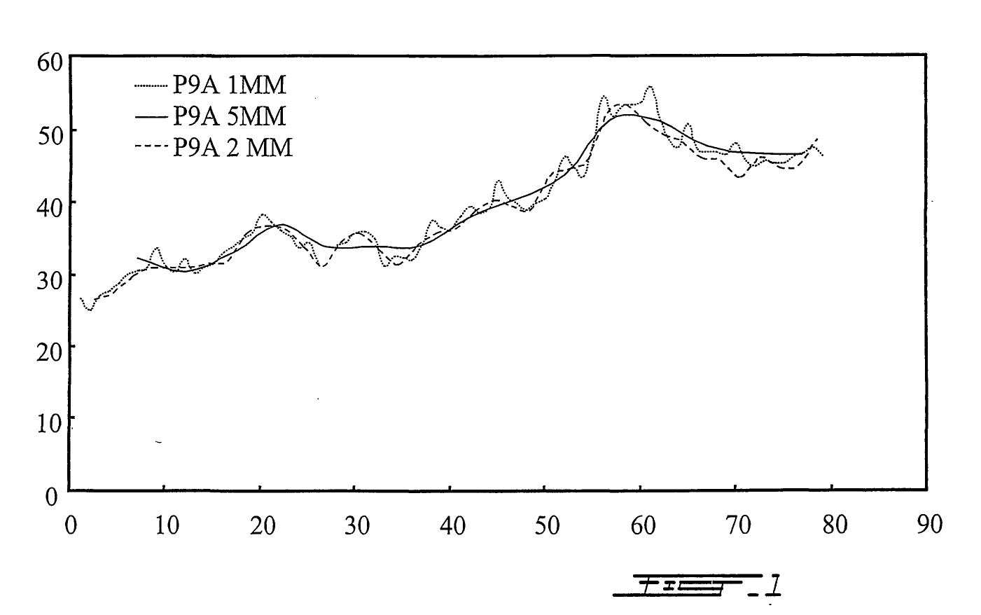

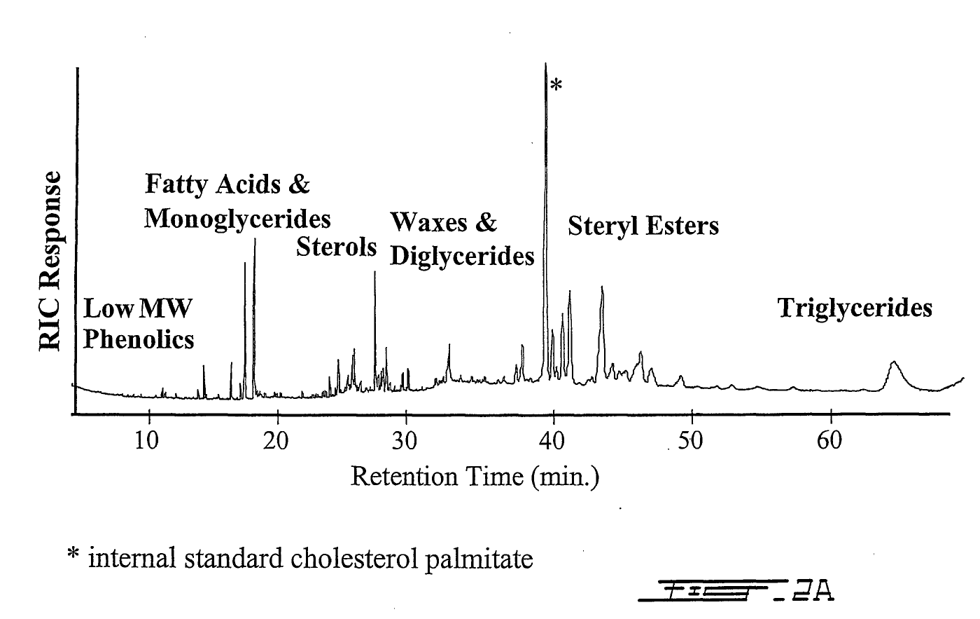

Fibre length and coarseness and macerated pulp yield data were obtained on core samples for each of the 350 trees sampled in the study using the pulp maceration technique and either the Kajaani FS-200 or the automated OpTest FQA instruments and are presented in Table II. Previous experiments have shown no difference in the fibre properties analyses of poplar samples between these two instruments (Robertson, G., Olson, J., Allen, P., Chan, B. and Seth, R. “Measurement of fibre length, coarseness and shape with the fibre quality analyzer”. TAPPI J. 82(10), 93-98 (1999)). The outermost ring (age 7) data are presented in Table II. Microfibril angle data for the outermost ring of each core (i.e. age 7), obtained using the SilviScan-2 technique, are also presented in Table III. FIG. 1 shows the results of a typical SilviScan-2 analysis of an increment core sample from bark to pith at different levels of scanning resolution.

TABLE II
Fibre length, coarseness and macerated
pulp yield data for trees sampled

| Clone ID | Yield | Fibre Length | Coarseness | Site |
|---|---|---|---|---|
| 14-129 | 46.2 | 0.84 | 0.065 | Puyallup |
| 14-129 | 44.1 | 0.76 | 0.085 | |
| 93-968 | 50.8 | 0.99 | 0.095 | |
| 93-968 | 49.9 | 0.97 | 0.112 | |
| 93-968 | 50.5 | 0.98 | 0.102 | |
| 53-242 | 50.9 | 0.85 | 0.082 | |
| 53-242 | 47 | 0.83 | 0.069 | |
| 53-242 | 52.4 | 0.79 | 0.076 | |
| 53-246 | 50.9 | 0.83 | 0.065 | |
| 53-246 | 46.8 | 0.84 | 0.082 | |
| 331-1059 | 55.9 | 0.83 | 0.083 | |
| 331-1059 | 47.6 | 0.74 | 0.075 | |
| 331-1059 | 56.1 | 0.8 | 0.079 | |
| 331-1061 | 51.8 | 0.96 | 0.095 | |
| 331-1061 | 43.4 | 0.89 | 0.086 | |
| 331-1061 | 50.7 | 0.93 | 0.098 | |
| 331-1062 | 49.3 | 1.01 | 0.102 | |
| 331-1062 | 45.5 | 1 | 0.118 | |
| 331-1062 | 39.7 | 0.97 | 0.095 | |
| 331-1065 | 45.8 | 0.78 | 0.088 | |
| 331-1065 | 50.8 | 0.82 | 0.076 | |
| 331-1065 | 55.8 | 0.81 | 0.055 | |
| 331-1060 | 47.8 | 0.85 | 0.1037 | |
| 331-1060 | 51.8 | 0.81 | 0.089 | |
| 331-1064 | 55.9 | 0.98 | 0.064 | |
| 331-1064 | 48.9 | 0.96 | 0.083 | |
| 331-1067 | 52.3 | 0.82 | 0.064 | |
| 331-1067 | 47.6 | 0.87 | 0.063 | |
| 331-1067 | 53.2 | 0.87 | 0.066 | |
| 331-1069 | 49.6 | 0.89 | 0.095 | |
| 331-1069 | 56.1 | 0.99 | 0.1131 | |
| 331-1072 | 49.6 | 0.84 | 0.054 | |
| 331-1073 | 54.4 | 0.69 | 0.061 | |
| 331-1075 | 51.8 | 0.91 | 0.092 | |
| 331-1075 | 51 | 0.88 | 0.097 | |
| 331-1075 | 54.7 | 0.91 | 0.085 | |
| 331-1076 | 43.4 | 0.74 | 0.068 | |
| 331-1076 | 53.6 | 0.76 | 0.085 | |
| 331-1077 | 54.2 | 0.77 | 0.038 | |
| 331-1078 | 50.7 | 0.87 | 0.085 | |
| 331-1078 | 51 | 0.85 | 0.08 | |
| 331-1079 | 55.6 | 0.98 | 0.085 | |
| 331-1079 | 49.3 | 0.89 | 0.1 | |
| 331-1079 | 47.6 | 0.95 | 0.092 | |
| 331-1084 | 51.3 | 0.91 | 0.085 | |
| 331-1084 | 45.5 | 0.85 | 0.074 | |
| 331-1086 | 44.1 | 0.82 | 0.083 | |
| 331-1086 | 48.9 | 0.87 | 0.079 | |
| 331-1087 | 39.7 | 0.86 | 0.079 | |
| 331-1087 | 39.3 | 0.85 | 0.066 | |
| 331-1087 | 54.6 | 0.84 | 0.085 | |
| 331-1090 | 45 | 0.95 | 0.076 | |
| 331-1093 | 44.5 | 0.91 | 0.085 | |
| 331-1093 | 48.5 | 0.75 | 0.085 | |
| 331-1093 | 45.8 | 0.81 | 0.065 | |
| 331-1095 | 49.1 | 0.91 | 0.068 | |
| 331-1095 | 50.8 | 0.77 | 0.091 | |
| 331-1101 | 47.2 | 0.93 | 0.095 | |
| 331-1101 | 55.2 | 0.96 | 0.082 | |
| 331-1101 | 55.8 | 0.92 | 0.076 | |
| 331-1102 | 43.5 | 0.73 | 0.073 | |
| 331-1102 | 47.7 | 0.82 | 0.083 | |
| 331-1103 | 46.2 | 0.81 | 0.085 | |
| 331-1103 | 49.6 | 0.92 | 0.09 | |
| 331-1103 | 50.7 | 0.95 | 0.081 | |
| 331-1104 | 44.1 | 0.81 | 0.066 | |
| 331-1104 | 51.5 | 0.82 | 0.054 | |
| 331-1106 | 52 | 0.68 | 0.075 | |
| 331-1106 | 50.8 | 0.79 | 0.068 | |
| 331-1112 | 48.2 | 0.6 | 0.077 | |
| 331-1112 | 50.8 | 0.75 | 0.078 | |
| 331-1112 | 49.9 | 0.76 | 0.065 | |
| 331-1114 | 51.3 | 0.87 | 0.102 | |
| 331-1114 | 48.1 | 0.98 | 0.099 | |
| 331-1114 | 50.5 | 0.99 | 0.118 | |
| 331-1118 | 46.9 | 0.81 | 0.098 | |
| 331-1118 | 51 | 0.99 | 0.078 | |
| 331-1120 | 50.9 | 0.83 | 0.065 | |
| 331-1121 | 48.2 | 0.54 | 0.055 | |
| 331-1122 | 51.9 | 0.84 | 0.062 | |
| 331-1122 | 47 | 0.78 | 0.077 | |
| 331-1122 | 45.9 | 0.77 | 0.064 | |
| 331-1126 | 52.7 | 1.03 | 0.113 | |
| 331-1126 | 52.4 | 0.98 | 0.099 | |
| 331-1126 | 44 | 0.93 | 0.098 | |
| 331-1127 | 47.2 | 0.87 | 0.102 | |
| 331-1127 | 50.9 | 1.01 | 0.124 | |
| 331-1127 | 48.4 | 1.02 | 0.075 | |
| 331-1128 | 53.2 | 0.9 | 0.085 | |
| 331-1128 | 46.8 | 0.85 | 0.085 | |
| 331-1128 | 39.7 | 0.85 | 0.082 | |
| 331-1130 | 47.8 | 0.85 | 0.083 | |
| 331-1130 | 48.9 | 0.92 | 0.079 | |
| 331-1130 | 51.8 | 0.93 | 0.103 | |
| 331-1131 | 44.1 | 0.99 | 0.098 | |
| 331-1131 | 55.9 | 0.96 | 0.085 | |
| 331-1133 | 45.5 | 0.76 | 0.078 | |
| 331-1133 | 48.9 | 0.69 | 0.069 | |
| 331-1136 | 51.3 | 0.64 | 0.077 | |
| 331-1136 | 52.3 | 0.69 | 0.082 | |
| 331-1140 | 56.1 | 0.8 | 0.081 | |
| 331-1140 | 54.2 | 0.78 | 0.086 | |
| 331-1149 | 51 | 0.91 | 0.123 | |
| 331-1149 | 46.9 | 0.94 | 0.122 | |
| 331-1149 | 55.2 | 0.94 | 0.118 | |
| 331-1151 | 45.8 | 0.77 | 0.042 | |
| 331-1151 | 47.6 | 0.94 | 0.106 | |
| 331-1151 | 54.4 | 0.91 | 0.117 | |
| 331-1158 | 48.1 | 0.75 | 0.064 | |
| 331-1158 | 51 | 0.7 | 0.083 | |
| 331-1158 | 52 | 0.77 | 0.075 | |
| 331-1162 | 50.7 | 0.81 | 0.078 | |
| 331-1162 | 47.2 | 0.89 | 0.087 | |
| 331-1162 | 52.4 | 0.94 | 0.085 | |
| 331-1163 | 50.5 | 0.62 | 0.05 | |
| 331-1163 | 48.3 | 0.65 | 0.054 | |
| 331-1169 | 44.8 | 0.71 | 0.085 | |
| 331-1169 | 47.9 | 0.78 | 0.091 | |
| 331-1169 | 45.9 | 0.8 | 0.121 | |
| 331-1173 | 49.9 | 0.96 | 0.092 | |
| 331-1173 | 49.1 | 0.81 | 0.075 | |
| 331-1173 | 50.8 | 0.91 | 0.092 | |
| 331-1174 | 46.2 | 0.85 | 0.093 | |
| 331-1174 | 51.2 | 0.88 | 0.124 | |
| 331-1182 | 47.6 | 0.88 | 0.091 | |
| 331-1186 | 45.8 | 0.91 | 0.089 | |
| 331-1186 | 52.6 | 0.95 | 0.084 | |
| 331-1186 | 44.9 | 0.99 | 0.077 | |
| 331-1580 | 53.2 | 0.67 | 0.075 | |
| 331-1580 | 51.7 | 0.72 | 0.081 | |
| 331-1580 | 48.1 | 0.79 | 0.066 | |
| 331-1582 | 46.8 | 0.86 | 0.075 | |
| 331-1582 | 51.6 | 1.01 | 0.068 | |
| 331-1582 | 47.3 | 0.94 | 0.075 | |
| 331-1587 | 52.8 | 0.85 | 0.076 | |
| 331-1587 | 50.5 | 0.91 | 0.075 | |
| 14-1292.1 | 34.8 | 0.77 | 0.077 | Boardman - B |
| 14-1293.2 | 39.6 | 0.75 | 0.095 | Clatskanie - C |
| 14-1294.1B | 42.3 | 0.67 | 0.113 | |
| 93-9682.2 | 46.9 | 0.86 | 0.11 | |
| 93-9682.1 | 42.3 | 0.79 | 0.092 | |
| 93-9683.1 | 45.9 | 0.72 | 0.105 | |
| 93-9683.2 | 49 | 0.73 | 0.088 | |
| 93-9684.1B | 55.5 | 0.79 | 0.098 | |
| 93-9684.2B | 58.3 | 0.81 | 0.1 | |
| 53-2422.2 | 53.3 | 0.74 | 0.101 | |
| 53-2423.1 | 51 | 0.71 | 0.087 | |
| 53-2423.2 | 53.2 | 0.7 | 0.12 | |
| 53-2424.1B | 53.2 | 0.71 | 0.08 | |
| 53-2461.1 | 46.2 | 0.65 | 0.18 | |
| 53-2461.2 | 46.3 | 0.66 | 0.097 | |
| 53-2462.1 | 50.6 | 0.64 | 0.087 | |
| 53-2464.1B | 56.7 | 0.68 | 0.073 | |
| 10591.1 | 28 | 0.6 | 0.087 | |
| 10591.2 | 37.2 | 0.6 | 0.146 | |
| 10593.2B | 58.1 | 0.61 | 0.103 | |
| 10594.1B | 46.2 | 0.73 | 0.123 | |
| 10601.1 | 47.5 | 0.6 | 0.094 | |
| 10601.2 | 50 | 0.61 | 0.106 | |
| 10602.1 | 49.5 | 0.66 | 0.119 | |
| 10602.2 | 54.2 | 0.72 | 0.111 | |
| 10603.2B | 48.3 | 0.54 | 0.057 | |
| 10604.2B | 43.3 | 0.56 | 0.099 | |
| 10611.1 | 47.9 | 0.79 | 0.079 | |
| 10611.2 | 47.6 | 0.79 | 0.117 | |
| 10614.1B | 47.7 | 0.79 | 0.097 | |
| 10614.2B | 50 | 0.75 | 0.044 | |
| 10622.1 | 49.5 | 0.79 | 0.114 | |
| 10622.2 | 54.2 | 0.71 | 0.097 | |
| 10624.1B | 48.3 | 0.59 | 0.068 | |
| 10624.2B | 43.3 | 0.51 | 0.092 | |
| 10653.1 | 47.9 | 0.69 | 0.105 | |
| 10653.2 | 47.6 | 0.66 | 0.094 | |
| 10654.1B | 47.7 | 0.74 | 0.156 | |
| 10654.2B | 34.8 | 0.74 | 0.175 | |
| 10671.1 | 34.8 | 0.68 | 0.098 | |
| 10671.2 | 39.6 | 0.68 | 0.128 | |
| 10674.1B | 42.3 | 0.64 | 0.075 | |
| 10674.2B | 46.9 | 0.65 | 0.071 | |
| 10693.1 | 42.3 | 0.67 | 0.211 | |
| 10693.1B | 45.9 | 0.69 | 0.139 | |
| 10693.2B | 47.5 | 0.68 | 0.129 | |
| 10721.2 | 50 | 0.63 | 0.138 | |
| 10722.1 | 49.5 | 0.72 | 0.147 | |
| 10722.2 | 54.2 | 0.71 | 0.131 | |
| 10724.1B | 48.3 | 0.72 | 0.139 | |
| 10724.2B | 43.3 | 0.67 | 0.149 | |
| 10731.1 | 47.9 | 0.66 | 0.242 | |
| 10731.2 | 47.6 | 0.65 | 0.241 | |
| 10732.2 | 47.7 | 0.64 | 0.234 | |
| 10734.1B | 38 | 0.67 | 0.201 | |
| 10734.2B | 43.2 | 0.56 | 0.111 | |
| 10751.1 | 27.2 | 0.66 | 0.114 | |
| 10751.2 | 50.6 | 0.74 | 0.076 | |
| 10754.1B | 50 | 0.78 | 0.087 | |
| 10754.2B | 48.7 | 0.66 | 0.108 | |
| 10762.1 | 50.4 | 0.55 | 0.11 | |
| 10762.2 | 41 | 0.63 | 0.131 | |
| 10772.2 | 49.5 | 0.56 | 0.103 | |
| 10781.1 | 43.1 | 0.48 | 0.135 | |
| 10781.2 | 50.4 | 0.52 | 0.219 | |
| 10784.1B | 47.3 | 0.72 | 0.214 | |
| 10784.2B | 36.7 | 0.6 | 0.186 | |
| 10791.1 | 53 | 0.69 | 0.114 | |
| 10791.2 | 58.1 | 0.7 | 0.123 | |
| 10794.1B | 46.2 | 0.75 | 0.107 | |
| 10794.2B | 47.5 | 0.82 | 0.114 | |
| 10841.1 | 50 | 0.74 | 0.093 | |
| 10841.2 | 49.5 | 0.73 | 0.108 | |
| 10844.1B | 54.2 | 0.69 | 0.081 | |
| 10844.2B | 48.3 | 0.74 | 0.094 | |
| 10861.1 | 43.3 | 0.7 | 0.086 | |
| 10861.2 | 47.9 | 0.62 | 0.113 | |
| 10864.1B | 47.6 | 0.62 | 0.114 | |
| 10864.2B | 47.7 | 0.59 | 0.132 | |
| 10872.1 | 34.8 | 0.69 | 0.113 | |
| 10872.2 | 39.6 | 0.71 | 0.083 | |
| 10874.1B | 42.3 | 0.73 | 0.093 | |
| 10874.2B | 46.9 | 0.72 | 0.095 | |
| 10902.1 | 42.3 | 0.79 | 0.089 | |
| 10902.2 | 45.9 | 0.74 | 0.067 | |
| 10904.1B | 55.5 | 0.87 | 0.091 | |
| 10904.2B | 58.3 | 0.8 | 0.083 | |
| 10931.1 | 53.3 | 0.62 | 0.099 | |
| 10931.2 | 51 | 0.58 | 0.075 | |
| 10934.1B | 53.2 | 0.67 | 0.095 | |
| 10934.2B | 53.2 | 0.63 | 0.086 | |
| 10951.1 | 46.2 | 0.63 | 0.093 | |
| 10951.2 | 46.3 | 0.8 | 0.109 | |
| 10954.1B | 50.6 | 0.77 | 0.084 | |
| 10954.2B | 56.7 | 0.62 | 0.082 | |
| 11011.1 | 28 | 0.76 | 0.093 | |
| 11012.2 | 37.2 | 0.73 | 0.104 | |
| 11014.2B | 58.1 | 0.74 | 0.099 | |
| 11021.1 | 46.2 | 0.69 | 0.109 | |
| 11021.2 | 46.2 | 0.68 | 0.081 | |
| 11023.1B | 47.5 | 0.7 | 0.093 | |
| 11031.1 | 50 | 0.74 | 0.084 | |
| 11031.2 | 49.5 | 0.73 | 0.086 | |
| 11034.1B | 54.2 | 0.74 | 0.091 | |
| 11034.2B | 48.3 | 0.85 | 0.09 | |
| 11041.1 | 43.3 | 0.73 | 0.113 | |
| 11041.2 | 47.9 | 0.74 | 0.073 | |
| 11044.1B | 47.6 | 0.72 | 0.101 | |
| 11044.2B | 47.7 | 0.7 | 0.087 | |
| 11121.1 | 38 | 0.5 | 0.12 | |
| 11123.1 | 43.2 | 0.62 | 0.08 | |
| 11124.2B | 27.2 | 0.38 | 0.18 | |
| 11142.1 | 50.6 | 0.74 | 0.097 | |
| 11142.2 | 50 | 0.78 | 0.087 | |
| 11144.1B | 48.7 | 0.76 | 0.073 | |
| 11144.2B | 50.4 | 0.86 | 0.087 | |
| 11181.1 | 41 | 0.6 | 0.146 | |
| 11182.1 | 49.5 | 0.68 | 0.103 | |
| 11184.1B | 43.1 | 0.56 | 0.123 | |
| 11184.2B | 50.4 | 0.6 | 0.094 | |
| 11201.1 | 50.9 | 0.65 | 0.106 | |
| 11201.2 | 47.3 | 0.55 | 0.119 | |
| 11211.1 | 49.3 | 0.59 | 0.111 | |
| 11214.1B | 46.1 | 0.64 | 0.057 | |
| 11214.2B | 45.4 | 0.66 | 0.099 | |
| 11221.1 | 44.1 | 0.63 | 0.079 | |
| 11221.2 | 49.1 | 0.62 | 0.117 | |
| 11223.2B | 44 | 0.59 | 0.097 | |
| 11224.2B | 51.2 | 0.57 | 0.044 | |
| 11261.2 | 44.3 | 0.78 | 0.114 | |
| 11262.1 | 51.3 | 0.82 | 0.097 | |
| 11264.1B | 47.3 | 0.86 | 0.068 | |
| 11264.2B | 36.7 | 0.8 | 0.092 | |
| 11271.1 | 46.1 | 0.69 | 0.105 | |
| 11271.2 | 45.4 | 0.71 | 0.094 | |
| 11274.1B | 46.6 | 0.66 | 0.156 | |
| 11274.2B | 43.2 | 0.62 | 0.175 | |
| 11282.1 | 35 | 0.79 | 0.098 | |
| 11282.2 | 52.4 | 0.78 | 0.128 | |
| 11284.1B | 50 | 0.82 | 0.075 | |
| 11284.2B | 39 | 0.87 | 0.071 | |
| 11302.1 | 39.6 | 0.72 | 0.211 | |
| 11302.2 | 42.3 | 0.66 | 0.139 | |
| 11311.1 | 46.9 | 0.71 | 0.129 | |
| 11311.2 | 42.3 | 0.73 | 0.138 | |
| 11312.1 | 45.9 | 0.64 | 0.147 | |
| 11313.2B | 27.2 | 0.67 | 0.131 | |
| 11334.1B | 50.6 | 0.64 | 0.139 | |
| 11334.2B | 44.3 | 0.65 | 0.149 | |
| 11361.1 | 45.4 | 0.6 | 0.242 | |
| 11361.2 | 44.1 | 0.56 | 0.241 | |
| 11362.2 | 49.1 | 0.53 | 0.234 | |
| 11401.1 | 44 | 0.55 | 0.201 | |
| 11402.1 | 51.2 | 0.61 | 0.111 | |
| 11402.2 | 44.3 | 0.62 | 0.114 | |
| 11404.1B | 51.3 | 0.64 | 0.076 | |
| 11404.2B | 51.4 | 0.74 | 0.087 | |
| 11491.1 | 37.1 | 0.74 | 0.108 | |
| 11491.2 | 49.4 | 0.79 | 0.11 | |
| 11492.1 | 50.8 | 0.68 | 0.131 | |
| 11494.1B | 35.5 | 0.65 | 0.103 | |
| 11494.2B | 46.5 | 0.7 | 0.135 | |
| 11511.1 | 47.2 | 0.59 | 0.219 | |
| 11511.2 | 46.6 | 0.71 | 0.214 | |
| 11511.22 | 43.2 | 0.69 | 0.186 | |
| 11514.1B | 35 | 0.8 | 0.114 | |
| 11581.1 | 52.4 | 0.62 | 0.123 | |
| 11581.2 | 50 | 0.67 | 0.107 | |
| 11583.1B | 39 | 0.61 | 0.114 | |
| 11583.2B | 51.4 | 0.77 | 0.093 | |
| 11584.2B | 37.1 | 0.59 | 0.108 | |
| 11621.2 | 49.4 | 0.7 | 0.081 | |
| 11622.1 | 50.8 | 0.69 | 0.094 | |
| 11624.1B | 35.5 | 0.48 | 0.086 | |
| 11631.1 | 46.5 | 0.61 | 0.113 | |
| 11631.2 | 47.2 | 0.64 | 0.114 | |
| 11634.1B | 46.6 | 0.48 | 0.132 | |
| 11634.2B | 43.2 | 0.54 | 0.113 | |
| 11653.1 | 35 | 0.53 | 0.083 | |
| 11691.1 | 52.4 | 0.63 | 0.093 | |
| 11691.2 | 49.3 | 0.55 | 0.095 | |
| 11694.1B | 58.9 | 0.66 | 0.089 | |
| 11694.2B | 52.2 | 0.69 | 0.067 | |
| 11732.1 | 49.8 | 0.6 | 0.091 | |
| 11732.2 | 46.5 | 0.65 | 0.083 | |
| 11733.1 | 46.6 | 0.56 | 0.099 | |
| 11733.2 | 50.3 | 0.61 | 0.075 | |
| 11734.1B | 47.6 | 0.69 | 0.095 | |
| 11734.2B | 48.4 | 0.67 | 0.086 | |
| 11741.1 | 52.7 | 0.68 | 0.093 | |
| 11741.2 | 48 | 0.57 | 0.109 | |
| 11862.1 | 50.9 | 0.74 | 0.084 | |
| 11862.2 | 47.3 | 0.7 | 0.082 | |
| 15803.1 | 49.3 | 0.6 | 0.093 | |
| 15803.2 | 46.1 | 0.53 | 0.104 | |
| 15804.1B | 45.4 | 0.62 | 0.099 | |
| 15804.2B | 44.1 | 0.65 | 0.109 | |
| 15823.1 | 49.1 | 0.78 | 0.081 | |
| 15823.2 | 44 | 0.74 | 0.093 | |
| 15823.1B | 51.2 | 0.72 | 0.084 | |
| 15823.2B | 44.3 | 0.66 | 0.086 | |
| 15871.1 | 51.3 | 0.69 | 0.091 | |
| 15871.2 | 47.3 | 0.65 | 0.09 | |
| 15874.1B | 36.7 | 0.49 | 0.113 | |
| 15874.2B | 53 | 0.63 | 0.073 | |

Significant variability is seen for the three traits—fibre coarseness ranges from 0.042 mg/m to 0.219 mg/m; microfibril angle from 17.80 to 38.20; maceration yield from 27.2% to 56.1%. Results of the Mapmaker-QTL 1.1 analysis of the data are shown in Table IV. One significant QTL has been found for fibre coarseness, one low significance QTL for microfibril angle and four for macerated pulp yield. The QTL for each fibre property are coincident and one of the QTL for maceration yield (P1027_P192/R) is coincident with the low significance QTL detected for Kraft pulp yield (Table IV). These regions may, therefore, represent particularly important areas of the genome for pulp and paper properties.

TABLE III
Microfibril angle data for hybrid poplars at age 7

| TREE | MFA |
|---|---|
| 331- | Ring 7 data |
| 1060 | 29.22 |
| 1063 | 26.15 |
| 1064 | 30.45 |
| 1065 | 27.13 |
| 1067 | 32.09 |
| 1069 | 29.09 |
| 1072 | 30.06 |
| 1073 | 32.24 |
| 1075 | 33.55 |
| 1076 | 34.60 |
| 1078 | 29.58 |
| 1079 | 31.25 |
| 1080 | 23.40 |
| 1082 | 28.43 |
| 1084 | 28.90 |
| 1095 | 30.58 |
| 1101 | 25.48 |
| 1103 | 28.03 |
| 1104 | 35.26 |
| 1114 | 21.56 |
| 1120 | 25.75 |
| 1122 | 17.76 |
| 1126 | 26.14 |
| 1127 | 33.37 |
| 1128 | 25.15 |
| 1130 | 25.87 |
| 1131 | 25.30 |
| 1140 | 24.98 |
| 1149 | 28.42 |
| 1151 | 28.59 |
| 1158 | 25.92 |
| 1169 | 26.54 |
| 1174 | 25.25 |
| 1186 | 27.01 |
| 1580 | 38.19 |
| 1590 | 20.84 |
| 1591 | 30.02 |
| 1592 | 26.09 |
| 1593 | 26.51 |

TABLE IV
Significant QTL detected for each examined property

| | Trait | | | | | |
|---|---|---|---|---|---|---|
| | | LOD | | | | |
| | Marker/Linkage | Score* | Phen %^{‡} | Length/cM | Weight | Dom. |
| Microfibril angle | I14_09-F15_10/E | 2.38* | 39.8 | 37.3 | 0.9445 | 4.4460 |
| Fibre Coarseness | I14_09-F15_10/E | 3.49 | 55.9 | 37.3 | 72.794 | −79.906 |
| Maceration yield | P1258-P75/C | 3.50 | 68.8 | 3.3 | −6.3878 | 6.4285 |
| | I17_04-P1275/J | 3.18 | 75.4 | 15.4 | −5.3740 | 7.8547 |
| | P1218-G02_11/J | 4.26 | 73.4 | 13.8 | −5.7903 | 7.9257 |
| | P1027-P192/R | 2.98 | 50.0 | 0.0 | −2.8721 | 5.7712 |

*LOD—logarithmic odds score;
^{‡}Phen. % - phenotypic variance explained by the QTL detected; length - recombination distance between genetic markers (in centimorgans, cM); weight and dominance measure the comparative effects of the P. trichocarpa and P. deltoides alleles on the phenotype.
*Low significance QTL reported due to location.

Lignin Composition

Data for the lignin compositional analyses undertaken on the core samples are presented in Table V. These phenotypic data were used in a Mapmaker-QTL 1.1 genetic mapping experiment which resulted in the identification of a single, significant QTL for lignin content (shown in Table VI). Due to the extensive industrial and academic interest in the genetic control of this particular woody plant trait, many candidate genes for this region—primarily from the lignin biosynthetic pathway—have already been sequenced, a fact which may enable the rapid characterization of this QTL.

TABLE V
Lignin contents for the harvested stems

| Clone | Lignin (%) |
|---|---|
| 14-129 | 24.56 |
| 93-968 | 25.57 |
| 53-242 | 23.31 |
| 53-246 | 24.50 |
| 331-1059 | 24.89 |
| 331-1061 | 25.75 |
| 331-1062 | 24.78 |
| 331-1075 | 24.87 |
| 331-1093 | 25.43 |
| 331-1118 | 23.99 |
| 331-1122 | 24.27 |
| 331-1126 | 23.38 |
| 331-1136 | 24.56 |
| 331-1162 | 22.93 |
| 331-1186 | 24.71 |

TABLE VI
Significant QTL detected for lignin content

| | Trait | | | | | |
|---|---|---|---|---|---|---|
| | | LOD | | Length/ | | |
| | Marker/Linkage | Score | Phen % | cM | Weight | Dom. |
| Lignin | P757-P867/P | 3.32 | 24.7 | 16.7 | 0.5463 | −0.0099 |
| content | | | | | | |

Extractives Content—GC/MS Analysis

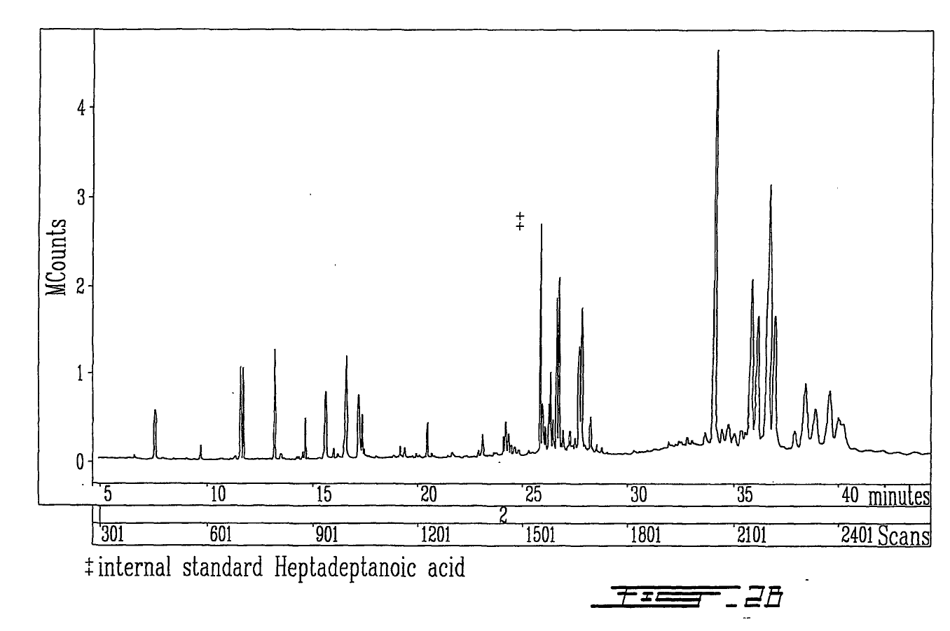

The GCMS method used for compound analysis was that developed and optimized by Fernandez et al. [Fernandez, M. F., Breuil, C., and Watson, P. A. “A gas-chromatography—mass spectrometry method for the simultaneous determination of wood extractive compounds,” Paprican Pulp and Paper Report. In press. (2000)] for the analysis of aspen (P. tremuloides) extractives. Peaks were identified via retention time and ion masses. The area of particular interest in the spectrum—containing the sterols and assorted waxes, compounds which are implicated in pitch formation propensity—was delineated as shown in FIG. 2a, at retention times greater than 25 min. The similarity between this aspen spectrum and those obtained from the hybrid poplar clones—a typical spectrum is shown in FIG. 2b—allowed, the extrapolation of peak identification table data to the mapping population clones. Identified compounds were quantified, ratio numbers were obtained relative to the internal standard and were then used for QTL experiments. Significant QTL for extractives peaks are presented in Table VII.

TABLE VII
Significant QTL detected for individual extractives peaks

| | Trait | | | | | |
|---|---|---|---|---|---|---|
| | | LOD | | | | |
| Compound | Marker/Linkage | Score | Phen % | Length/cM | Weight | Dom. |
| Beta-sitosterol | P1277-P12612/A | 9.84 | 83.3 | 14.7 | 4.7882 | −5.8067 |
| (r.t. 25.831) | P856-A18_06/I | 7.97 | 81.3 | 14.0 | 4.9972 | −5.5280 |
| | win8-G04_20/I | 10.47 | 81.3 | 27.0 | 5.0064 | −5.5178 |
| | P1202-P1221/O | 5.60 | 80.7 | 15.8 | −5.3093 | −4.9808 |
| Sterol | P1277-P12612/A | 5.03 | 69.4 | 14.7 | −0.9132 | −1.1720 |
| (r.t. 25.912) | P1011-C04_04/A | 5.70 | 68.8 | 23.5 | −0.9541 | −1.1478 |
| | P1322-P1310/A | 4.12 | 67.6 | 12.2 | −1.0231 | −0.9421 |
| | P1074-G12_15/B | 5.76 | 65.1 | 19.7 | −1.5614 | −1.4403 |
| | P44-P1054/B | 6.04 | 65.4 | 4.4 | −1.5744 | −1.4237 |
| | H12_03-P1196/B | 3.71 | 58.8 | 8.8 | −1.2545 | −1.0949 |
| | win8-G04_20/I | 5.16 | 64.7 | 27.0 | 1.5482 | −1.4744 |
| | G13_17-C10_21/I | 5.91 | 64.4 | 14.0 | 1.4861 | −1.5144 |
| | P65-P1203/J | 4.86 | 64.6 | 9.1 | 1.5060 | −1.5576 |
| | B15_17-P216/X | 2.97 | 31.5 | 0.4 | −0.5213 | −0.6455 |
| Sterol | win8-G04_20/I | 9.06 | 72.2 | 27.0 | 3.8061 | −3.6553 |
| (r.t. 25.917) | G13_17-C10_21/I | 9.20 | 72.0 | 14.0 | 3.7236 | −3.8242 |
| | I17_04-P1275/J | 8.86 | 72.2 | 15.4 | 3.8034 | −3.6422 |
| | P773-P1055/J | 7.17 | 72.2 | 3.9 | 3.8033 | −3.6495 |
| | P65-P1203/J | 9.21 | 72.0 | 9.1 | 3.7858 | −3.6910 |
| | P1218-G02_11/J | 9.55 | 71.9 | 13.8 | 3.7620 | −3.7391 |
| Sterol | P1277-P12612/A | 12.12 | 90.1 | 14.7 | −0.1879 | −0.3996 |
| (r.t. 26.319) | H19_08-E14_15/C | 6.53 | 81.7 | 19.7 | 0.3026 | −0.2430 |
| | P12182-P1049/C | 5.17 | 75.3 | 19.0 | −0.2181 | −0.2372 |
| | P13292-P1043/M | 6.27 | 79.2 | 12.0 | −0.2791 | −0.2991 |
| | P46-F15_18/X | 8.18 | 80.3 | 17.9 | −0.2996 | −0.2567 |
| | E18_05-P12743/X | 5.00 | 80.3 | 11.5 | −0.3007 | −0.2567 |
| | P1064-B15_17/X | 7.97 | 81.2 | 26.6 | −0.3044 | −0.2468 |
| Sterol/triterpene | P1277-P12612/A | 5.30 | 80.2 | 14.7 | 0.0157 | −0.1606 |
| (r.t. 26.417) | H19_08-E14_15/C | 6.53 | 80.0 | 19.7 | 0.0858 | −0.0726 |
| | P12182-P1049/C | 3.20 | 77.1 | 19.0 | −0.0730 | −0.1091 |
| | P1018-P12242/E | 4.80 | 80.2 | 16.9 | −0.0705 | −0.0957 |
| | P1064-B15_17/X | 3.14 | 80.2 | 26.6 | −0.0782 | −0.0829 |
| Sterol | I14_09-F15_10/E | 3.35 | 65.3 | 37.3 | 0.1014 | −0.0849 |
| (r.t. 27.818) | I17_04-P1275/J | 3.46 | 63.9 | 15.4 | 0.0967 | −0.0985 |
| | P1218-G02_11/J | 4.34 | 63.5 | 13.8 | 0.0959 | −0.1006 |
| | E18_15-C01_16/M | 3.15 | 68.7 | 22.1 | −0.1074 | −0.0778 |
| Sterol/triterpene | P1277-P12612/A | 18.15 | 95.3 | 14.7 | 1.6108 | −1.6355 |
| (r.t. 28.218) | P1011-C04_04/A | 18.99 | 97.3 | 23.5 | 1.5192 | −1.7546 |
| | P1291-P1267/L | 18.13 | 95.5 | 12.9 | 1.5951 | −1.6614 |
| Triterpene/ester | P1145-G08_09/M | 3.96 | 78.4 | 12.7 | −2.6340 | −2.3716 |
| (r.t. 37.833) | E18_15-C01_16/M | 3.76 | 77.1 | 22.1 | 2.5955 | −3.5004 |
| | P1064-B15_17/X | 4.72 | 81.1 | 26.6 | −2.6098 | −3.6878 |
| Triglyceride (r.t. | P11642-P1145/M | 3.13 | 56.3 | 4.5 | −1.3120 | −2.0510 |
| 40.084) | | | | | | |

Compound identification (and retention time, r.t.) as described in the text

To date, this study has successfully identified a number of QTL that putatively contain genes involved in the control of sterol and steryl ester content/synthesis in this family of hybrid poplars. The fact that several QTL have been independently detected for a number of related compounds provides strong evidence that the synthesis of a suite of related compounds is controlled by the same discrete genetic regions (implying the existence of a biosynthetic pathway) and that these QTL in particular may be regarded as non-spurious detections. These results both confirm and extend the conclusions of previous research describing clonal-based variation of extractives content in a natural population of aspen (P. tremuloides).

Chipping and Chip Quality of Hybrid Poplar Stems

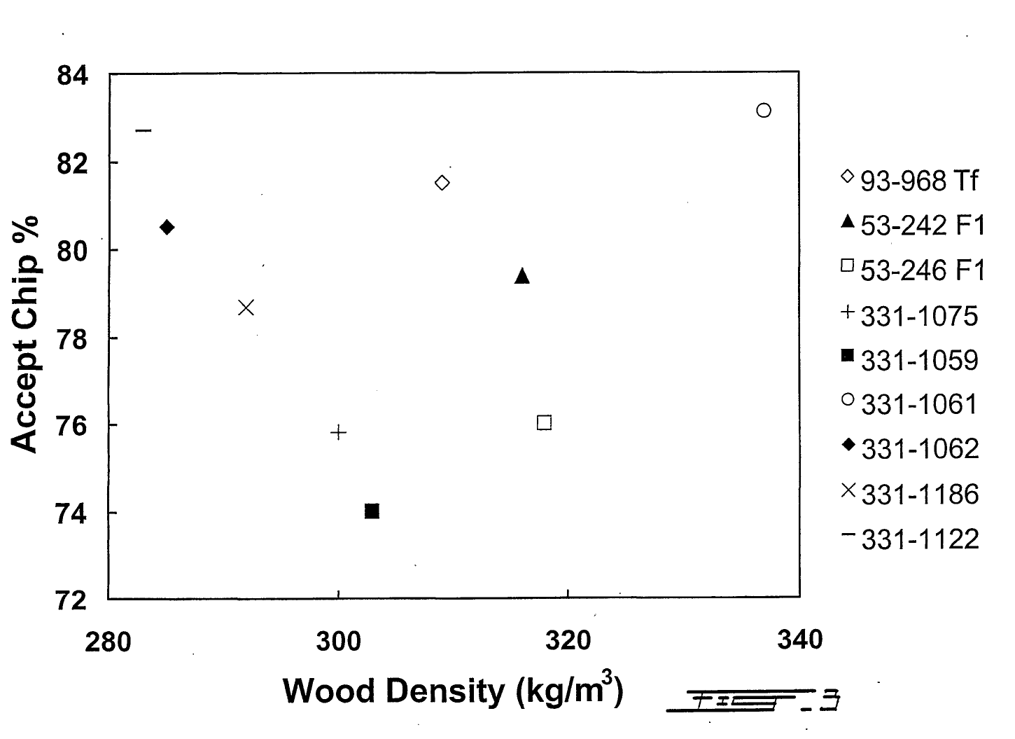

Whole logs of selected hybrid poplar clones were debarked and chipped as described in the experimental section. The wood density and chip quality of selected clones are presented in Table VIII. Attempted correlations between the accept chip fraction and the wood density were unsuccessful (FIG. 3).

TABLE VIII
Wood Density and Chip Quality of Selected Clones

| | 93- | 53- | 53- | 331- | 331- | 331- | 331- | 331- | 331- |
|---|---|---|---|---|---|---|---|---|---|
| | 968 | 242 | 246 | 1059 | 1061 | 1062 | 1075 | 1122 | 1186 |
| Wood | 309 | 316 | 318 | 303 | 337 | 285 | 300 | 283 | 292 |
| Density | | | | | | | | | |
| (kg/m³) | | | | | | | | | |
| 45 mm | 0.9 | 4.3 | 4.5 | 2.9 | 1.4 | 1.4 | 2.9 | 0.2 | 1.8 |
| round | | | | | | | | | |
| (%) | | | | | | | | | |
| 8 mm | 15.2 | 15.1 | 18.4 | 21.8 | 9.8 | 16.5 | 20.0 | 14.2 | 17.1 |
| slot (%) | | | | | | | | | |
| 7 mm | 81.5 | 79.4 | 76.0 | 74.0 | 83.1 | 80.5 | 75.8 | 82.7 | 78.7 |
| round | | | | | | | | | |
| (%) | | | | | | | | | |
| 3 mm | 2.0 | 1.0 | 0.8 | 1.0 | 2.5 | 1.2 | 0.8 | 2.2 | 1.8 |
| round | | | | | | | | | |
| (%) | | | | | | | | | |
| Fines | 0.6 | 0.4 | 0.4 | 0.4 | 0.5 | 0.5 | 0.5 | 0.7 | 0.6 |
| (%) | | | | | | | | | |

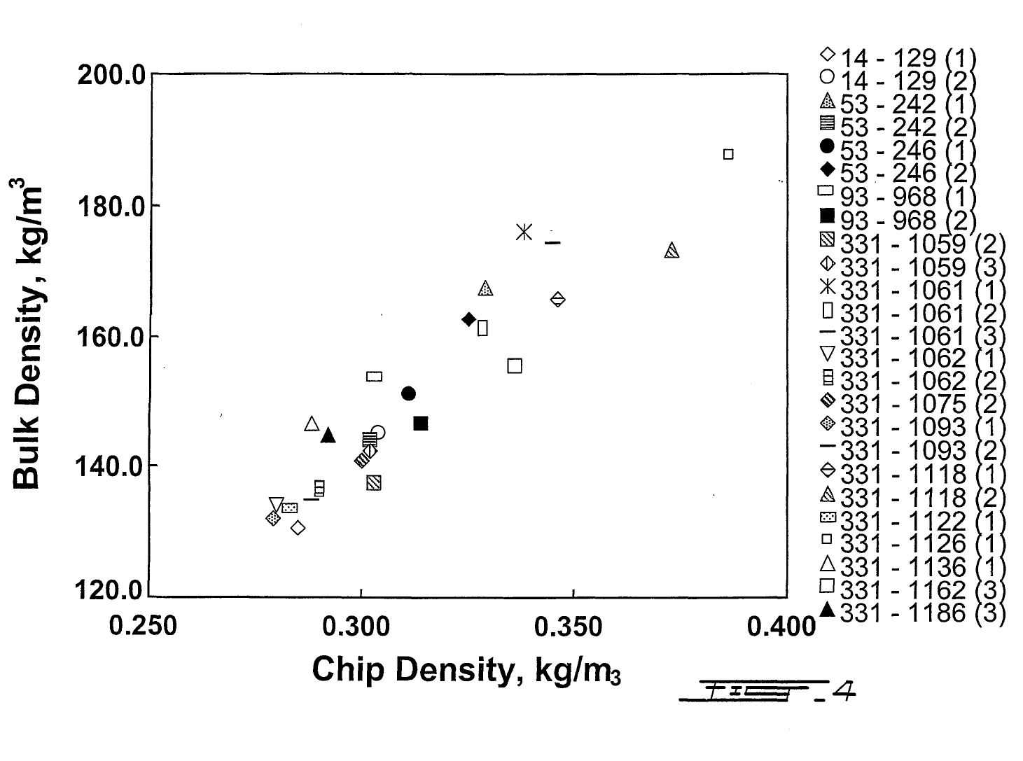

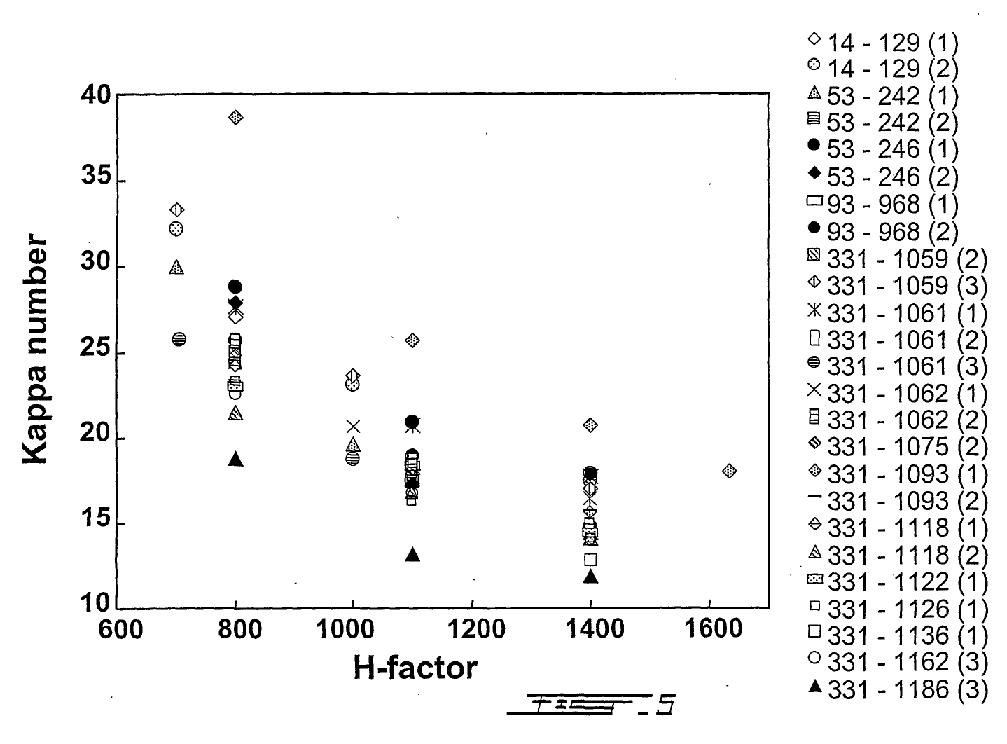

FIG. 4 shows a plot of chip density against bulk density (Table IX) for the sampled stems. The two parameters are related by a Pearson correlation coefficient of 0.86 (p=0.000). Higher density chips, such as those obtained from clone 331-1061, are more desirable as they pack better into kraft pulp digesters and mechanical pulp mill plug screw feeders thus ensuring maximum mill production rates. If these clones were to be ranked on the basis of chip value and quality (i.e. low oversized, pins and fines fractions), clones 331-1061, 331-1122, parent 93-968 and triploid 331-1062 would be considered superior material.

TABLE IX
Hybrid Poplar Chip Density And Chip Packing Density
(Bulk Density) Puyallup, Washington Site

| Sample Air Dried Chips | Chip Density Kg/m³ | Bulk Density Kg/m³ |
|---|---|---|
| 14-129 (1) | 0.285 | 130.7 |
| 14-129 (2) | 0.304 | 145.1 |
| 53-242 (1) | 0.329 | 167.5 |
| 53-242 (2) | 0.302 | 143.9 |
| 53-246 (1) | 0.311 | 151.0 |
| 53-246 (2) | 0.325 | 162.6 |
| 93-968 (1) | 0.303 | 153.3 |
| 93-968 (2) | 0.314 | 146.5 |
| 331-1059 (2) | 0.303 | 137.5 |
| 331-1059 (3) | 0.302 | 142.3 |
| 331-1061 (1) | 0.338 | 176.1 |
| 331-1061 (2) | 0.328 | 161.4 |
| 331-1061 (3) | 0.345 | 174.3 |
| 331-1062 (1) | 0.280 | 133.8 |
| 331-1062 (2) | 0.290 | 136.2 |
| 331-1075 (2) | 0.300 | 140.8 |
| 331-1093 (1) | 0.279 | 132.1 |
| 331-1093 (2) | 0.288 | 134.8 |
| 331-1118 (1) | 0.346 | 165.7 |
| 331-1118 (2) | 0.373 | 173.3 |
| 331-1122 (1) | 0.283 | 133.5 |
| 331-1126 (1) | 0.386 | 188.0 |
| 331-1136 (1) | 0.288 | 146.5 |
| 331-1162 (3) | 0.336 | 155.4 |
| 331-1186 (3) | 0.292 | 144.7 |

Note:
Chip Thickness = 2-6 mm; Bulk density was done on air dried chips

Kraft Pulping and Testing
Pulping Data

The 25 hybrid poplar trees (comprising 15 distinct genotypes) were chemically pulped according to the conditions outlined above and handsheets were prepared from the corresponding pulps. Calculated data for pulping to Kappa 17, derived from Table IX, are presented in Table X.

TABLE X
Kraft pulping data for harvested stems (Kappa 17)

| | H-Factor | Unscreened Yield (%) | % EA Consumed |
|---|---|---|---|
| 14-129 | 1230 | 54.4 | 10.3 |
| | 1436 | 53.5 | 10.9 |
| 93-968 | 1110 | 56.5 | 10.5 |
| | 883 | 58.0 | 10.0 |
| 53-242 | 1092 | 54.7 | 10.7 |
| | 1211 | 55.4 | 10.6 |
| 53-246 | 1112 | 54.7 | 10.6 |
| | 1088 | 55.9 | 10.5 |
| 331-1059 | 1213 | 54.4 | 10.8 |
| | 1190 | 54.0 | 10.9 |
| 331-1061 | 1448 | 52.9 | 11.0 |
| | 1200 | 53.9 | 10.8 |
| | 1225 | 54.0 | 10.8 |
| 331-1062 | 1219 | 53.3 | 10.8 |
| | 1207 | 52.9 | 10.8 |
| 331-1075 | 1401 | 53.0 | 10.9 |
| 331-1093 | 1443 | 52.8 | 10.9 |
| | 1236 | 52.9 | 10.8 |
| 331-1118 | 1135 | 55.3 | 10.6 |
| | 1251 | 54.5 | 10.6 |
| 331-1122 | 1206 | 53.6 | 10.8 |
| 331-1126 | 1177 | 54.4 | 10.5 |
| 331-1136 | 1684 | 51.1 | 11.3 |
| 331-1162 | 1132 | 53.4 | 10.4 |
| 331-1186 | 1146 | 54.7 | 10.6 |

Bold = top yielding clones

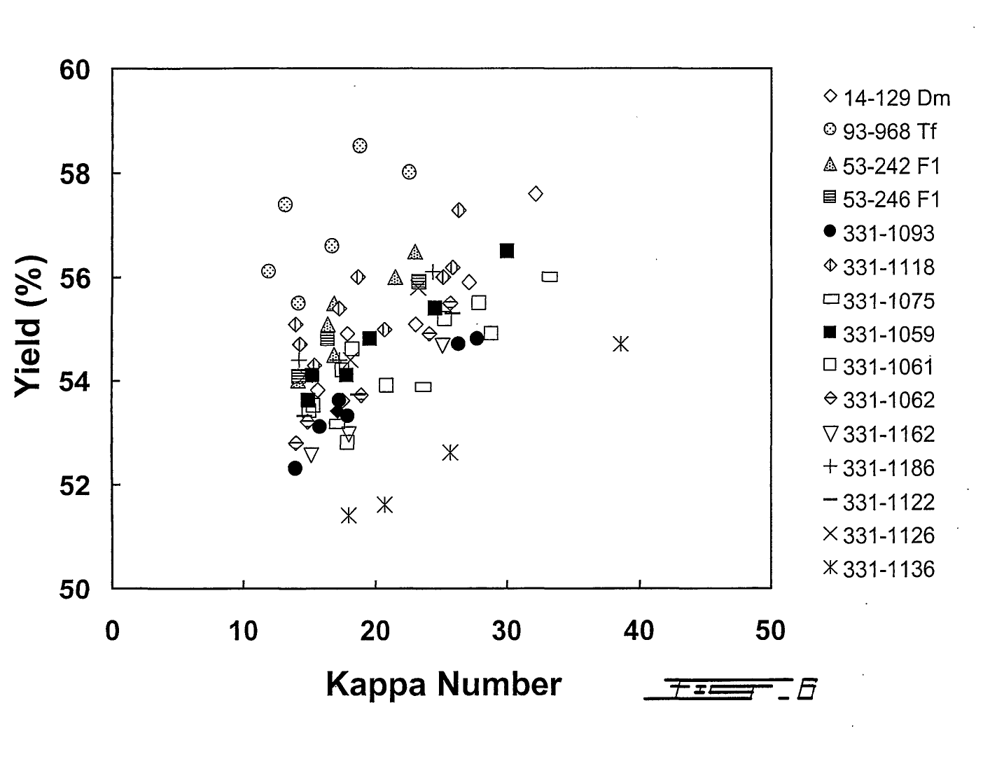

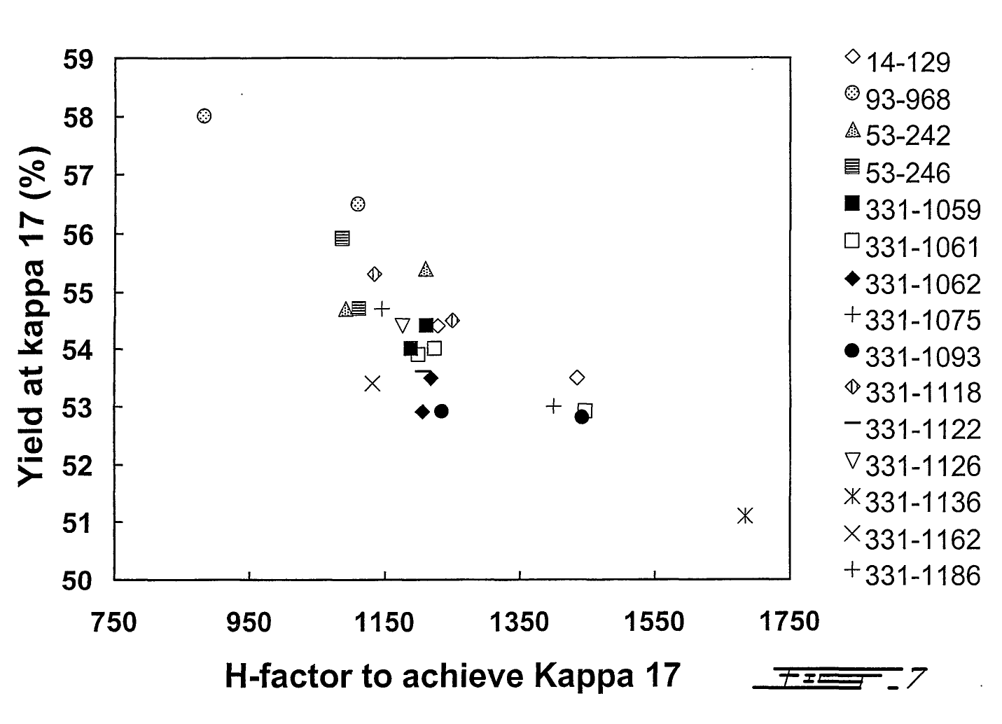

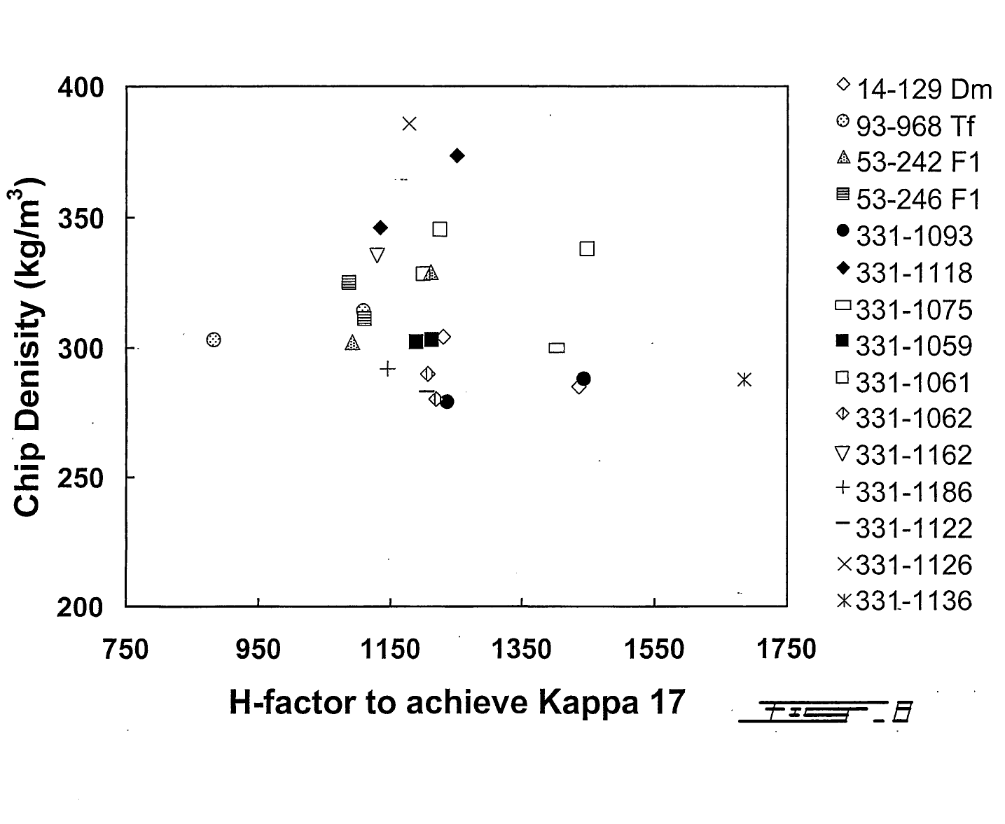

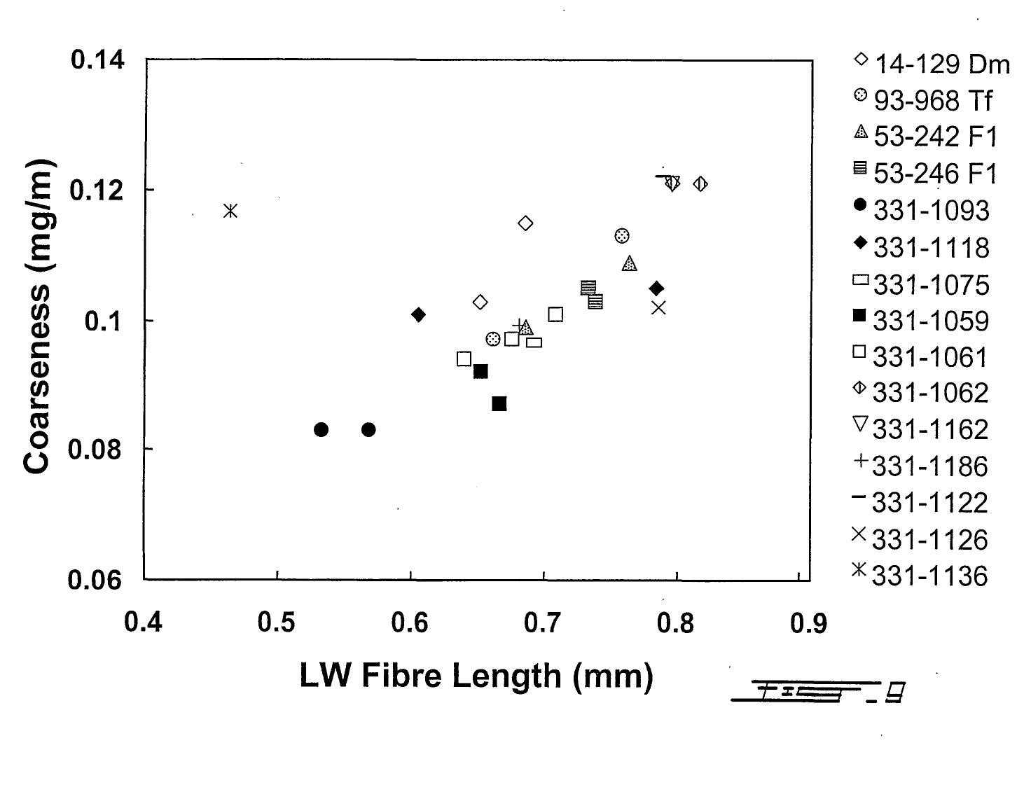

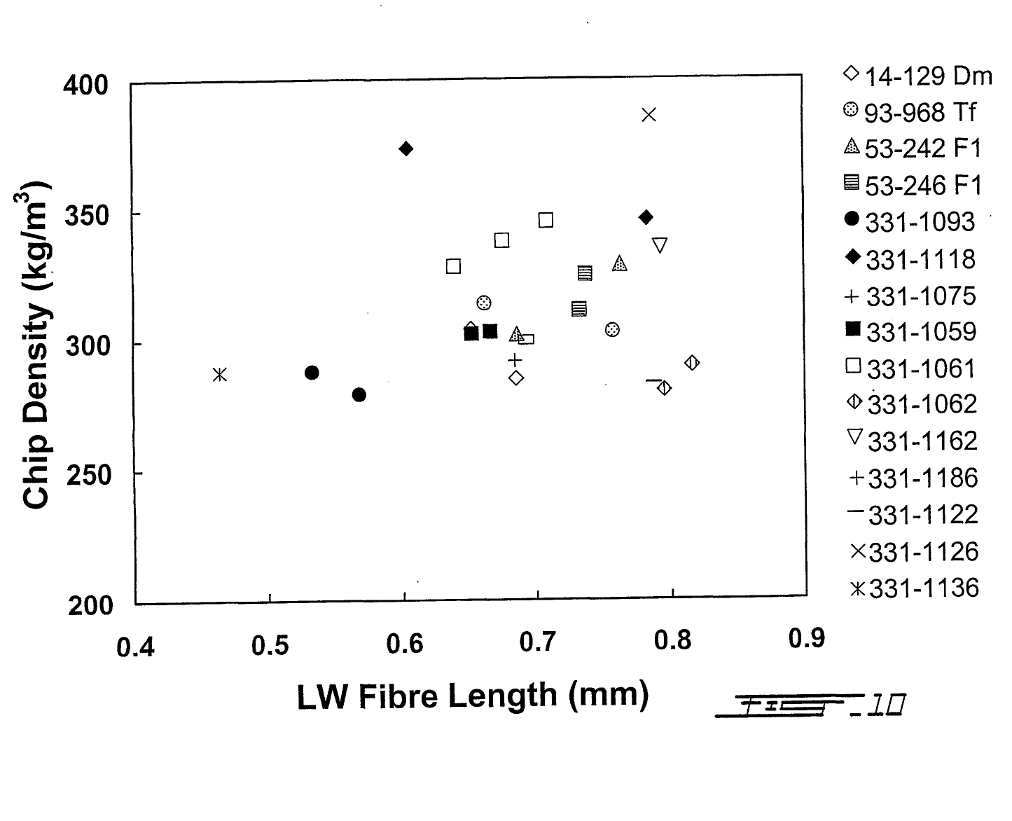

FIG. 5 shows the relationship between H-factor and Kappa number for the pulped stems. It is clear that, as was the case for aspen, the variation in H-factor required to achieve a given Kappa number is substantial. For example, to achieve Kappa 17, clone 331-1136 requires approximately 1650 H-factor whereas clone 93-968 requires only 1000 H-factor (a 40% reduction. The particular difficulty in pulping clone 331-1136 indicated here may be a function of this clone's high level of calcium accumulation (see below), particularly as this clone's lignin content is not unusually high (24.56% in a population range of 22.93-25.75%, see Table V). Also like aspen the swings in yield at a given unbleached kappa number are substantial. All the exploratory kraft pulping data are presented in Table XI. At kappa 17 the yield from clone 331-1136 was approximately 51%. This may be an outlier point (excess compression wood due to plantation location, etc.). The lower limit of pulp yield is probably better represented by clones 331-1093 and 331-1062 whereas clone 93-968 exhibits a 57% pulp yield (FIG. 6). Superior clones are highlighted in Table X. The relationship between ease of pulping and pulp yield is evident (Pearson correlation of −0.828, p=0.000). However it should be noted that the variability in yield at a given H-factor is high as evidenced by the relatively poor R² of 0.69, shown in FIG. 7. In FIG. 7, it can be seen that the Parental clones represent the extremes, (clonal lignin content 25.75-22.93%) 331-1162 has the lowest lignin content but gives low pulp yield and average pulping rate, therefore lignin content is not a reliable indicator of pulpability. These results confirm the necessity to pilot pulp clones for proper evaluation of properties. Further, the H-factor required to achieve kappa 17 has been evaluated against the chip density in FIG. 8. It is clear that in addition to lignin content wood density cannot be used to predict ease of kraft pulping (Pearson coefficient −0.194, p=1.000).

TABLE XI
Hybrid Poplar Exploratory Kraft Pulping Data
(whole log chip samples)

| | | % | | | | |
|---|---|---|---|---|---|---|
| | | Unsc'd | H | % Res. | % EA | % |
| Sample | Kappa | Yield | Factor | EA | Consumed | Rejects |
| 14-129(1) | 27.1 | 55.9 | 800 | 3.0 | 10.0 | 0.7 |
| | 17.9 | 54.9 | 1100 | 2.8 | 10.2 | trace |
| | 15.6 | 53.8 | 1400 | 2.5 | 10.5 | 0.1 |
| 14-129(2) | 32.2 | 57.6 | 700 | 3.1 | 9.9 | 4.7 |
| | 23.1 | 55.1 | 1000 | 2.6 | 10.4 | 1.1 |
| | 17.5 | 53.6 | 1400 | 2.2 | 10.8 | 0.1 |
| 331-1059(2) | 30.0 | 56.5 | 700 | 2.7 | 10.3 | 3.2 |
| | 19.6 | 54.8 | 1000 | 2.3 | 10.7 | 0.3 |
| | 15.2 | 54.1 | 1400 | 2.1 | 10.9 | 0.2 |
| 331-1059(3) | 24.6 | 55.4 | 800 | 2.5 | 10.5 | 0.4 |
| | 17.8 | 54.1 | 1100 | 2.2 | 10.8 | 0.2 |
| | 14.9 | 53.6 | 1400 | 2.0 | 11.0 | 0.4 |
| 331-1061(1) | 28.8 | 54.9 | 800 | 2.4 | 10.6 | 1.0 |
| | 20.9 | 53.9 | 1100 | 2.2 | 10.8 | 0.1 |
| | 17.9 | 52.8 | 1400 | 2.0 | 11.0 | trace |
| 331-1061(2) | 27.9 | 55.5 | 800 | 2.5 | 10.5 | 1.5 |
| | 17.5 | 54.2 | 1100 | 2.3 | 10.7 | trace |
| | 15.0 | 53.4 | 1400 | 2.1 | 10.9 | trace |
| 331-1061(3) | 25.3 | 55.2 | 800 | 2.5 | 10.5 | 0.5 |
| | 18.3 | 54.6 | 1100 | 2.3 | 10.7 | 0.2 |
| | 15.3 | 53.5 | 1400 | 2.0 | 11.0 | 0.3 |
| 331-1062(1) | 25.7 | 55.5 | 800 | 2.7 | 10.3 | 2.4 |
| | 18.9 | 53.7 | 1100 | 2.3 | 10.7 | 0.5 |
| | 14.8 | 53.2 | 1400 | 2.1 | 10.9 | trace |
| 331-1062(2) | 25.2 | 54.6 | 800 | 2.5 | 10.5 | 0.9 |
| | 18.0 | 53.0 | 1100 | 2.2 | 10.8 | 0.4 |
| | 15.1 | 52.6 | 1400 | 2.1 | 10.9 | trace |
| 331-1075(2) | 33.3 | 56.0 | 700 | 2.7 | 10.3 | 5.4 |
| | 23.6 | 53.9 | 1000 | 2.4 | 10.6 | 0.7 |
| | 17.0 | 53.2 | 1400 | 2.1 | 10.9 | 0.5 |
| 331-1093(1) | 27.7 | 54.8 | 800 | 2.6 | 10.4 | 1.7 |
| | 20.7 | 53.3 | 1100 | 2.3 | 10.7 | 0.4 |
| | 17.7 | 53.1 | 1400 | 2.2 | 10.8 | 0.5 |
| 331-1093(2) | 25.7 | 54.7 | 800 | 2.6 | 10.4 | 1.0 |
| | 17.9 | 53.6 | 1100 | 2.3 | 10.7 | 0.4 |
| | 15.8 | 52.3 | 1400 | 2.0 | 11.0 | trace |
| 331-1118(1) | 25.8 | 56.2 | 705 | 2.8 | 10.2 | 1.3 |
| | 18.7 | 56.0 | 1000 | 2.6 | 10.4 | 0.4 |
| | 14.3 | 54.7 | 1400 | 2.2 | 10.8 | 0.1 |
| 331-1118(2) | 25.1 | 56.0 | 800 | 2.8 | 10.2 | 1.3 |
| | 20.7 | 55.0 | 1000 | 2.5 | 10.5 | 0.4 |
| | 15.4 | 54.3 | 1400 | 2.3 | 10.7 | 0.1 |
| 331-1122(1) | 25.8 | 55.3 | 800 | 2.5 | 10.5 | 1.6 |
| | 18.7 | 53.7 | 1100 | 2.2 | 10.8 | 0.1 |
| | 14.6 | 53.3 | 1400 | 2.1 | 10.9 | 0.1 |
| 331-1126(1) | 23.2 | 55.8 | 800 | 2.8 | 10.2 | 1.1 |
| | 18.1 | 54.4 | 1100 | 2.5 | 10.5 | 0.1 |
| | 14.7 | 54.1 | 1400 | 2.3 | 10.7 | trace |
| 331-1136(1) | 38.6 | 54.7 | 800 | 2.1 | 10.9 | 5.4 |
| | 25.7 | 52.6 | 1100 | 1.9 | 12.1 | 1.7 |
| | 20.7 | 51.6 | 1400 | 1.8 | 12.2 | 1.1 |
| | 18.0 | 51.4 | 1634 | 1.7 | 11.3 | na |
| 331-1162(3) | 24.1 | 54.9 | 800 | 2.9 | 10.1 | 0.5 |
| | 17.1 | 53.4 | 1100 | 2.6 | 10.4 | trace |
| | 14.0 | 52.8 | 1400 | 2.4 | 10.6 | trace |
| 331-1186(3) | 24.3 | 56.1 | 800 | 2.7 | 10.3 | 0.6 |
| | 17.3 | 54.4 | 1100 | 2.4 | 10.6 | trace |
| | 14.2 | 54.4 | 1400 | 2.2 | 10.8 | 0.1 |
| 53-242(1) | 21.5 | 56.0 | 800 | 2.6 | 10.4 | 0.8 |
| | 16.9 | 54.5 | 1100 | 2.3 | 10.7 | 0.2 |
| | 14.1 | 54.0 | 1400 | 2.1 | 10.9 | trace |
| 53-242(2) | 23.0 | 56.5 | 800 | 2.7 | 10.3 | 2.7 |
| | 16.9 | 55.5 | 1100 | 2.5 | 10.5 | 1.0 |
| | 16.4 | 55.1 | 1400 | 2.3 | 10.7 | 2.4 |
| 53-246(1) | 23.3 | 55.9 | 800 | 2.7 | 10.3 | 1.4 |
| | 16.4 | 54.8 | 1100 | 2.4 | 10.6 | 0.2 |
| | 14.2 | 54.0 | 1400 | 2.2 | 10.8 | trace |
| 53-246(2) | 23.1 | 56.6 | 800 | 2.7 | 10.3 | 1.0 |
| | 17.4 | 56.1 | 1100 | 2.5 | 10.5 | 0.9 |
| | 12.8 | 55.2 | 1400 | 2.4 | 10.6 | trace |
| 93-968(1) | 22.6 | 58.0 | 800 | 2.8 | 10.2 | 2.4 |
| | 16.7 | 56.6 | 1100 | 2.6 | 10.4 | 0.2 |
| | 14.2 | 55.5 | 1400 | 2.2 | 10.8 | trace |
| 93-968(2) | 18.8 | 58.5 | 800 | 3.1 | 9.9 | 0.9 |
| | 13.2 | 57.4 | 1100 | 2.8 | 10.2 | 0.1 |
| | 11.9 | 56.1 | 1400 | 2.5 | 10.5 | trace |

Table XII presents the fibre properties data obtained for the pulped clones at Kappa 17.

TABLE XII
Whole stem pulp fibre properties data

| | LW Fibre Length (mm) | Coarseness (mg/m) |
|---|---|---|
| 14-129 | 0.65 | 0.103 |
| | 0.69 | 0.115 |
| 93-968 | 0.66 | 0.097 |
| | 0.76 | 0.113 |
| 53-242 | 0.69 | 0.099 |
| | 0.76 | 0.109 |
| 53-246 | 0.73 | 0.105 |
| | 0.74 | 0.103 |
| 331-1059 | 0.67 | 0.087 |
| | 0.65 | 0.092 |
| 331-1061 | 0.68 | 0.097 |
| | 0.64 | 0.094 |
| | 0.71 | 0.101 |
| 331-1062 | 0.80 | 0.121 |
| | 0.82 | 0.121 |
| 331-1075 | 0.69 | 0.097 |
| 331-1093 | 0.53 | 0.083 |
| | 0.57 | 0.083 |
| 331-1118 | 0.78 | 0.105 |
| | 0.61 | 0.101 |
| 331-1122 | 0.79 | 0.122 |
| 331-1126 | 0.79 | 0.102 |
| 331-1136 | 0.46 | 0.117 |
| 331-1162 | 0.80 | 0.121 |
| 331-1186 | 0.68 | 0.099 |

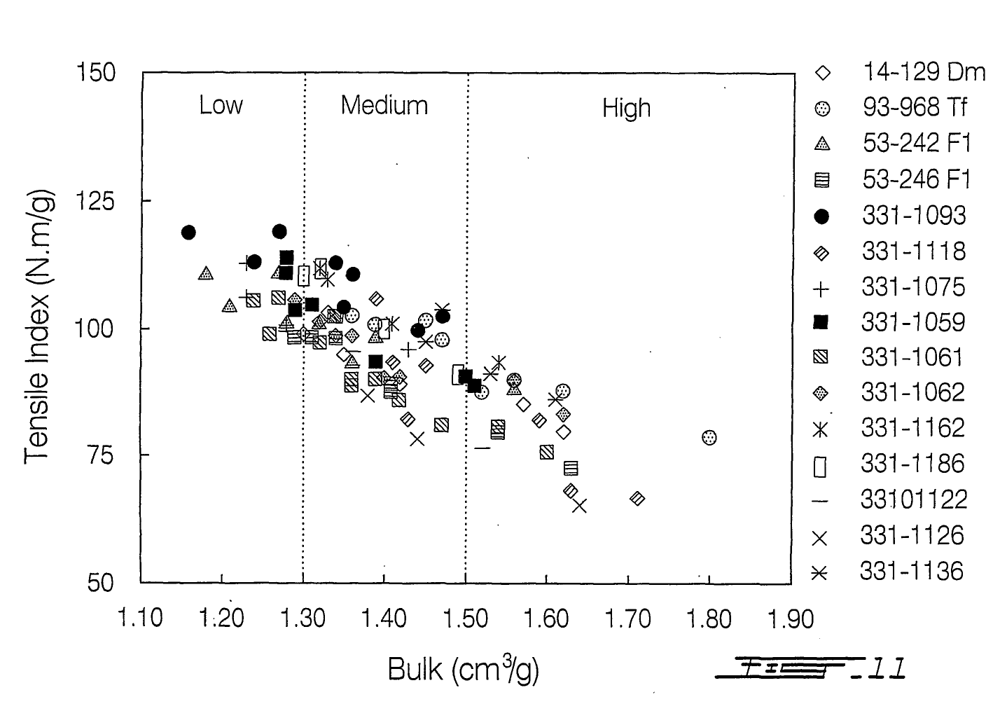

A positive correlation (Pearson coefficient 0.543, p=0.105) can be seen between the fibre length and coarseness data which mirrors that seen for the 7^{th} year ring data and the situation seen in aspen populations (FIG. 9). If the outlier point for clone 331-1136 is omitted from the analysis, the correlation becomes much more significant (Pearson coefficient 0.834, p=0.000).

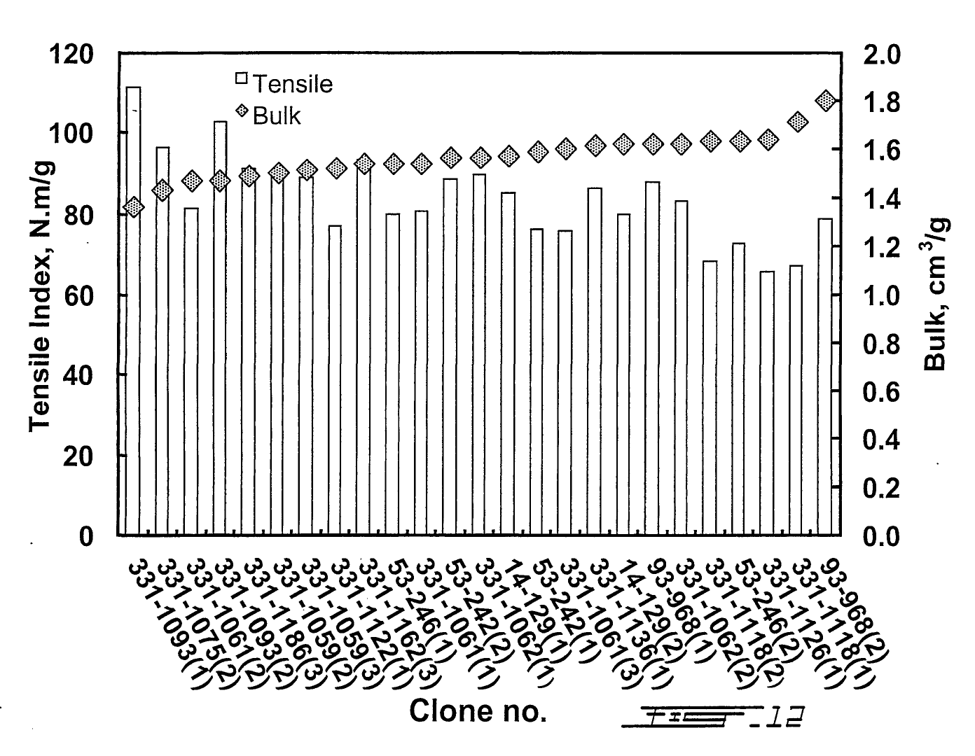

The length-weighted fibre length data were also correlated to chip density values, as shown in FIG. 10. Not unexpectedly, and bearing in mind the fibre length: coarseness relationship, the relationship is poor (Pearson coefficient 0.228, p=1.000) even if outlier points are excluded.

Pulp yield data at kappa 17, Table X, were used in a Mapmaker-QTL 1.1 analysis which revealed the presence of a single, low significance QTL for this property (Table XIII). The pilot-scale pulping of further clones will likely enhance the statistical significance of the detection of this QTL. Significantly, the QTL kraft pulp yield (the most important trait from an industrial production point of view) correlate with a higher significance QTL for maceration yield but does not coincide with the lignin QTL (Table VI).

TABLE XIII
Low significance QTL detected for Kraft pulp yield

| | Trait | | | | | |
|---|---|---|---|---|---|---|
| | | LOD | Phen | Length/ | | |
| | Marker/Linkage | Score | % | cM | Weight | Dom. |
| Kraft pulp | P1027-P192/R | 2.52* | 72.7 | 0.0 | −1.8932 | 0.7270 |
| yield | | | | | | |

H-factor to kappa 17 data from Table XI were also used in a Mapmaker QTL1.1 analysis. However, no significant QTLs were observed which confirms that lignin content is not the single controlling factor in kraft pulping of hybrid poplar. There may be concern that this observation does not seem to relate to measurable physical properties. However, issues such as pulping liquor diffusion are also known to be a major contributor to ease of kraft pulping.

Kraft Pulp Properties

Kraft Pulp Strengths

The strength of hardwood pulps is becoming an increasingly important parameter given the economic impetus for lighter weight products which retain strength and optical properties and to reduce the amount of expensive softwood Kraft pulp required for many paper grades. Four point PFI mill beater curves were developed for each of the clonal pulps and the results of all tests are presented in Table XIV.

TABLE XIV
Hybrid poplar kraft pulp and optical property data

| | 14-129 (1) | | | | 14-129 (2) | | | | 331-1059 (2) | | | |
|---|---|---|---|---|---|---|---|---|---|---|---|---|
| PFI Revolutions | 0 | 1000 | 3000 | 6000 | 0 | 1000 | 3000 | 6000 | 0 | 1000 | 3000 | 6000 |
| Screened Csf (mL) | 499 | 480 | 414 | 361 | 533 | 479 | 423 | 353 | 453 | 435 | 362 | 322 |
| Apparent Density (kg/m³) | 636 | 703 | 739 | 754 | 618 | 705 | 740 | 767 | 666 | 775 | 784 | 784 |
| Burst Index (kPa · m²/g) | 4.7 | 6.2 | 7.0 | 7.6 | 4.2 | 5.8 | 6.6 | 7.1 | 6.1 | 7.9 | 8.8 | 9.5 |
| Breaking length (km) | 8.7 | 9.3 | 10.6 | 10.5 | 8.2 | 9.1 | 9.7 | 10.1 | 9.3 | 10.6 | 11.3 | 11.6 |
| Tensile Index (N · m/g) | 85.1 | 90.9 | 104.1 | 103.4 | 79.9 | 89.2 | 95.1 | 99.2 | 90.9 | 104.0 | 111.1 | 113.9 |
| Stretch (%) | 1.58 | 2.58 | 3.44 | 3.68 | 1.60 | 2.71 | 2.97 | 3.55 | 3.11 | 4.46 | 5.01 | 5.26 |
| Tear Index (mN · m²/g) (1 Ply) | 6.0 | 7.2 | 7.5 | 7.9 | 5.6 | 6.6 | 7.1. | 6.7 | 8.3 | 9.4 | 9.0 | 9.0 |
| Tear Index (mN · m²/g) (4 Ply) | 7.2 | 7.6 | 7.6 | 7.5 | 7.7 | 7.4 | 7.6 | 7.4 | 8.7 | 9.1 | 9.0 | 8.6 |
| Zero Span Breaking Length (km) | 15.9 | 15.1 | 15.8 | 15.5 | 15.3 | 15.6 | 16.3 | 16.0 | 14.0 | 13.4 | 13.4 | 12.8 |
| Air Resistance (Gurley) | 65.0 | 121.5 | 206.8 | 372.4 | 42.0 | 85.4 | 133.4 | 292.8 | 130.6 | 249.6 | 476.2 | 862.1 |
| (sec/100 mL) | | | | | | | | | | | | |
| Sheffield Roughness (mL/min) | 89 | 52 | 40 | 27 | 107 | 68 | 52 | 33 | 61 | 31 | 22 | 17 |
| Brightness | 37 | | | | 37 | | | | 38 | | | |
| Opacity (%) | 96.0 | 95.9 | 94.4 | 93.0 | 97.3 | 96.1 | 93.9 | 92.3 | 96.8 | 95.2 | 94.0 | 92.1 |
| Scattering Coefficient (cm²/g) | 311 | 289 | 258 | 229 | 338 | 286 | 242 | 211 | 327 | 266 | 221 | 197 |
| | 331-1059 (3) | | | | 331-1061(1) | | | | 331-1061(2) | | | |
| PFI Revolutions | 0 | 1000 | 3000 | 6000 | 0 | 1000 | 3000 | 6000 | 0 | 1000 | 3000 | 6000 |
| Screened Csf (mL) | 486 | 454 | 372 | 339 | 524 | 478 | 395 | 346 | 524 | 478 | 395 | 346 |
| Apparent Density (kg/m³) | 663 | 717 | 757 | 765 | 648 | 721 | 755 | 786 | 682 | 734 | 793 | 807 |
| Burst Index (kPa · m²/g) | 6.1 | 7.5 | 8.2 | 8.7 | 4.9 | 6.1 | 6.9 | 7.3 | 4.9 | 6.5 | 7.6 | 8.1 |
| Breaking length (km) | 9.1 | 9.6 | 9.9 | 10.7 | 8.2 | 9.2 | 9.9 | 10.8 | 8.3 | 9.2 | 10.1 | 10.8 |
| Tensile Index (N · m/g) | 88.8 | 93.7 | 97.3 | 105.0 | 80.7 | 90.3 | 97.4 | 106.1 | 81.2 | 90.3 | 99.0 | 105.8 |
| Stretch (%) | 2.78 | 3.91 | 4.77 | 7.95 | 1.96 | 2.90 | 3.45 | 4.25 | 1.99 | 3.35 | 3.73 | 4.45 |
| Tear Index (mN · m²/g) (1 Ply) | 7.5 | 8.9 | 8.9 | 9.0 | 7.9 | 8.8 | 8.4 | 8.7 | 6.2 | 8.0 | 7.8 | 8.0 |
| Tear Index (mN · m²/g) (4 Ply) | 8.2 | 8.3 | 8.4 | 8.4 | 8.2 | 8.2 | 8.5 | 8.5 | 7.9 | 8.3 | 8.6 | 8.1 |
| Zero Span Breaking Length (km) | 14.7 | 13.9 | 14.0 | 13.7 | 16.3 | 15.2 | 15.0 | 13.7 | 15.8 | 16.1 | 16.2 | 16.0 |
| Air Resistance (Gurley) | 119.8 | 177.4 | 325.0 | 537.0 | 75.8 | 147.3 | 219.6 | 449.7 | 55.1 | 101.1 | 201.0 | 359.9 |
| (sec/100 mL) | | | | | | | | | | | | |
| Sheffield Roughness (mL/min) | 62 | 40 | 30 | 23 | 79 | 53 | 41 | 27 | 87 | 59 | 37 | 26 |
| Brightness | 37 | | | | 35 | | | | 38 | | | |
| Opacity (%) | 96.5 | 95.0 | 93.0 | 91.5 | 96.4 | 93.9 | 93.0 | 91.9 | 95.4 | 94.5 | 93.1 | 91.4 |
| Scattering Coefficient (cm²/g) | 323 | 253 | 214 | 193 | 298 | 243 | 222 | 200 | 305 | 269 | 232 | 212 |
| | 331-1061(3) | | | | 331-1062(1) | | | | 331-1062(2) | | | |
| PFI Revolutions | 0 | 1000 | 3000 | 6000 | 0 | 1000 | 3000 | 6000 | 0 | 1000 | 3000 | 6000 |
| Screened Csf (mL) | 552 | 492 | 420 | 353 | 554 | 536 | 469 | 412 | 561 | 527 | 466 | 397 |
| Apparent Density (kg/m³) | 625 | 705 | 736 | 748 | 642 | 716 | 745 | 775 | 619 | 702 | 735 | 757 |
| Burst Index (kPa · m²/g) | 4.3 | 6.1 | 7.1 | 7.4 | 4.9 | 6.1 | 7.1 | 7.6 | 4.9 | 6.2 | 6.7 | 7.6 |
| Breaking length (km) | 7.7 | 8.8 | 9.0 | 10.5 | 9.2 | 9.2 | 10.1 | 10.8 | 8.5 | 9.3 | 10.1 | 10.4 |
| Tensile Index (N · m/g) | 75.9 | 86.0 | 88.7 | 102.6 | 89.8 | 90.6 | 98.9 | 106.0 | 83.3 | 90.9 | 98.9 | 101.7 |
| Stretch (%) | 1.69 | 2.91 | 3.10 | 4.13 | 1.98 | 2.69 | 3.44 | 3.88 | 1.66 | 2.83 | 3.39 | 3.45 |
| Tear Index (mN · m²/g) (1 Ply) | 6.2 | 9.0 | 8.6 | 9.2 | 8.6 | 8.7 | 8.6 | 8.5 | 7.2 | 7.2 | 7.8 | 8.4 |
| Tear Index (mN · m²/g) (4 Ply) | 8.2 | 9.3 | 9.0 | 9.0 | 8.9 | 8.7 | 8.5 | 8.2 | 8.7 | 8.9 | 8.5 | 8.2 |
| Zero Span Breaking Length (km) | 15.9 | 16.3 | 15.2 | 14.2 | 17.6 | 17.0 | 15.7 | 15.8 | 15.2 | 15.0 | 15.0 | 15.3 |
| Air Resistance (Gurley) | 28.4 | 74.8 | 140.6 | 234.1 | 72.5 | 148.7 | 279.1 | 562.1 | 51.7 | 115.6 | 210.5 | 412.4 |
| (sec/100 mL) | | | | | | | | | | | | |
| Sheffield Roughness (mL/min) | 115 | 76 | 55 | 39 | 87 | 55 | 38 | 27 | 109 | 68 | 43 | 28 |
| Brightness | 37 | | | | 36 | | | | 37 | | | |
| Opacity (%) | 95.2 | 93.4 | 92.2 | 90.9 | 94.9 | 93.0 | 91.6 | 89.3 | 95.2 | 92.5 | 90.8 | 89.2 |
| Scattering Coefficient (cm²/g) | 304 | 254 | 229 | 204 | 268 | 221 | 193 | 167 | 286 | 233 | 201 | 179 |
| | 331-1075(2) | | | | 331-1093(1) | | | | 331-1093(2) | | | |
| PFI Revolutions | 0 | 1000 | 3000 | 6000 | 0 | 1000 | 3000 | 6000 | 0 | 1000 | 3000 | 6000 |
| Screened Csf (mL) | 483 | 451 | 375 | 328 | 405 | 393 | 336 | 298 | 425 | 403 | 354 | 294 |
| Apparent Density (kg/m³) | 701 | 781 | 813 | 816 | 734 | 807 | 789 | 861 | 679 | 696 | 742 | 749 |
| Burst Index (kPa · m²/g) | 6.2 | 7.5 | 8.0 | 8.5 | 7.0 | 8.0 | 8.7 | 9.4 | 6.3 | 7.7 | 8.1 | 8.8 |
| Breaking length (km) | 9.8 | 10.3 | 10.9 | 11.5 | 11.3 | 11.6 | 12.2 | 12.1 | 10.5 | 10.2 | 10.7 | 11.5 |
| Tensile Index (N · m/g) | 96.2 | 101.3 | 106.5 | 113.1 | 111.2 | 113.5 | 119.2 | 119.0 | 102.6 | 99.8 | 104.8 | 113.2 |
| Stretch (%) | 2.58 | 3.41 | 3.97 | 4.73 | 2.53 | 3.77 | 4.35 | 4.61 | 2.71 | 3.42 | 3.89 | 4.80 |
| Tear Index (mN · m²/g) (1 Ply) | 8.1 | 7.9 | 8.1 | 7.8 | 6.7 | 7.5 | 7.7 | 7.8 | 8.6 | 7.8 | 8.4 | 8.4 |
| Tear Index (mN · m²/g) (4 Ply) | 9.0 | 9.1 | 8.5 | 8.3 | 8.3 | 7.9 | 8.0 | 7.4 | 8.2 | 8.0 | 8.1 | 8.0 |
| Zero Span Breaking Length (km) | 16.3 | 14.7 | 14.3 | 13.2 | 15.0 | 14.7 | 14.3 | 13.8 | 15.7 | 15.1 | 14.5 | 14.1 |
| Air Resistance (Gurley) | 105.9 | 281.4 | 510.0 | 1152.7 | 274.7 | 409.6 | 719.4 | 1351.2 | 202.8 | 527.0 | 802.0 | 1378.1 |
| (sec/100 mL) | | | | | | | | | | | | |
| Sheffield Roughness (mL/min) | 61 | 34 | 22 | 15 | 37 | 25 | 17 | 13 | 46 | 25 | 16 | 10 |
| Brightness | 35 | | | | 38 | | | | 38 | | | |
| Opacity (%) | 95.3 | 93.0 | 91.8 | 89.0 | 96.1 | 94.2 | 92.4 | 91.0 | 96.1 | 94.0 | 92.8 | 90.3 |
| Scattering Coefficient (cm²/g) | 287 | 224 | 201 | 169 | 318 | 260 | 230 | 204 | 323 | 260 | 233 | 203 |
| | 331-1118(1) | | | | 331-1118(2) | | | | 331-1122(1) | | | |
| PFI Revolutions | 0 | 1000 | 3000 | 6000 | 0 | 1000 | 3000 | 6000 | 0 | 1000 | 3000 | 6000 |
| Screened Csf (mL) | 532 | 487 | 401 | 344 | 573 | 538 | 499 | 443 | 553 | 493 | 453 | 406 |
| Apparent Density (kg/m³) | 585 | 628 | 692 | 708 | 613 | 701 | 734 | 722 | 660 | 734 | 737 | 780 |
| Burst Index (kPa · m²/g) | 4.3 | 5.8 | 6.8 | 7.5 | 4.0 | 6.1 | 6.8 | 7.9 | 4.6 | 6.1 | 6.9 | 7.4 |
| Breaking length (km) | 6.9 | 8.4 | 9.5 | 9.5 | 7.0 | 8.4 | 9.1 | 10.8 | 7.8 | 9.5 | 9.8 | 10.2 |
| Tensile Index (N · m/g) | 67.2 | 82.1 | 92.8 | 93.5 | 68.4 | 82.5 | 89.6 | 106.1 | 76.7 | 92.8 | 95.7 | 99.6 |
| Stretch (%) | 2.42 | 3.86 | 4.67 | 4.74 | 2.01 | 3.22 | 4.24 | 4.80 | 1.68 | 3.07 | 3.52 | 3.82 |
| Tear Index (mN · m²/g) (1 Ply) | 7.1 | 8.6 | 8.6 | 9.1 | 6.6 | 8.6 | 9.5 | 10.5 | 7.3 | 8.8 | 8.7 | 8.4 |
| Tear Index (mN · m²/g) (4 Ply) | 8.6 | 8.4 | 8.7 | 8.8 | 8.6 | 9.4 | 9.5 | 10.1 | 8.6 | 9.0 | 8.4 | 8.3 |
| Zero Span Breaking Length (km) | 13.1 | 12.7 | 13.3 | 13.4 | 14.1 | 14.2 | 14.6 | 15.2 | 14.4 | 14.3 | 14.0 | 14.0 |
| Air Resistance (Gurley) | 26.3 | 65.7 | 112.5 | 209.0 | 13.3 | 28.8 | 50.1 | 101.7 | 57.4 | 104.0 | 244.3 | 312.5 |
| (sec/100 mL) | | | | | | | | | | | | |
| Sheffield Roughness (mL/min) | 137 | 88 | 70 | 50 | 142 | 103 | 99 | 65 | 98 | 73 | 50 | 40 |
| Brightness | 39 | | | | 38 | | | | 36 | | | |
| Opacity (%) | 97.7 | 96.0 | 95.0 | 93.6 | 96.7 | 94.2 | 92.0 | 91.1 | 94.9 | 92.3 | 89.8 | 89.2 |
| Scattering Coefficient (cm²/g) | 363 | 290 | 252 | 221 | 345 | 264 | 225 | 197 | 268 | 216 | 185 | 169 |
| | 331-1126(1) | | | | 331-1136(1) | | | | 331-1162(3) | | | |
| PFI Revolutions | 0 | 1000 | 3000 | 6000 | 0 | 1000 | 3000 | 6000 | 0 | 1000 | 3000 | 6000 |
| Screened Csf (mL) | 577 | 530 | 476 | 422 | 415 | 409 | 373 | 365 | 497 | 457 | 400 | 346 |
| Apparent Density (kg/m³) | 609 | 695 | 723 | 742 | 620 | 652 | 690 | 678 | 648 | 707 | 751 | 760 |
| Burst Index (kPa · m²/g) | 3.4 | 5.4 | 6.5 | 7.2 | 5.9 | 6.9 | 7.4 | 7.6 | 5.2 | 6.8 | 8.1 | 8.5 |
| Breaking length (km) | 6.7 | 8.0 | 8.9 | 10.1 | 8.8 | 9.3 | 10.0 | 10.6 | 9.5 | 10.3 | 11.2 | 11.5 |
| Tensile Index (N · m/g) | 65.6 | 78.5 | 86.9 | 99.0 | 86.2 | 91.2 | 97.6 | 104.0 | 93.4 | 101.3 | 109.9 | 112.3 |
| Stretch (%) | 1.47 | 2.58 | 3.12 | 3.83 | 3.32 | 3.75 | 4.51 | 5.40 | 2.24 | 3.25 | 3.90 | 4.38 |
| Tear Index (mN · m²/g) (1 Ply) | 6.0 | 8.5 | 8.2 | 8.5 | 7.8 | 8.5 | 8.3 | 8.3 | 8.5 | 7.8 | 8.3 | 8.3 |
| Tear Index (mN · m²/g) (4 Ply) | 8.3 | 9.2 | 9.1 | 8.7 | 8.1 | 8.0 | 7.5 | 7.7 | 9.8 | 9.7 | 9.7 | 9.7 |
| Zero Span Breaking Length (km) | 14.9 | 14.8 | 14.7 | 14.7 | 14.2 | 14.7 | 12.9 | 12.4 | 16.8 | 15.5 | 16.4 | 16.7 |
| Air Resistance (Gurley) | 10.6 | 21.3 | 41.7 | 65.0 | 563.9 | 1128.1 | >30 min | >30 min | 39.0 | 79.3 | 152.3 | 223.8 |
| (sec/100 mL) | | | | | | | | | | | | |
| Sheffield Roughness (mL/min) | 161 | 111 | 92 | 76 | 65 | 32 | 20 | 17 | 100 | 68 | 50 | 41 |
| Brightness | 38 | | | | 33 | | | | 39 | | | |
| Opacity (%) | 96.0 | 94.6 | 92.8 | 92.0 | 95.8 | 95.0 | 93.2 | 91.2 | 96.7 | 95.5 | 94.2 | 93.6 |
| Scattering Coefficient (cm²/g) | 323 | 273 | 238 | 219 | 269 | 233 | 195 | 165 | 344 | 292 | 251 | 234 |
| | 331-1186(3) | | | | 53-242(1) | | | | 53-242(2) | | | |
| PFI Revolutions | 0 | 1000 | 3000 | 6000 | 0 | 1000 | 3000 | 6000 | 0 | 1000 | 3000 | 6000 |
| Screened Csf (mL) | 489 | 481 | 418 | 357 | 569 | 510 | 440 | 389 | 513 | 472 | 405 | 350 |
| Apparent Density (kg/m³) | 673 | 716 | 759 | 770 | 631 | 691 | 723 | 741 | 640 | 722 | 779 | 785 |
| Burst Index (kPa · m²/g) | 6.0 | 7.3 | 8.5 | 8.9 | 4.6 | 6.5 | 7.2 | 7.7 | 5.6 | 7.0 | 8.0 | 8.5 |
| Breaking length (km) | 9.3 | 10.2 | 11.4 | 11.3 | 7.8 | 9.3 | 9.6 | 10.4 | 9.0 | 10.1 | 10.4 | 11.4 |
| Tensile Index (N · m/g) | 91.2 | 100.2 | 112.0 | 110.6 | 76.2 | 91.5 | 94.4 | 102.3 | 88.5 | 98.8 | 102.0 | 111.6 |
| Stretch (%) | 2.25 | 3.55 | 4.65 | 4.59 | 1.76 | 3.39 | 3.68 | 4.15 | 2.21 | 3.18 | 3.65 | 4.49 |
| Tear Index (mN · m²/g) (1 Ply) | 7.5 | 8.6 | 8.7 | 8.4 | 7.5 | 8.3 | 8.7 | 8.5 | 7.7 | 8.7 | 8.8 | 8.4 |
| Tear Index (mN · m²/g) (4 Ply) | 8.5 | 8.7 | 8.2 | 8.5 | 8.4 | 8.6 | 8.6 | 8.7 | 7.8 | 7.7 | 7.5 | 7.6 |
| Zero Span Breaking Length (km) | 15.4 | 15.2 | 15.6 | 15.2 | 16.1 | 16.0 | 16.5 | 15.0 | 14.3 | 13.4 | 15.6 | 14.3 |
| Air Resistance (Gurley) | 79.9 | 166.7 | 294.7 | 538.4 | 32.1 | 80.8 | 148.0 | 271.4 | 72.7 | 136.8 | 243.2 | 402.1 |
| (sec/100 mL) | | | | | | | | | | | | |
| Sheffield Roughness (mL/min) | 74 | 45 | 36 | 26 | 106 | 71 | 54 | 35 | 81 | 55 | 35 | 28 |
| Brightness | 38 | | | | 40 | | | | 39 | | | |
| Opacity (%) | 95.7 | 93.9 | 92.4 | 91.3 | 95.1 | 92.2 | 91.0 | 89.8 | 95.5 | 93.6 | 91.8 | 90.0 |
| Scattering Coefficient (cm²/g) | 302 | 247 | 222 | 194 | 325 | 250 | 224 | 202 | 301 | 249 | 219 | 193 |
| | 53-246(1) | | | | 53-246(2) | | | | 93-968(1) | | | |
| PFI Revolutions | 0 | 1000 | 3000 | 6000 | 0 | 1000 | 3000 | 6000 | 0 | 1000 | 3000 | 6000 |
| Screened Csf (mL) | 549 | 491 | 436 | 385 | 550 | 531 | 468 | 389 | 550 | 508 | 429 | 368 |
| Apparent Density (kg/m³) | 651 | 710 | 746 | 765 | 615 | 707 | 737 | 775 | 617 | 657 | 720 | 737 |
| Burst Index (kPa · m²/g) | 4.3 | 6.5 | 7.3 | 7.7 | 4.3 | 6.1 | 7.2 | 7.3 | 5.0 | 6.4 | 7.3 | 7.6 |
| Breaking length (km) | 8.1 | 9.1 | 10.0 | 10.0 | 7.4 | 8.9 | 9.1 | 10.0 | 9.0 | 8.9 | 10.3 | 10.5 |
| Tensile Index (N · m/g) | 79.7 | 89.2 | 98.3 | 98.5 | 72.6 | 87.3 | 89.0 | 98.5 | 87.8 | 87.6 | 100.9 | 102.6 |
| Stretch (%) | 2.08 | 3.54 | 4.15 | 4.28 | 2.00 | 3.59 | 3.78 | 4.76 | 2.11 | 2.97 | 3.80 | 4.11 |
| Tear Index (mN · m²/g) (1 Ply) | 7.0 | 8.2 | 8.0 | 8.6 | 7.1 | 7.8 | 8.5 | 8.1 | 8.2 | 8.5 | 8.7 | 8.0 |
| Tear Index (mN · m²/g) (4 Ply) | 7.7 | 8.3 | 8.4 | 8.1 | 8.2 | 8.5 | 8.4 | 8.2 | 8.5 | 8.2 | 8.0 | 8.1 |
| Zero Span Breaking Length (km) | 15.5 | 14.5 | 14.7 | 15.4 | 14.9 | 14.6 | 14.0 | 15.3 | 16.2 | 15.0 | 15.6 | 14.9 |
| Air Resistance (Gurley) | 48.2 | 114.9 | 195.2 | 306.4 | 32.8 | 77.0 | 146.0 | 207.4 | 39.0 | 82.2 | 146.1 | 261.2 |
| (sec/100 mL) | | | | | | | | | | | | |
| Sheffield Roughness (mL/min) | 92 | 59 | 40 | 30 | 119 | 75 | 54 | 38 | 113 | 76 | 54 | 43 |
| Brightness | 40 | | | | 40 | | | | 41 | | | |
| Opacity (%) | 95.8 | 93.9 | 92.5 | 90.3 | 96.0 | 94.8 | 91.9 | 91.3 | 95.4 | 93.6 | 92.0 | 91.0 |
| Scattering Coefficient (cm²/g) | 341 | 272 | 235 | 211 | 347 | 287 | 240 | 226 | 333 | 282 | 248 | 228 |

| | 93-968(2) | | | |
|---|---|---|---|---|
| PFI Revolutions | 0 | 1000 | 3000 | 6000 |
| Screened Csf (mL) | 468 | 455 | 409 | 340 |
| Apparent Density (kg/m³) | 555 | 642 | 679 | 690 |
| Burst Index (kPa · m²/g) | 4.5 | 6.0 | 6.9 | 7.5 |
| Breaking length (km) | 8.0 | 9.2 | 10.0 | 10.4 |
| Tensile Index (N · m/g) | 78.7 | 89.9 | 98.0 | 101.9 |
| Stretch (%) | 1.90 | 2.95 | 3.62 | 3.80 |
| Tear Index (mN · m²/g) (1 Ply) | 6.1 | 7.2 | 7.6 | 7.6 |
| Tear Index (mN · m²/g) (4 Ply) | 6.9 | 7.2 | 7.1 | 7.1 |
| Zero Span Breaking Length (km) | 14.2 | 14.7 | 14.6 | 14.4 |
| Air Resistance (Gurley) | 51.3 | 81.1 | 117.7 | 190.4 |
| (sec/100 mL) | | | | |
| Sheffield Roughness (mL/min) | 131 | 91 | 63 | 50 |
| Brightness | 39 | | | |
| Opacity (%) | 95.3 | 94.2 | 93.4 | 92.7 |
| Scattering Coefficient (cm²/g) | 319 | 288 | 263 | 244 |

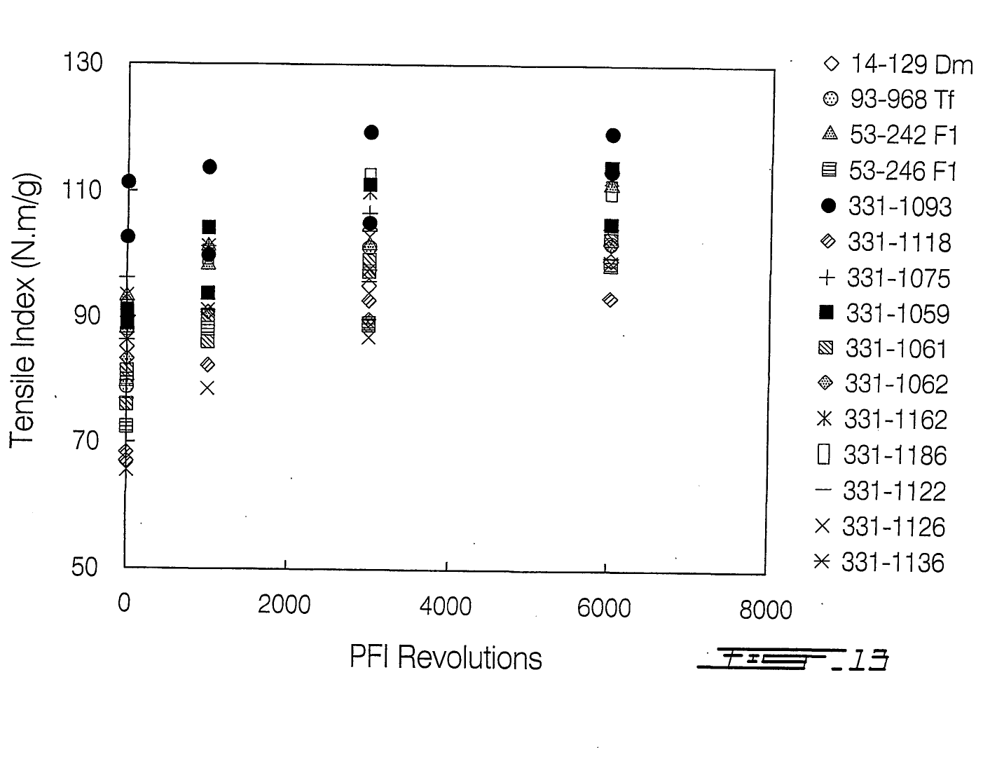

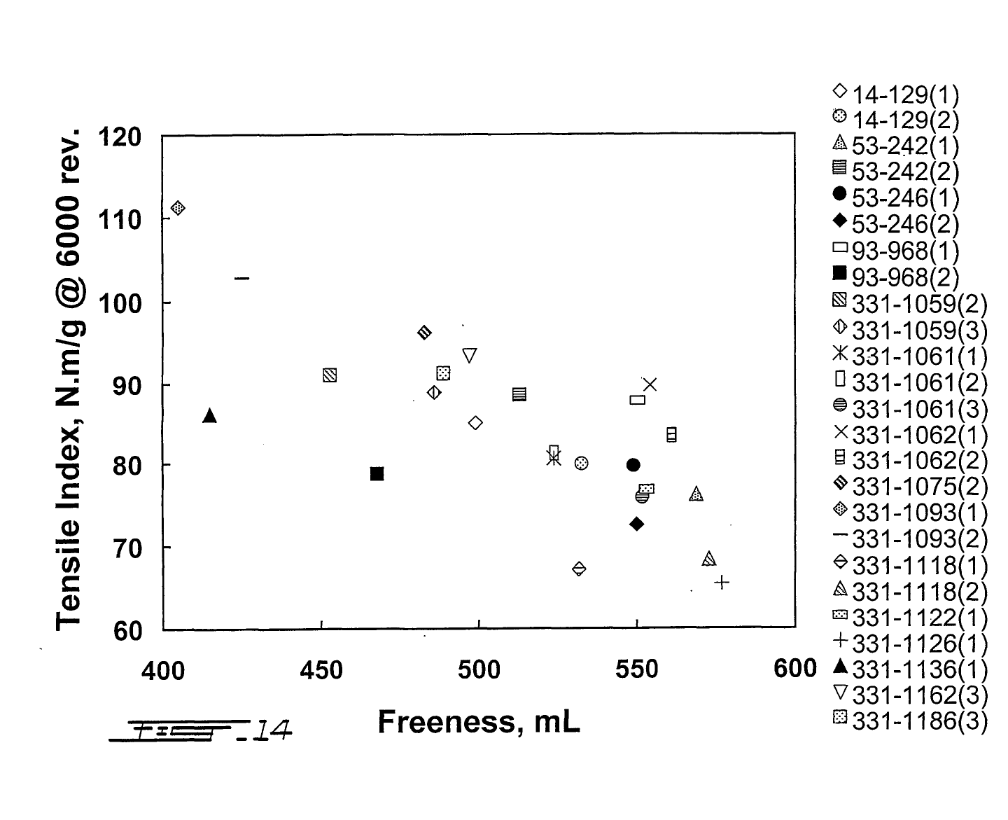

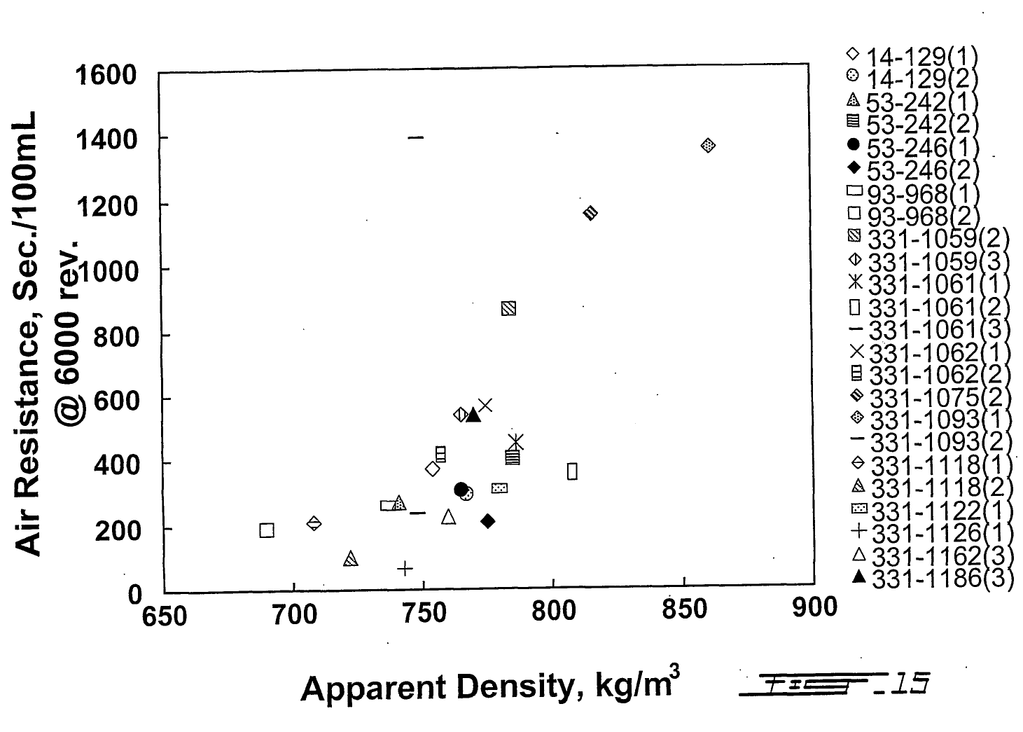

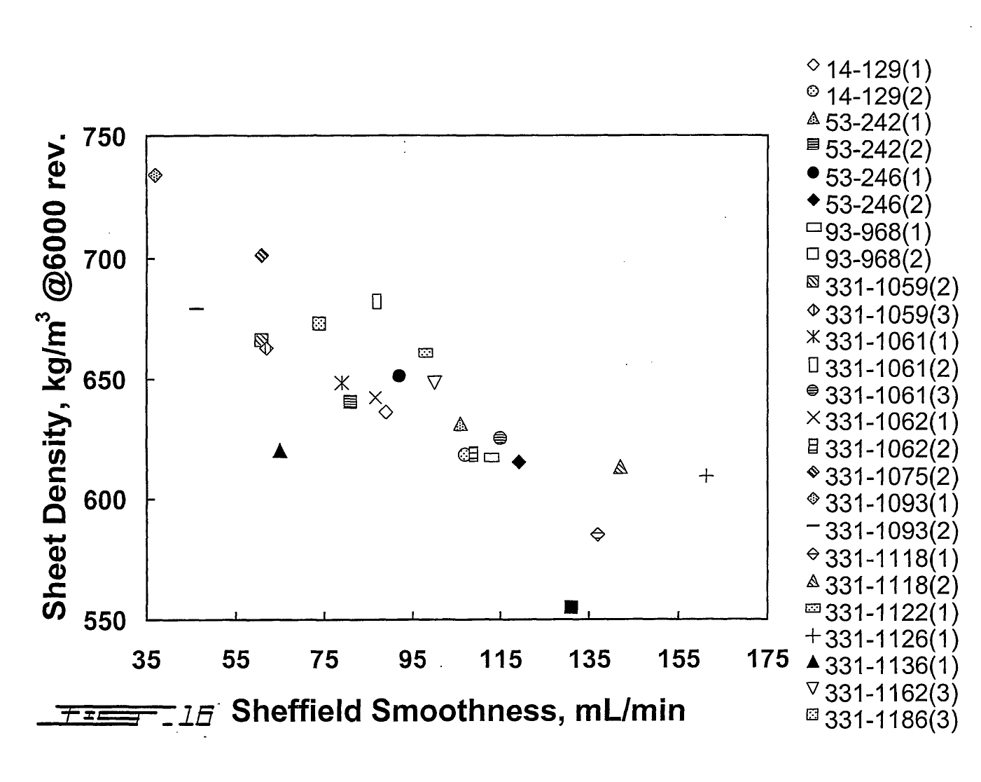

In a plot of tensile index vs. bulk, presented in FIG. 11, it can be seen that there is a strong negative correlation between the properties (Pearson coefficient −0.74, p=0.001). More importantly, some clonal pulps (e.g. 331-1122, 1.26 cm³/g@100 N·m/g) are less bulky at given tensile strengths than are others (e.g. 331-1136, 1.45 cm³/g@100 N·m/g. (FIG. 12)) This was not predicted from the coarseness data in Table XII (331-1122, 0.122 mg/m vs 331-1136, 0.117 mg/m) and highlights the importance of carrying out pilot scale pulping trials. A coarseness cutoff of <0.1 mg/m is adequate for predicting low bulk/high tensile/fine fibres. It is worth noting that for pulps. prepared from eucalyptus species (the major competitor envisaged for Northern Populus plantation resources)—a tensile index value of 70 N·m/g is considered “standard” (Cotterill, P., Macrae, S., and Brolin, A. “Growing eucalyptus for high quality papermaking fibres,” Appita J. 52(2), 79 (1999)). Most of the hybrid poplar pulps examined in this study exceed that strength value even in an unbeaten state (FIG. 13). Additionally, the wide range of tensile indices suggest that there is wide variation in cell wall properties amongst the clones, a possibility which opens up potential multiple end-use applications for the pulps.

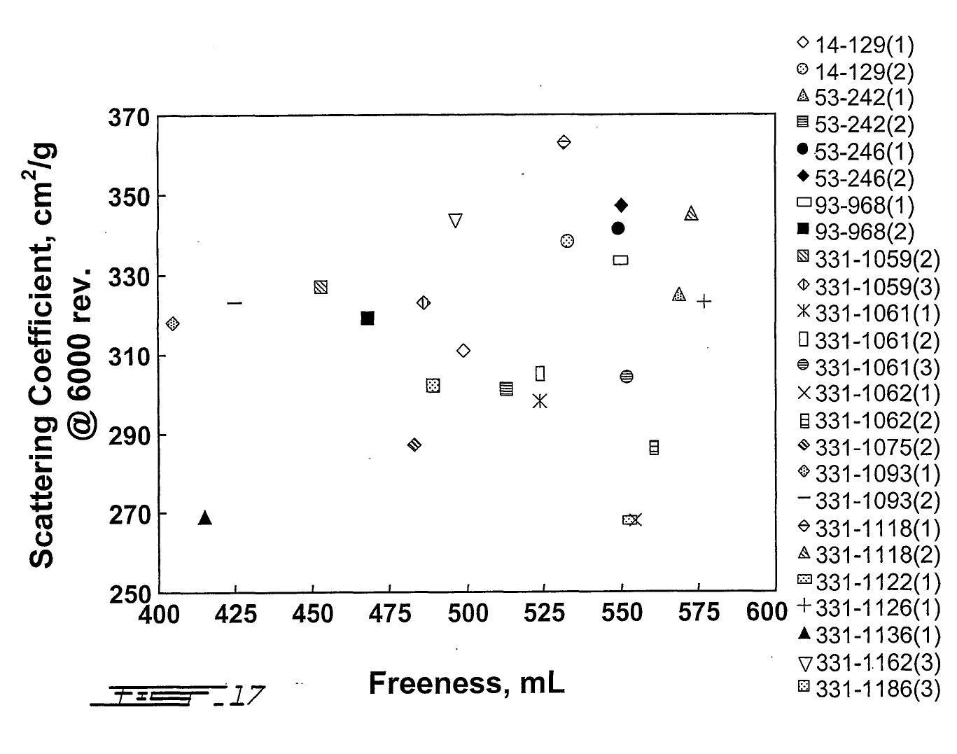

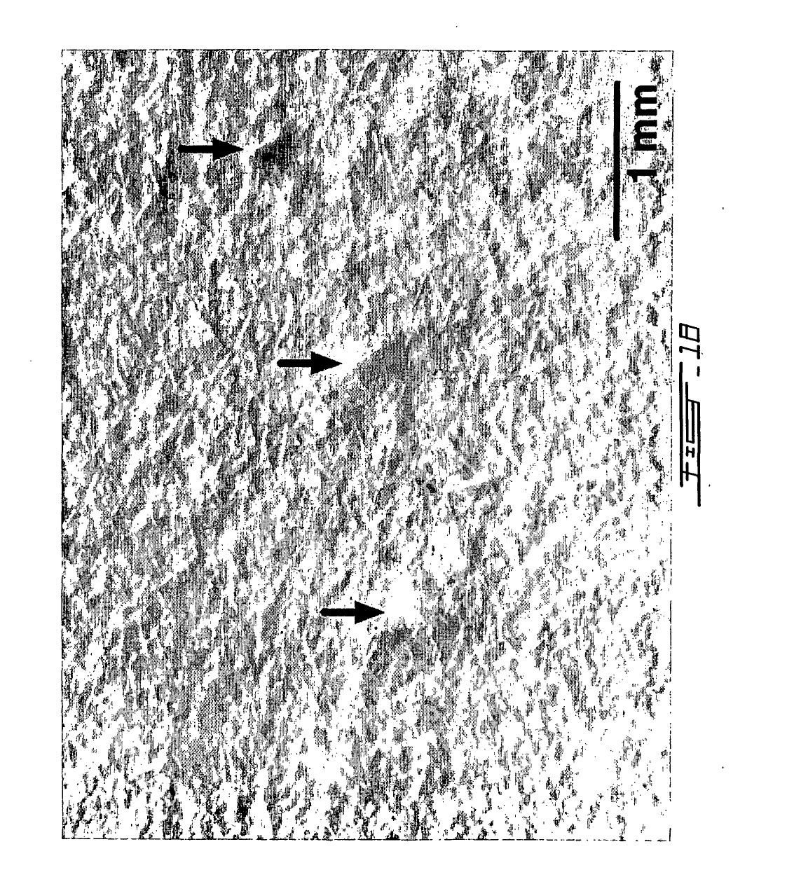

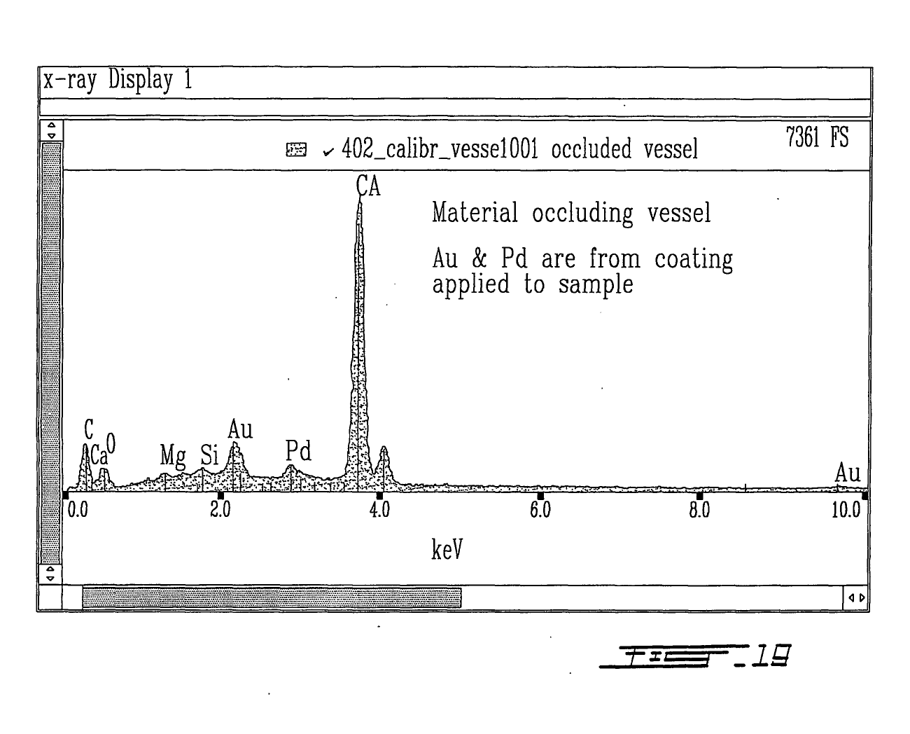

The wide range of cell sizes is further confirmed by the range of tensile indices observed at a given freeness, (a strongly negative relationship between tensile index and freeness properties exists Pearson coefficient −0.74, p=0.001; FIG. 14). Similarly the relationship of air resistance (Gurley) to sheet density, presented in FIG. 15, shows the wide ranging results consequent from cell wall property differences. For example, at beating levels of 6000 PFI revolutions, clones 331-1093 and 331-1075 exhibit the high tensile indices (116.1 and 113.1 N·m/g respectively) coupled with high air resistances (1364.7 and 1152.7 sec/100 mL respectively) which indicate that they possess thinner cell walls than do the other clonal pulps. By contrast, the pulp from clone 53-246 possesses the low tensile index and low air resistance values typical of a thicker cell-walled fibre (98.5 N·m/g, 256.9 sec/100 mL). Interestingly, the high calcium-containing pulp obtained from clone 331-1136 forms an outlier point for this analysis, exhibiting a combination of lower tensile strength (104.0 N·m/g) and very high air resistance (>30 min/100 mL). These variations mirror that seen in a separate study on a population of natural aspen clones. Again the potential for producing pulps for different end-use applications is clear and should be emphasized.

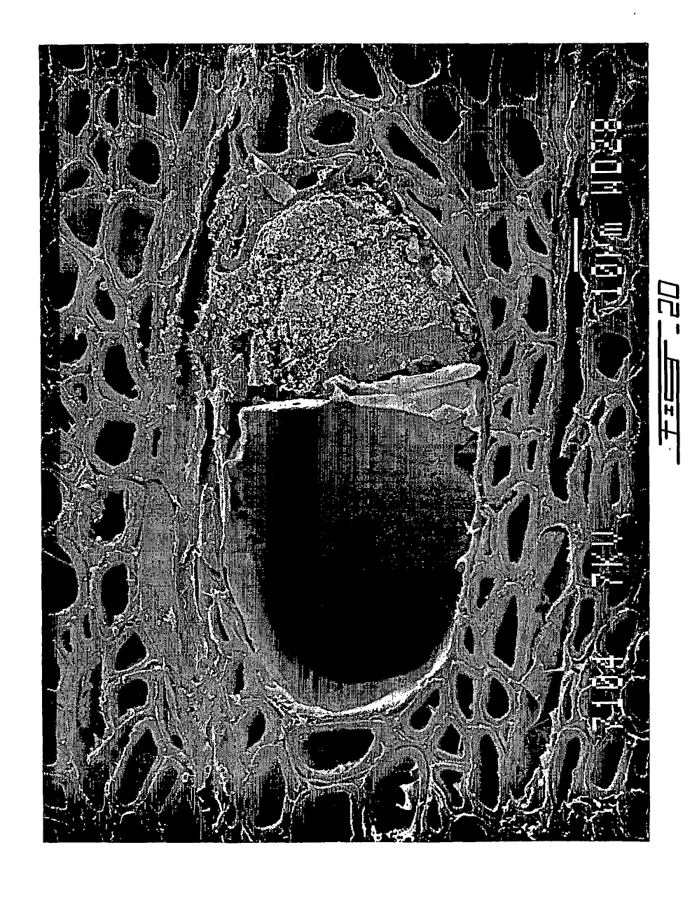

A number of the kraft pulping properties described here were used in a QTL mapping experiment to attempt to determine the chromosomal locations of any genes involved in the control of these important properties. The outcomes of this analysis are presented in the QTL mapping results section. In terms of sheet formation properties, smoothness shows significant relationships with freeness (Pearson coefficient 0.76, p=0.000) tensile strength (Pearson coefficient −0.87, p=0.000), and sheet density (Pearson coefficient −0.81, p=0.000; FIG. 16).

Optical Properties

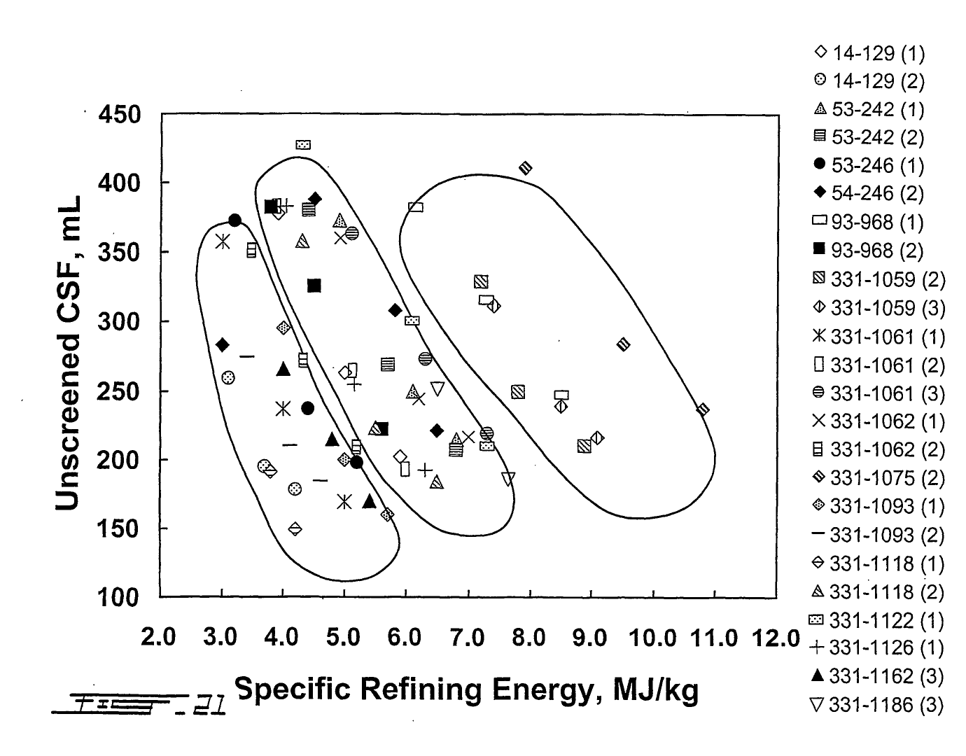

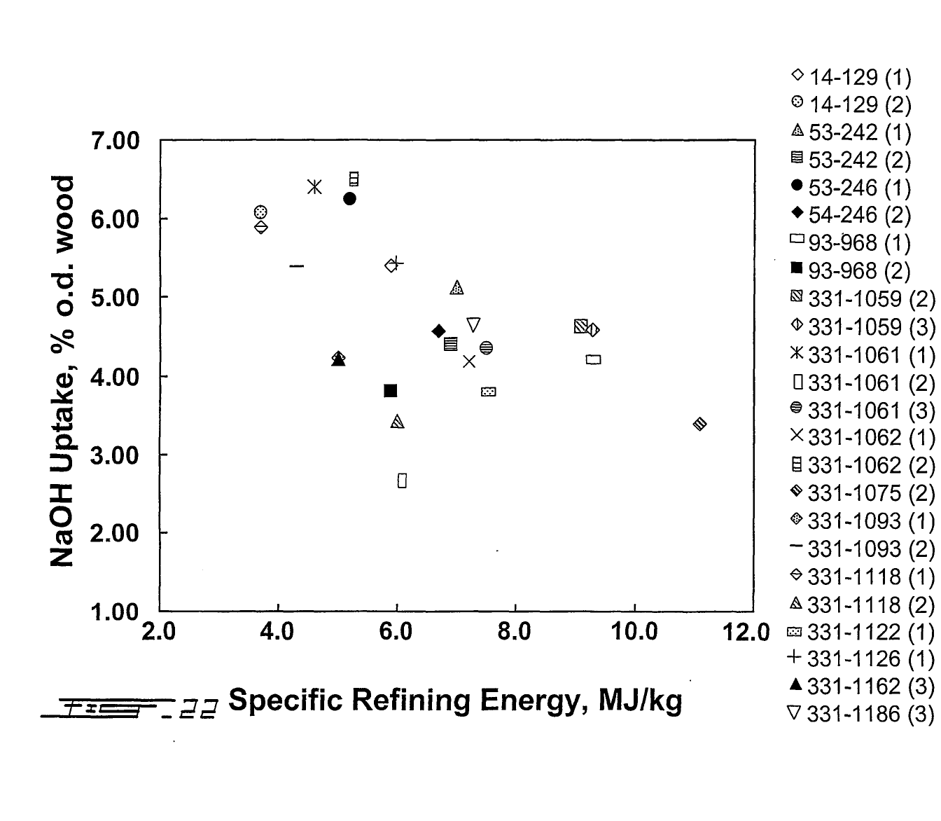

Hardwood kraft pulps principally impart optical and surface properties to paper rather than simply strength parameters. FIG. 17 shows the wide range of pulp-scattering coefficients obtained from the unbleached clonal pulps at various freeness levels (at 0 PFI rev., the range is 268-363 cm²/g). A number of the pulps are exceptional (e.g. 331-1118)—even compared to aspen clones. For the purposes of comparison with the major competitive species, it should be noted that typical eucalypt pulps (Eucalyptus nitens samples) give scattering coefficients over a very similar range, 286-360 cm²/g [Kibblewhite, R. P., Riddell, M. J. C. and Shelbourne, C. J. A. Kraft fibre and pulp qualities of 29 trees of New Zealand grown Eucalyptus nitens. APPITA J. 51(2), 114-121 (1998)].

Handsheet Analyses—Calcium Accumulation

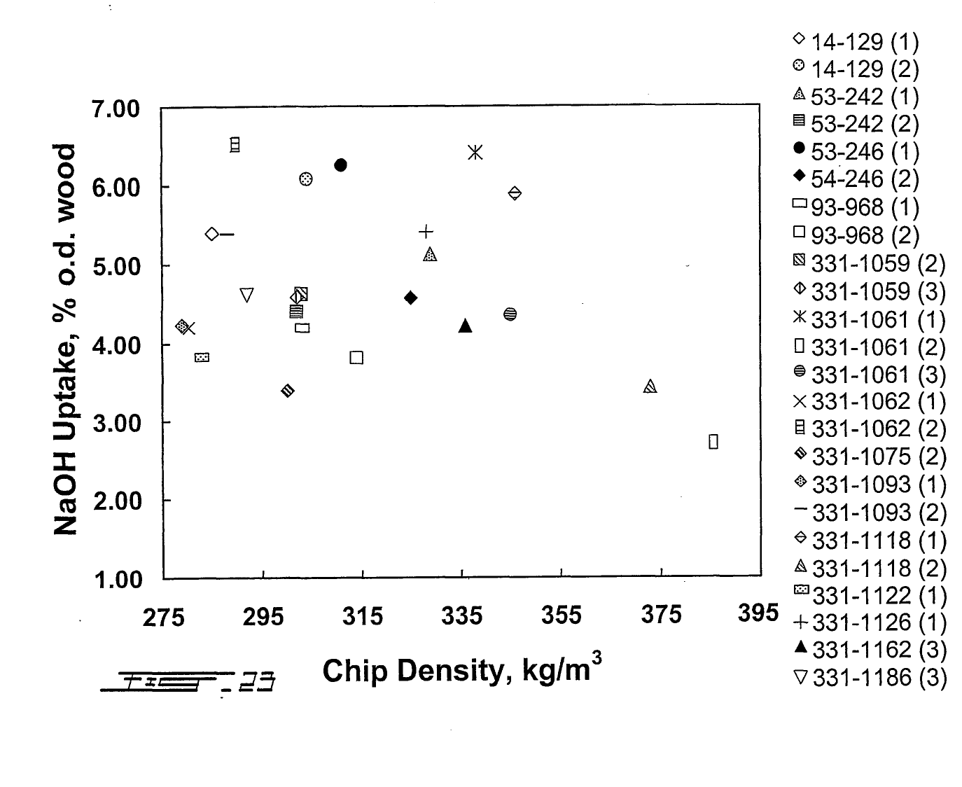

It was readily evident from a visual inspection of the resultant sheets that some unusual surface deformations, in the form of raised “bumps” approximately 1 mm in diameter, were prevalent (FIG. 18). The deformations were present in handsheets made after various levels of beating using standard PFI protocols (0-6000 rev.). It could also be observed that these deformations were present to a greater or lesser degree in the sheets dependent on the clonal source of the corresponding pulps. Sheets from the pulps were rated for the numbers of deformations using an arbitrary scale for visual inspection (similar to the ranking system used for assessing pest damage to hybrid poplars in pest-resistance QTL mapping studies (Patterson, A. H., Lander, E. S., Hewitt, J. D., Peterson, S., and Lincoln, S. E. “Resolution of quantitative traits into Mendelian factors by using a complete linkage map of restriction fragment linked polymorphisms,” Nature 335, 721-726 (1988)). The ratings for each genotype analyzed are tabulated in Table XV.

TABLE XV
Arbitrary scale rating of degree of surface
deformation accumulation in test handsheets

| Genotype | Handsheet Deformation Rating | Number of Clones |
|---|---|---|
| ILL-129 | 1.5 | 2 |
| 93-968 | 3 | 2 |
| 53-246 | 2 | 2 |
| 53-242 | 3 | 2 |
| 331-1059 | 2.5 | 2 |
| 331-1061 | 2 | 3 |
| 331-1062 | 2.5 | 2 |
| 331-1075 | 0 | 1 |
| 331-1093 | 3 | 2 |
| 331-1118 | 3.5 | 2 |
| 331-1122 | 2 | 1 |
| 331-1126 | 0 | 1 |
| 331-1136 | 4 | 1 |
| 331-1162 | 3 | 1 |
| 331-1186 | 3 | 1 |

The results of the MAPMAKER-QTL 1.1 analysis performed using the phenotypic ranking data obtained from handsheet analyses (Table XV) of each of the poplar clones are presented in Table XVI below.

TABLE XVI
Significant QTL detected for calcium deposition

| | Trait | | | | | |
|---|---|---|---|---|---|---|
| | | LOD | | Length/ | | |
| | Marker/Linkage | Score | Phen % | cM | Weight | Dom. |
| Calcium | P1150-H07_10/N | 2.94 | 81.7 | 13.8 | 0.3286 | −1.7214 |
| deposits | | | | | | |

Microscopy and X-ray Analysis of Crystalline Deposits

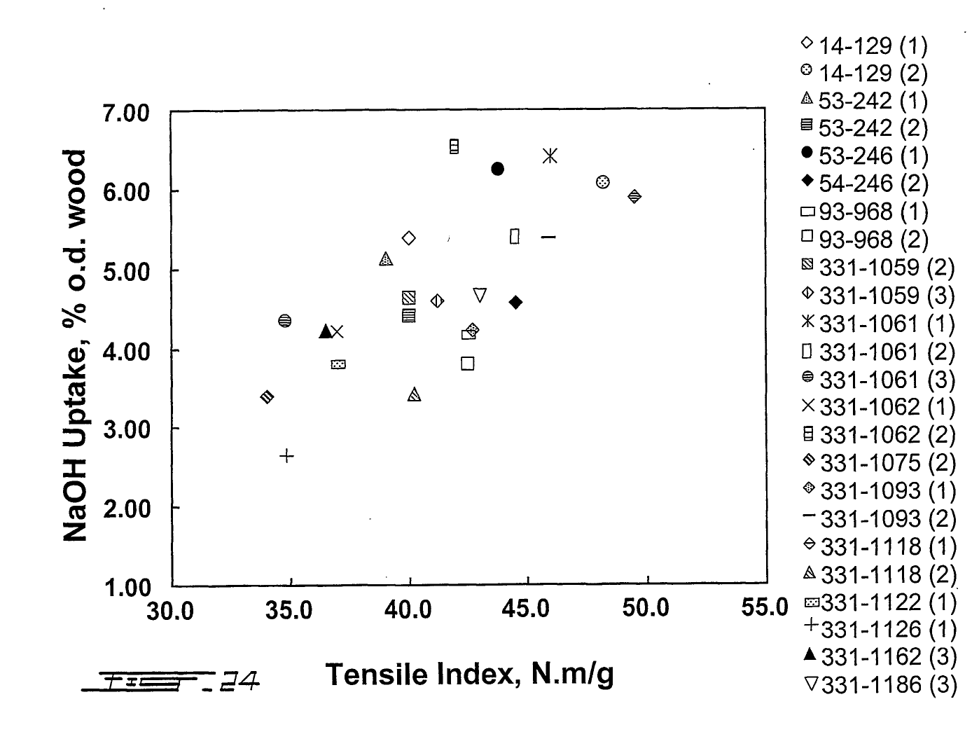

On further investigation, the deformations were found to be caused by a crystalline deposit found in some vessel elements' in the pulp samples used to make the handsheets. These deposits were characterized by SEM/EDS and were found to consist primarily of calcium salts (FIG. 19) (Reath, S., Hussein, A., Gee, W., Lawrence, V., Drummond, J., and Potter, S. “Calcium accumulation in the wood of short rotation cottonwood species: effects on pulp properties,” Wood Sci. Tech. (submitted, 2001)). This agrees well with previous literature observations of the same phenomenon (Janin, G. and Clement, A. “Calcium carbonate crystals in the wood of poplars. Effect on the distribution of mineral ions related to the formation of heartwood,” Ann. Sci. For. 29, 67-105 (1972)).

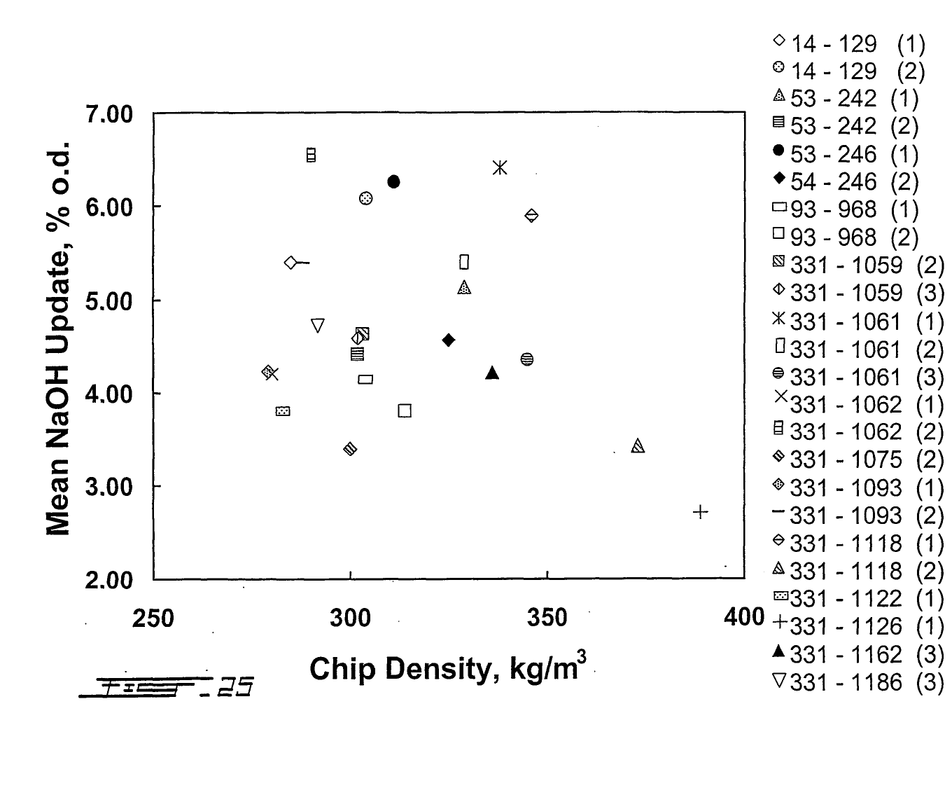

Examination of wood chips taken from the poplar clones by light microscopy and SEM also revealed the calcium deposits and, more intriguingly, their specific and exclusive nature. FIG. 20 shows an electron micrograph of two adjacent vessel elements in a wood chip, one of which is completely occluded with a deposit. By contrast, the adjacent element is completely free of crystals. Contrary to some literature reports [Muhammad, A. F. and Micko, M. M. “Accumulation of calcium crystals in the decayed wood of aspen attacked by Fomes igniarius,” IAWA B. 5, 237-241 (1984)], the deposits seen in this study (as examined microscopically) do not appear to be associated with any form of fungal attack or other decay process.

Alkaline Peroxide Refiner Mechanical Pulping

The raw data for the Alkaline Peroxide Refiner Mechanical Pulping (APRMP) from each of 15 hybrid poplar clones consisting of 24 hybrid poplar trees are shown in Table XVII. In general, appropriate baseline values of pulp freeness and specific refining energy are the two parameters commonly used to monitor mechanical and optical properties of APRMP pulps. Thus, to facilitate data analysis and discussion , the raw data were standardized by interpolation or extrapolation to a freeness of 200 mL CSF (Table XVIII) and a specific refining energy (SRE) of 6.0 MJ/kg (Table XIX).

TABLE XVIII
Properties of APRMP Pulps from Hybrid Poplars

| | 14-129 (1) | | | 14-129 (2) | | |
|---|---|---|---|---|---|---|
| | 1466-4 | 1466-3 | 1466-2 | 1473-4 | 1473-3 | 1473-2 |
| Unscreened CSF (mL) | 202 | 263 | 378 | 178 | 195 | 259 |
| Specific Energy (MJ/kg) | 5.9 | 5.0 | 3.9 | 4.2 | 3.7 | 3.1 |
| Screened CSF (mL) | 208 | 274 | 408 | 181 | 206 | 266 |
| Reject (% o.d. pulp) | 0.0 | 0.0 | 0.1 | 0.0 | 0.0 | 0.1 |
| Apparent Sheet Density (kg/m³) | 388 | 380 | 350 | 464 | 458 | 439 |
| Burst Index (Kpa · m²/g) | 2.0 | 1.8 | 1.5 | 2.7 | 2.6 | 2.5 |
| Breaking length (km) | 4.0 | 3.8 | 2.9 | 5.1 | 4.8 | 4.4 |
| Tensile Index (N · m/g) | 39.1 | 36.8 | 28.4 | 50.1 | 47.5 | 42.8 |
| Stretch (%) | 1.57 | 1.49 | 1.16 | 1.97 | 1.83 | 1.66 |
| Tear Index (mN · m²/g) (4-Ply) | 5.5 | 5.7 | 5.1 | 6.1 | 6.3 | 6.3 |
| Sheffield Roughness (SU) | 137 | 167 | 268 | 105 | 115 | 123 |
| Brightness (%) | 78 | 79 | 79 | 77 | 78 | 78 |
| Opacity (%) | 85.5 | 85.0 | 84.5 | 82.4 | 81.4 | 81.6 |
| Scattering Coefficient (cm²/g) | 510 | 506 | 503 | 416 | 416 | 418 |
| R - 48 fraction (%) | 43.6 | 46.1 | 50.0 | 43.4 | 43.2 | 44.6 |
| Fines (P-200) (%) | 14.1 | 13.1 | 12.0 | 14.1 | 13.9 | 14.2 |
| W. Weighted Average Fibre Length (mm) | 1.00 | 1.06 | 1.20 | 0.99 | 0.97 | 1.03 |
| L. Weighted Average Fibre Length (mm) | 0.78 | 0.80 | 0.84 | 0.78 | 0.78 | 0.79 |
| Arithmetic Average Fibre Length (mm) | 0.54 | 0.54 | 0.54 | 0.54 | 0.54 | 0.54 |
| | 53-242 (1) | | | 53-242 (2) | | |
| | 1458-4 | 1458-3 | 1458-2 | 1452-4 | 1452-3 | 1452-2 |
| Unscreened CSF (mL) | 215 | 250 | 373 | 207 | 269 | 380 |
| Specific Energy (MJ/kg) | 6.8 | 6.1 | 4.9 | 6.8 | 5.7 | 4.4 |
| Screened CSF (mL) | 211 | 275 | 372 | 220 | 262 | 378 |
| Reject (% o.d. pulp) | 0.0 | 0.0 | 0.2 | 0.0 | 0.0 | 0.2 |
| Apparent Sheet Density (kg/m³) | 390 | 377 | 359 | 395 | 386 | 364 |
| Burst Index (Kpa · m²/g) | 2.1 | 2.0 | 1.8 | 2.1 | 2.0 | 1.7 |
| Breaking length (km) | 3.9 | 3.5 | 3.3 | 4.0 | 3.7 | 3.5 |
| Tensile Index (N · m/g) | 38.5 | 34.7 | 32.5 | 39.2 | 36.3 | 34.1 |
| Stretch (%) | 1.67 | 1.40 | 1.44 | 1.52 | 1.38 | 1.41 |
| Tear Index (mN · m²/g) (4-Ply) | 5.7 | 5.8 | 6.1 | 5.3 | 5.4 | 5.5 |
| Sheffield Roughness (SU) | 133 | 156 | 227 | 126 | 158 | 237 |
| Brightness (%) | 75 | 76 | 76 | 75 | 76 | 76 |
| Opacity (%) | 86.5 | 86.0 | 85.2 | 86.9 | 85.8 | 85.2 |
| Scattering Coefficient (cm²/g) | 498 | 498 | 489 | 500 | 492 | 482 |
| R - 48 fraction (%) | 49.1 | 49.2 | 54.1 | 45.4 | 47.2 | 52.5 |
| Fines (P-200) (%) | 16.9 | 17.2 | 14.1 | 14.8 | 14.4 | 12.5 |
| W. Weighted Average Fibre Length (mm) | 1.06 | 1.08 | 1.11 | 0.97 | 1.00 | 1.12 |
| L. Weighted Average Fibre Length (mm) | 0.84 | 0.84 | 0.86 | 0.77 | 0.78 | 0.81 |
| Arithmetic Average Fibre Length (mm) | 0.57 | 0.56 | 0.57 | 0.52 | 0.53 | 0.54 |
| | 53-246 (1) | | | 53-246 (2) | | |
| | 1472-4 | 1472-3 | 1472-2 | 1460-4 | 1461-3 | 1461-2 |
| Unscreened CSF (mL) | 198 | 237 | 372 | 221 | 308 | 388 |
| Specific Energy (MJ/kg) | 5.2 | 4.4 | 3.2 | 6.5 | 5.8 | 4.5 |
| Screened CSF (mL) | 184 | 236 | 374 | 227 | 326 | 416 |
| Reject (% o.d. pulp) | 0.1 | 0.1 | 0.7 | 0.0 | 0.1 | 0.5 |
| Apparent Sheet Density (kg/m³) | 425 | 403 | 382 | 440 | 401 | 374 |
| Burst Index (Kpa · m²/g) | 2.6 | 2.4 | 2.0 | 2.3 | 2.1 | 1.8 |
| Breaking length (km) | 4.6 | 4.3 | 3.8 | 4.4 | 3.8 | 3.3 |
| Tensile Index (N · m/g) | 44.7 | 42.1 | 37.1 | 42.8 | 37.6 | 32.1 |
| Stretch (%) | 1.89 | 1.64 | 1.51 | 1.99 | 1.69 | 1.37 |
| Tear Index (mN · m²/g) (4-Ply) | 6.8 | 6.5 | 6.5 | 6.2 | 6.3 | 6.4 |
| Sheffield Roughness (SU) | 117 | 122 | 213 | 110 | 152 | 231 |
| Brightness (%) | 79 | 79 | 79 | 76 | 76 | 77 |
| Opacity (%) | 82.5 | 81.7 | 81.5 | 86.8 | 85.8 | 85.1 |
| Scattering Coefficient (cm²/g) | 435 | 428 | 427 | 501 | 488 | 473 |
| R - 48 fraction (%) | 46.5 | 48.8 | 52.5 | 47.4 | 50.2 | 55.4 |
| Fines (P-200) (%) | 15.0 | 13.4 | 12.2 | 14.9 | 15.4 | 11.3 |
| W. Weighted Average Fibre Length (mm) | 1.05 | 1.11 | 1.16 | 1.02 | 1.15 | 1.19 |
| L. Weighted Average Fibre Length (mm) | 0.81 | 0.83 | 0.86 | 0.82 | 0.87 | 0.89 |
| Arithmetic Average Fibre Length (mm) | 0.54 | 0.55 | 0.55 | 0.55 | 0.56 | 0.56 |
| | 93-968 (1) | | | 93-968 (2) | | |
| | 1459-5 | 1459-4 | 1459-3 | 1450-3 | 1450-2 | 1451-2 |
| Unscreened CSF (mL) | 246 | 315 | 382 | 222 | 325 | 382 |
| Specific Energy (MJ/kg) | 8.5 | 7.3 | 6.1 | 5.6 | 4.5 | 3.8 |
| Screened CSF (mL) | 256 | 304 | 377 | 236 | 344 | 398 |
| Reject (% o.d. pulp) | 0.0 | 0.1 | 0.1 | 0.0 | 0.1 | 0.9 |
| Apparent Sheet Density (kg/m³) | 399 | 368 | 361 | 405 | 379 | 357 |
| Burst Index (Kpa · m²/g) | 2.2 | 1.9 | 1.8 | 2.2 | 1.9 | 1.8 |
| Breaking length (km) | 4.1 | 3.7 | 3.4 | 4.2 | 3.5 | 3.5 |
| Tensile Index (N · m/g) | 39.8 | 36.3 | 33.1 | 41.2 | 34.6 | 34.3 |
| Stretch (%) | 1.82 | 1.54 | 1.40 | 1.51 | 1.33 | 1.28 |
| Tear Index (mN · m²/g) (4-Ply) | 6.1 | 5.9 | 6.2 | 5.9 | 5.7 | 5.7 |
| Sheffield Roughness (SU) | 127 | 169 | 216 | 124 | 194 | 245 |
| Brightness (%) | 75 | 75 | 76 | 74 | 75 | 75 |
| Opacity (%) | 89.1 | 88.6 | 87.1 | 88.1 | 87.1 | 85.9 |
| Scattering Coefficient (cm²/g) | 534 | 528 | 510 | 522 | 516 | 487 |
| R - 48 fraction (%) | 43.6 | 51.3 | 56.5 | 45.4 | 50.9 | 54.5 |
| Fines (P-200) (%) | 15.5 | 13.5 | 12.6 | 15.5 | 14.0 | 12.5 |
| W. Weighted Average Fibre Length (mm) | 1.09 | 1.15 | 1.22 | 1.05 | 1.09 | 1.28 |
| L. Weighted Average Fibre Length (mm) | 0.87 | 0.89 | 0.92 | 0.81 | 0.83 | 0.90 |
| Arithmetic Average Fibre Length (mm) | 0.61 | 0.60 | 0.61 | 0.56 | 0.56 | 0.58 |
| | 331-1059 (2) | | | 331-1059 (3) | | |
| | 1453-3 | 1457-3 | 1453-2 | 1454-3 | 1455-3 | 1455-2 |
| Unscreened CSF (mL) | 210 | 249 | 329 | 216 | 239 | 312 |
| Specific Energy (MJ/kg) | 8.9 | 7.8 | 7.2 | 9.1 | 8.5 | 7.4 |
| Screened CSF (mL) | 230 | 257 | 336 | 212 | 250 | 314 |
| Reject (% o.d. pulp) | 0.1 | 0.6 | 0.8 | 0.3 | 0.8 | 1.9 |
| Apparent Sheet Density (kg/m³) | 378 | 363 | 352 | 376 | 350 | 350 |
| Burst Index (Kpa · m²/g) | 2.2 | 2.2 | 1.9 | 2.3 | 2.2 | 2.0 |
| Breaking length (km) | 3.9 | 3.8 | 3.5 | 4.2 | 4.0 | 3.7 |
| Tensile Index (N · m/g) | 38.5 | 37.6 | 33.9 | 40.9 | 38.7 | 36.3 |
| Stretch (%) | 1.84 | 1.70 | 1.58 | 2.01 | 1.89 | 1.65 |
| Tear Index (mN · m²/g) (4-Ply) | 5.1 | 6.3 | 5.7 | 6.2 | 6.3 | 6.2 |
| Sheffield Roughness (SU) | 138 | 151 | 181 | 143 | 157 | 187 |
| Brightness (%) | 75 | 75 | 76 | 78 | 78 | 78 |
| Opacity (%) | 88.7 | 87.4 | 87.1 | 87.4 | 86.5 | 86.5 |
| Scattering Coefficient (cm²/g) | 559 | 518 | 528 | 548 | 537 | 530 |
| R - 48 fraction (%) | 46.8 | 51.2 | 51.0 | 49.2 | 50.4 | 53.6 |
| Fines (P-200) (%) | 17.1 | 15.9 | 16.2 | 16.6 | 17.6 | 14.0 |
| W. Weighted Average Fibre Length (mm) | 1.03 | 1.18 | 1.14 | 1.07 | 1.16 | 1.20 |
| L. Weighted Average Fibre Length (mm) | 0.78 | 0.82 | 0.81 | 0.79 | 0.81 | 0.83 |
| Arithmetic Average Fibre Length (mm) | 0.51 | 0.51 | 0.52 | 0.52 | 0.52 | 0.52 |
| | 331-1061 (1) | | | 331-1061 (2) | | |
| | 1476-4 | 1476-3 | 1476-2 | 1474-4 | 1474-3 | 1474-2 |
| Unscreened CSF (mL) | 169 | 237 | 357 | 194 | 265 | 383 |
| Specific Energy (MJ/kg) | 5.0 | 4.0 | 3.0 | 6.0 | 5.1 | 3.9 |
| Screened CSF (mL) | 190 | 248 | 380 | 205 | 264 | 375 |
| Reject (% o.d. pulp) | 0.0 | 0.1 | 0.3 | 0.0 | 0.1 | 0.3 |
| Apparent Sheet Density (kg/m³) | 426 | 399 | 390 | 386 | 381 | 356 |
| Burst Index (Kpa · m²/g) | 2.7 | 2.4 | 2.1 | 2.2 | 2.2 | 1.9 |
| Breaking length (km) | 4.9 | 4.2 | 3.7 | 4.5 | 3.9 | 3.5 |
| Tensile Index (N · m/g) | 48.2. | 41.0 | 36.6 | 44.2 | 38.4 | 34.2 |
| Stretch (%) | 1.81 | 1.39 | 1.40 | 1.83 | 1.40 | 1.32 |
| Tear Index (mN · m²/g) (4-Ply) | 6.2 | 5.6 | 6.1 | 5.6 | 5.7 | 5.7 |
| Sheffield Roughness (SU) | 99 | 130 | 219 | 130 | 156 | 239 |
| Brightness (%) | 76 | 77 | 78 | 76 | 77 | 78 |
| Opacity (%) | 80.5 | 80.5 | 79.8 | 85.7 | 84.2 | 83.8 |
| Scattering Coefficient (cm²/g) | 387 | 394 | 391 | 482 | 471 | 465 |
| R - 48 fraction (%) | 48.1 | 50.2 | 53.8 | 46.9 | 49.3 | 56.0 |
| Fines (P-200) (%) | 15.1 | 14.9 | 9.8 | 14.5 | 11.9 | 12.7 |
| W. Weighted Average Fibre Length (mm) | 1.07 | 1.11 | 1.19 | 1.06 | 1.08 | 1.21 |
| L. Weighted Average Fibre Length (mm) | 0.83 | 0.86 | 0.89 | 0.78 | 0.79 | 0.84 |
| Arithmetic Average Fibre Length (mm) | 0.54 | 0.56 | 0.57 | 0.52 | 0.53 | 0.53 |
| | 331-1061 (3) | | | 331-1062 (1) | | |
| | 1475-5 | 1475-4 | 1475-3 | 1456-4 | 1456-3 | 1456-2 |
| Unscreened CSF (mL) | 219 | 273 | 363 | 220 | 247 | 361 |
| Specific Energy (MJ/kg) | 7.3 | 6.3 | 5.1 | 7.0 | 6.2 | 4.9 |
| Screened CSF (mL) | 226 | 301 | 371 | 231 | 270 | 359 |
| Reject (% o.d. pulp) | 0.0 | 0.1 | 0.1 | 0.0 | 0.1 | 0.5 |
| Apparent Sheet Density (kg/m³) | 359 | 354 | 336 | 374 | 370 | 349 |
| Burst Index (Kpa · m²/g) | 1.9 | 1.7 | 1.6 | 1.9 | 1.9 | 1.6 |
| Breaking length (km) | 3.4 | 3.3 | 2.9 | 3.6 | 3.4 | 3.2 |
| Tensile Index (N · m/g) | 33.5 | 31.9 | 28.2 | 35.5 | 33.2 | 31.4 |
| Stretch (%) | 1.25 | 1.35 | 1.15 | 1.43 | 1.31 | 1.34 |
| Tear Index (mN · m²/g) (4-Ply) | 5.0 | 5.0 | 4.9 | 5.5 | 5.6 | 5.7 |
| Sheffield Roughness (SU) | 168 | 219 | 276 | 132 | 156 | 225 |
| Brightness (%) | 78 | 79 | 80 | 77 | 77 | 77 |
| Opacity (%) | 84.9 | 83.7 | 83.0 | 86.2 | 85.8 | 84.7 |
| Scattering Coefficient (cm²/g) | 490 | 478 | 466 | 498 | 501 | 482 |
| R - 48 fraction (%) | 48.0 | 54.0 | 56.1 | 51.6 | 53.7 | 57.2 |
| Fines (P-200) (%) | 15.4 | 13.6 | 11.4 | 17.4 | 17.0 | 13.5 |
| W. Weighted Average Fibre Length (mm) | 1.04 | 1.06 | 1.18 | 1.13 | 1.22 | 1.30 |
| L. Weighted Average Fibre Length (mm) | 0.81 | 0.81 | 0.85 | 0.87 | 0.89 | 0.92 |
| Arithmetic Average Fibre Length (mm) | 0.52 | 0.53 | 0.53 | 0.55 | 0.55 | 0.56 |
| | 331-1062 (2) | | | 331-1075 (2) | | |
| | 1462-4 | 1462-3 | 1462-2 | 1444-4 | 1444-3 | 1446 |
| Unscreened CSF (mL) | 209 | 273 | 351 | 237 | 284 | 411 |
| Specific Energy (MJ/kg) | 5.2 | 4.3 | 3.5 | 10.8 | 9.5 | 7.9 |
| Screened CSF (mL) | 225 | 289 | 359 | 250 | 297 | 422 |
| Reject (% o.d. pulp) | 0.0 | 0.0 | 0.1 | 0.1 | 0.1 | 0.3 |
| Apparent Sheet Density (kg/m³) | 409 | 397 | 386 | 344 | 324 | 309 |
| Burst Index (Kpa · m²/g) | 2.1 | 2.1 | 1.9 | 1.7 | 1.6 | 1.3 |
| Breaking length (km) | 4.1 | 4.1 | 3.7 | 3.3 | 2.8 | 2.5 |
| Tensile Index (N · m/g) | 40.6 | 40.3 | 36.3 | 32.0 | 27.4 | 24.8 |
| Stretch (%) | 1.39 | 1.46 | 1.29 | 1.36 | 1.21 | 1.23 |
| Tear Index (mN · m²/g) (4-Ply) | 5.4 | 5.4. | 5.4 | 4.8 | 4.7 | 4.3 |
| Sheffield Roughness (SU) | 116 | 135 | 208 | 182 | 235 | 306 |
| Brightness (%) | 77 | 77 | 78 | 75 | 75 | 76 |
| Opacity (%) | 85.4 | 84.0 | 83.4 | 89.3 | 88.6 | 88.1 |
| Scattering Coefficient (cm²/g) | 492 | 460 | 458 | 577 | 556 | 549 |
| R - 48 fraction (%) | 47.2 | 48.6 | 52.9 | 41.0 | 46.4 | 50.0 |
| Fines (P-200) (%) | 15.7 | 15.8 | 13.1 | 18.6 | 17.3 | 14.2 |
| W. Weighted Average Fibre Length (mm) | 1.07 | 1.15 | 1.10 | 0.99 | 1.07 | 1.15 |
| L. Weighted Average Fibre Length (mm) | 0.83 | 0.87 | 0.85 | 0.78 | 0.80 | 0.83 |
| Arithmetic Average Fibre Length (mm) | 0.56 | 0.58 | 0.56 | 0.54 | 0.54 | 0.54 |
| | 331-1093 (1) | | | 331-1093 (2) | | |
| | 1470-4 | 1470-3 | 1470-2 | 1467-4 | 1467-3 | 1467-2 |
| Unscreened CSF (mL) | 160 | 200 | 295 | 184 | 210 | 275 |
| Specific Energy (MJ/kg) | 5.7 | 5.0 | 4.0 | 4.6 | 4.1 | 3.4 |
| Screened CSF (mL) | 171 | 214 | 305 | 192 | 220 | 292 |
| Reject (% o.d. pulp) | 0.0 | 0.1 | 0.4 | 0.0 | 0.0 | 0.1 |
| Apparent Sheet Density (kg/m³) | 384 | 381 | 353 | 427 | 424 | 413 |
| Burst Index (Kpa · m²/g) | 2.4 | 2.2 | 2.0 | 2.5 | 2.4 | 2.1 |
| Breaking length (km) | 4.5 | 4.3 | 3.8 | 4.7 | 4.6 | 4.1 |
| Tensile Index (N · m/g) | 44.5 | 41.7 | 36.8 | 46.3 | 44.8 | 40.5 |
| Stretch (%) | 1.67 | 1.55 | 1.50 | 1.64 | 1.63 | 1.53 |
| Tear Index (mN · m²/g) (4-Ply) | 5.8 | 6.1 | 5.7 | 5.5 | 5.6 | 5.5 |
| Sheffield Roughness (SU) | 120 | 137 | 186 | 108 | 127 | 159 |
| Brightness (%) | 74 | 75 | 76 | 79 | 78 | 79 |
| Opacity (%) | 86.5 | 86.3 | 85.8 | 84.4 | 83.8 | 84.0 |
| Scattering Coefficient (cm²/g) | 522 | 506 | 506 | 493 | 484 | 495 |
| R - 48 fraction (%) | 44.2 | 46.4 | 49.6 | 39.0 | 42.0 | 45.3 |
| Fines (P-200) (%) | 16.6 | 14.8 | 13.2 | 15.6 | 13.4 | 11.4 |
| W. Weighted Average Fibre Length (mm) | 1.04 | 1.06 | 1.21 | 0.96 | 0.97 | 1.00 |
| L. Weighted Average Fibre Length (mm) | 0.74 | 0.75 | 0.79 | 0.73 | 0.73 | 0.74 |
| Arithmetic Average Fibre Length (mm) | 0.51 | 0.51 | 0.52 | 0.52 | 0.52 | 0.52 |
| | 331-1118 (1) | | | 331-1118 (2) | | |
| | 1468-3 | 1468-2 | 1469-2 | 1471-4 | 1471-3 | 1471-2 |
| Unscreened CSF (mL) | 149 | 191 | 283 | 184 | 223 | 358 |
| Specific Energy (MJ/kg) | 4.2 | 3.8 | 3.0 | 6.5 | 5.5 | 4.3 |
| Screened CSF (mL) | 159 | 200 | 296 | 197 | 240 | 383 |
| Reject (% o.d. pulp) | 0.0 | 0.0 | 0.4 | 0.0 | 0.0 | 0.3 |
| Apparent Sheet Density (kg/m³) | 463 | 458 | 393 | 376 | 358 | 340 |
| Burst Index (Kpa · m²/g) | 2.9 | 2.8 | 2.2 | 2.2 | 2.0 | 1.7 |
| Breaking length (km) | 5.3 | 5.0 | 4.3 | 4.1 | 3.6 | 3.1 |
| Tensile Index (N · m/g) | 51.7 | 49.5 | 41.7 | 40.2 | 35.1 | 30.7 |
| Stretch (%) | 1.90 | 1.83 | 1.65 | 1.69 | 1.41 | 1.25 |
| Tear Index (mN · m²/g) (4-Ply) | 6.0 | 6.0 | 6.3 | 5.6 | 5.6 | 6.1 |
| Sheffield Roughness (SU) | 103 | 113 | 161 | 135 | 164 | 264 |
| Brightness (%) | 77 | 78 | 78 | 77 | 77 | 78 |
| Opacity (%) | 83.3 | 82.0 | 82.3 | 86.2 | 86.2 | 85.3 |
| Scattering Coefficient (cm²/g) | 431 | 429 | 439 | 508 | 520 | 496 |
| R - 48 fraction (%) | 40.5 | 40.8 | 47.6 | 42.3 | 45.7 | 48.5 |
| Fines (P-200) (%) | 13.7 | 13.6 | 12.1 | 17.2 | 15.0 | 14.3 |
| W. Weighted Average Fibre Length (mm) | 0.97 | 0.96 | 1.11 | 1.01 | 1.07 | 1.15 |
| L. Weighted Average Fibre Length (mm) | 0.74 | 0.74 | 0.78 | 0.77 | 0.78 | 0.80 |
| Arithmetic Average Fibre Length (mm) | 0.52 | 0.52 | 0.53 | 0.53 | 0.53 | 0.54 |
| | 331-1122 (1) | | | 331-1126 (1) | | |
| | 1447-4 | 1447-3 | 1448-2 | 1465-4 | 1465-3 | 1465-2 |
| Unscreened CSF (mL) | 210 | 300 | 425 | 191 | 255 | 379 |
| Specific Energy (MJ/kg) | 7.3 | 6.1 | 4.3 | 6.3 | 5.2 | 4.0 |
| Screened CSF (mL) | 227 | 313 | 420 | 202 | 267 | 403 |
| Reject (% o.d. pulp) | 0.1 | 0.2 | 0.7 | 0.0 | 0.0 | 0.2 |
| Apparent Sheet Density (kg/m³) | 360 | 339 | 327 | 363 | 345 | 320 |
| Burst Index (Kpa · m²/g) | 1.7 | 1.6 | 1.4 | 1.8 | 1.7 | 1.4 |
| Breaking length (km) | 3.6 | 3.3 | 2.8 | 3.5 | 3.3 | 2.8 |
| Tensile Index (N · m/g) | 35.8 | 31.9 | 27.2 | 34.8 | 31.9 | 27.1 |
| Stretch (%) | 1.33 | 1.37 | 1.11 | 1.45 | 1.40 | 1.25 |
| Tear Index (mN · m²/g) (4-Ply) | 4.0 | 3.9 | 3.8 | 4.8 | 5.0 | 5.0 |
| Sheffield Roughness (SU) | 144 | 220 | 290 | 164 | 221 | 304 |
| Brightness (%) | 75 | 75 | 76 | 75 | 75 | 76 |
| Opacity (%) | 88.0 | 87.2 | 86.3 | 86.3 | 86.2 | 85.2 |
| Scattering Coefficient (cm²/g) | 533 | 518 | 506 | 505 | 497 | 480 |
| R - 48 fraction (%) | 45.6 | 54.1 | 56.0 | 44.3 | 48.6 | 52.1 |
| Fines (P-200) (%) | 15.8 | 14.0 | 11.3 | 20.3 | 15.8 | 14.9 |
| W. Weighted Average Fibre Length (mm) | 1.00 | 1.06 | 1.25 | 1.08 | 1.10 | 1.24 |
| L. Weighted Average Fibre Length (mm) | 0.75 | 0.78 | 0.84 | 0.85 | 0.87 | 0.90 |
| Arithmetic Average Fibre Length (mm) | 0.49 | 0.52 | 0.52 | 0.58 | 0.58 | 0.59 |
| | 331-1162 (3) | | | 331-1186 (3) | | |
| | 1464-4 | 1464-3 | 1464-2 | 1449-4 | 1449-3 | 1449-2 |
| Unscreened CSF (mL) | 170 | 215 | 266 | 188 | 253 | 380 |
| Specific Energy (MJ/kg) | 5.4 | 4.8 | 4.0 | 7.6 | 6.5 | 5.1 |
| Screened CSF (mL) | 197 | 232 | 291 | 212 | 269 | 382 |
| Reject (% o.d. pulp) | 0.0 | 0.0 | 0.1 | 0.0 | 0.0 | 0.1 |
| Apparent Sheet Density (kg/m³) | 417 | 400 | 394 | 409 | 402 | 354 |
| Burst Index (Kpa · m²/g) | 1.9 | 1.8 | 1.8 | 2.4 | 2.1 | 1.7 |
| Breaking length (km) | 3.7 | 3.6 | 3.3 | 4.3 | 3.8 | 3.4 |
| Tensile Index (N · m/g) | 36.5 | 35.5 | 32.8 | 42.0 | 37.4 | 32.9 |
| Stretch (%) | 1.36 | 1.38 | 1.17 | 1.74 | 1.44 | 1.36 |
| Tear Index (mN · m²/g) (4-Ply) | 5.0 | 5.1 | 4.5 | 5.8 | 5.6 | 5.6 |
| Sheffield Roughness (SU) | 115 | 136 | 163 | 104 | 142 | 226 |
| Brightness (%) | 74 | 74 | 74 | 76 | 77 | 78 |
| Opacity (%) | 88.5 | 87.9 | 87.1 | 86.2 | 85.6 | 84.8 |
| Scattering Coefficient (cm²/g) | 530 | 514 | 518 | 505 | 500 | 499 |
| R - 48 fraction (%) | 45.3 | 45.4 | 46.5 | 46.4 | 48.7 | 51.6 |
| Fines (P-200) (%) | 19.5 | 14.1 | 14.1 | 16.0 | 16.1 | 14.6 |
| W. Weighted Average Fibre Length (mm) | 1.06 | 1.10 | 1.06 | 1.00 | 1.04 | 1.12 |
| L. Weighted Average Fibre Length (mm) | 0.84 | 0.86 | 0.84 | 0.79 | 0.80 | 0.83 |
| Arithmetic Average Fibre Length (mm) | 0.57 | 0.57 | 0.57 | 0.52 | 0.52 | 0.53 |

TABLE XIX
Properties of APRMP Pulps from Hybrid Poplars at a Constant Freeness of 200 mL CSF

| | | | | Length | | | | | | Shef- | | |
|---|---|---|---|---|---|---|---|---|---|---|---|---|
| | Specific | | | Weighted | | | | Tear | | field | | |
| | Refining | R - 48 | Fines | Fibre | Sheet | Tensile | | Index | Bright- | Rough- | Scattering | |
| | Energy | Fraction | (P-200) | Length | Density | Index | Stretch | (mN · | ness | ness | Coefficient | Opacity |
| Hybrid No. | (MJ/kg) | (%) | (%) | (mm) | (kg/m³) | (N · m/g) | (%) | m²/g) | (%) | (SU) | (cm²/g) | (%) |
| 14-129 (1) | 5.9 | 43.5 | 14.0 | 0.78 | 392 | 40.0 | 1.60 | 5.5 | 78 | 130 | 510 | 85.5 |
| 14-129 (2) | 3.7 | 43.2 | 13.9 | 0.78 | 459 | 48.2 | 1.87 | 6.3 | 78 | 111 | 416 | 81.9 |
| 53-242 (1) | 7.0 | 48.0 | 17.4 | 0.81 | 392 | 39.0 | 1.68 | 5.7 | 75 | 128 | 498 | 86.6 |
| 53-242 (2) | 6.9 | 44.5 | 15.2 | 0.77 | 399 | 40.0 | 1.54 | 5.3 | 75 | 113 | 503 | 87.3 |
| 53-246 (1) | 5.2 | 47.3 | 14.5 | 0.81 | 417 | 43.8 | 1.80 | 6.7 | 79 | 118 | 432 | 82.2 |
| 53-246 (2) | 6.7 | 46.5 | 15.8 | 0.82 | 448 | 44.5 | 2.09 | 6.2 | 76 | 95 | 506 | 87.0 |
| 93-968 (1) | 9.3 | 40.0 | 17.4 | 0.85 | 413 | 42.5 | 1.94 | 6.1 | 75 | 90 | 547 | 90.1 |
| 93-968 (2) | 5.9 | 43.3 | 16.3 | 0.79 | 417 | 42.5 | 1.56 | 5.9 | 74 | 95 | 528 | 88.7 |
| 331-1059 (2) | 9.1 | 45.0 | 17.5 | 0.79 | 383 | 40.0 | 1.90 | 5.0 | 75 | 127 | 565 | 88.8 |
| 331-1059 (3) | 9.3 | 48.5 | 17.0 | 0.79 | 383 | 41.2 | 2.06 | 6.2 | 78 | 137 | 550 | 87.4 |
| 331-1061 (1) | 4.6 | 48.6 | 15.1 | 0.84 | 417 | 46.0 | 1.75 | 6.2 | 76 | 103 | 389 | 80.5 |
| 331-1061 (2) | 5.9 | 46.8 | 14.5 | 0.78 | 389 | 44.5 | 1.83 | 5.6 | 76 | 127 | 482 | 85.7 |
| 331-1061 (3) | 7.5 | 47.4 | 16.2 | 0.80 | 361 | 34.8 | 1.30 | 5.0 | 78 | 150 | 494 | 85.4 |
| 331-1062 (1) | 7.2 | 50.4 | 18.3 | 0.87 | 382 | 37.0 | 1.48 | 5.5 | 77 | 107 | 504 | 86.6 |
| 331-1062 (2) | 5.3 | 46.5 | 16.7 | 0.84 | 413 | 42.0 | 1.50 | 5.4 | 77 | 105 | 490 | 85.6 |
| 331-1075 (2) | 11.1 | 38.0 | 19.8 | 0.77 | 361 | 34.0 | 1.40 | 5.0 | 75 | 155 | 580 | 89.5 |
| 331-1093 (1) | 5.0 | 45.6 | 15.7 | 0.75 | 382 | 42.7 | 1.59 | 6.0 | 75 | 132 | 511 | 86.4 |
| 331-1093 (2) | 4.3 | 40.0 | 14.8 | 0.73 | 426 | 45.9 | 1.64 | 5.5 | 79 | 114 | 490 | 84.2 |
| 331-1118 (1) | 3.7 | 40.8 | 13.6 | 0.75 | 448 | 49.5 | 1.83 | 6.0 | 78 | 113 | 429 | 82.0 |
| 331-1118 (2) | 6.0 | 42.5 | 17.2 | 0.77 | 376 | 40.2 | 1.69 | 5.6 | 77 | 137 | 508 | 86.2 |
| 331-1122 (1) | 7.5 | 43.8 | 16.5 | 0.74 | 368 | 37.0 | 1.45 | 4.0 | 75 | 128 | 536 | 88.2 |
| 331-1126 (1) | 6.1 | 44.3 | 20.3 | 0.85 | 363 | 34.8 | 1.45 | 4.8 | 75 | 164 | 505 | 86.3 |
| 331-1162 (3) | 5.0 | 45.3 | 19.5 | 0.85 | 415 | 36.5 | 1.36 | 5.0 | 74 | 115 | 530 | 88.5 |
| 331-1186 (3) | 7.3 | 46.3 | 16.1 | 0.79 | 415 | 43.0 | 1.78 | 5.8 | 76 | 94 | 505 | 86.3 |

Specific Refining Energy

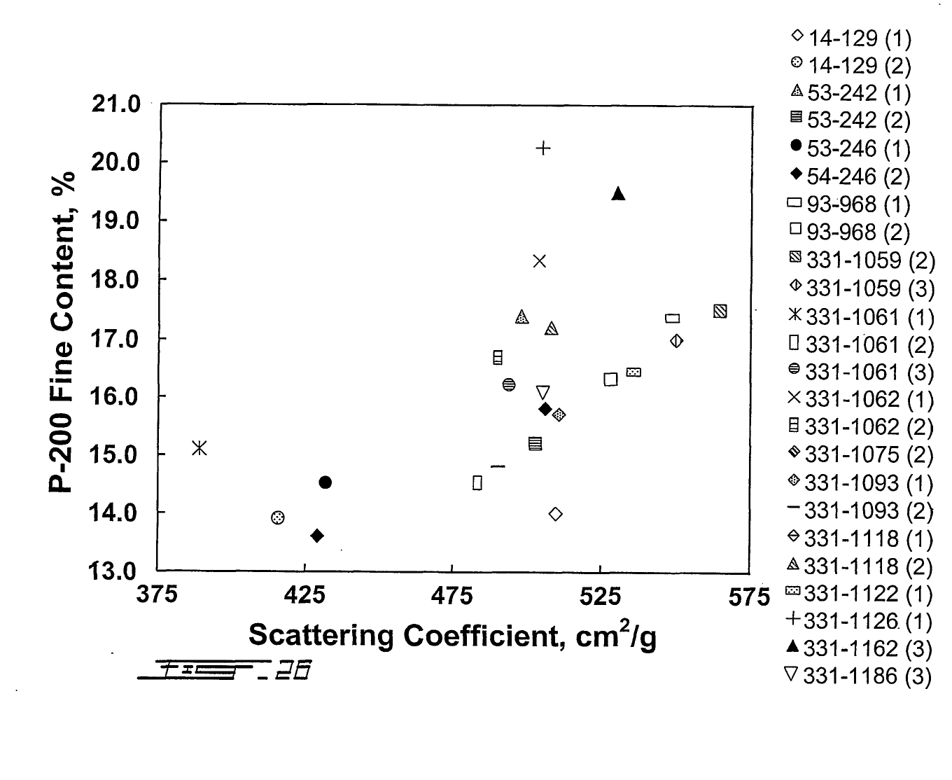

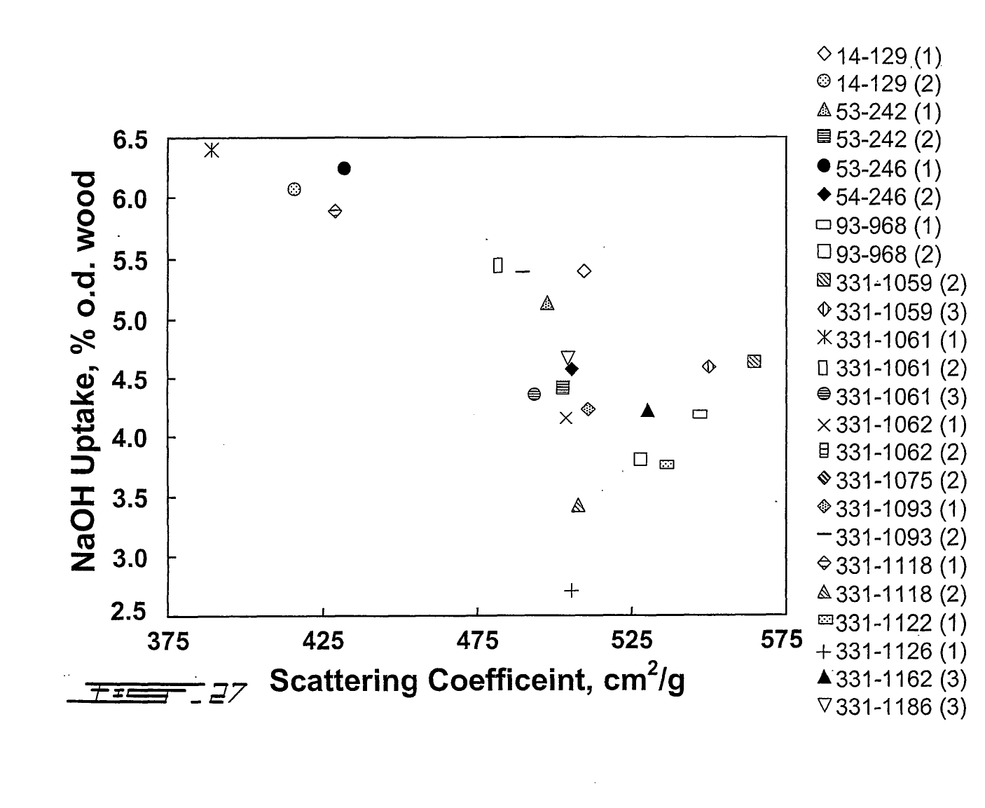

The specific refining energy consumed to reach a given freeness in the range of 150 to 425 mL CSF for the 24 hybrid poplar trees is shown in FIG. 21. The raw data show considerable scatter thanks largely to intraclonal variability which renders clonal effects non-significant (ANOVA p=0.067). Each set of points in FIG. 21 is surrounded by envelopes rather than a best-fit line or curve. The envelopes can be classified into three general groups as shown below.

| High SRE Group | Medium SRE Group | Low SRE Group |
|---|---|---|
| 93-968(1) | 14-129(1) | 14-129(2) |
| 331-1059(2) | 53-242(1) | 53-246(1) |
| 331-1059(3) | 53-242(2) | 331-1061(1) |
| 331-1075(2) | 53-246(2) | 331-1062(2) |
| | 93-968(2) | 331-1093(1) |
| | 331-1061(2) | 331-1093(2) |
| | 331-1061(3) | 331-1118(1) |
| | 331-1062(1) | 331-1162(3) |
| | 331-1118(2) | |
| | 331-1122(1) | |
| | 331-1126(1) | |
| | 331-1186(3) | |

The differences in SRE demand are more evident at 200 mL CSF as clones 93-968(1) and 331-1059(3) require 9.3 MJ/kg SRE whereas clones 14-129(2) and 331-1118(1) require 3.7 MJ/kg SRE or 60% of the energy demand (Table XIX). Clone 331-1075(2) is clearly exceptional as it required 11.1 MJ/kg of specific refining energy to the same freeness level. The three distinct SRE groups shown in FIG. 21 are consistent with previous observations of chemithermomechanical (CTMP) pulping of nine different “wild” aspen clones from Northeast British Columbia.

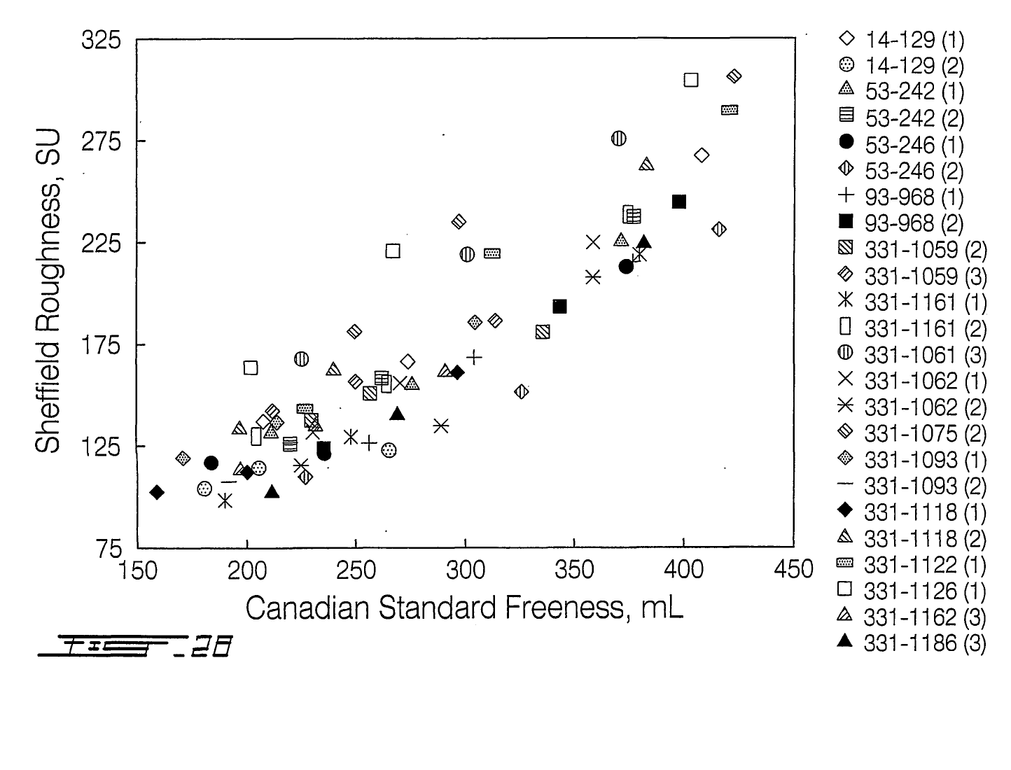

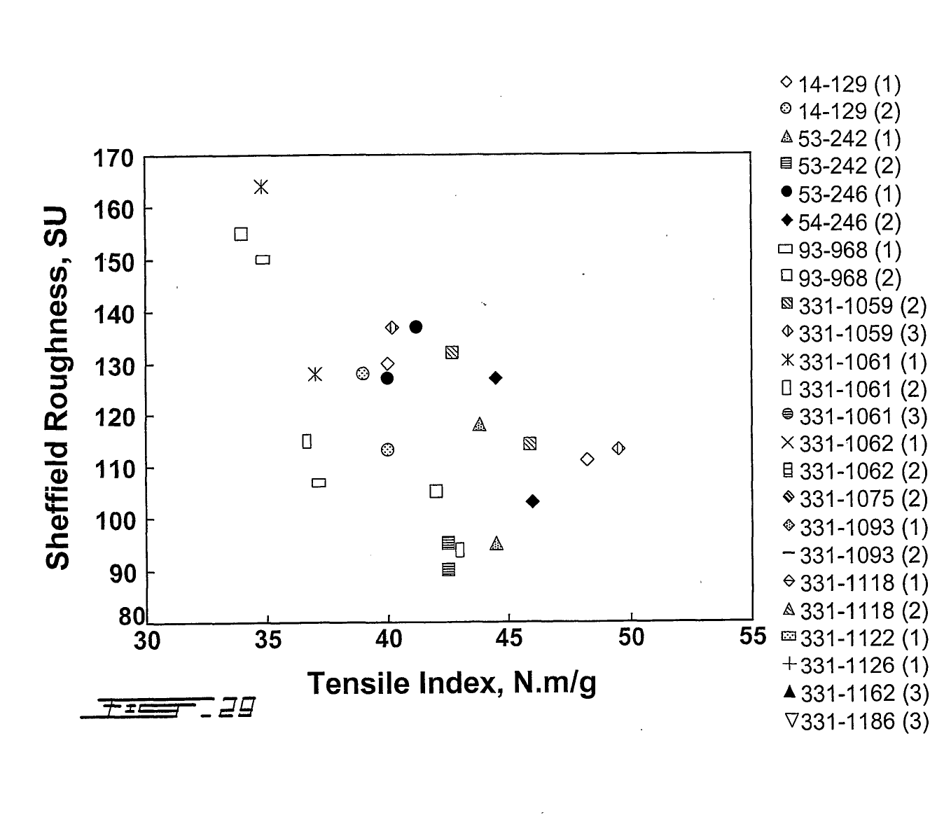

NaOH/H₂O₂ uptake for each tree are shown in Table XX. The data indicate a much lower chemical uptake for the unusual high energy consumption clone 331-1075(2) than for the other clones investigated in this study. NaOH uptake values for each clone at 200 mL CSF are plotted against SRE in FIG. 22. The significant negative relationship noted here (Pearson coefficient −0.526, p=0.025) agrees well with previous findings that SRE of hardwood mechanical pulps increases with diminishing chemical uptake, although the variability seen here is greater than that observed for aspen CTMP pulps. The reasons for intraclonal variability in chemical uptake are not clear. The most probable explanation for low chemical uptake by certain clones is likely a function of the cell wall thickness and lumen diameters of earlywood (large) and latewood (small). It has been reported that a thicker S1 wall makes it more difficult for the hardwood fibre to absorb chemical in order to swell and/or collapse (Marton, R. “Research efforts directed at finding ultimate chemimechanical pulp” Pulp and Paper, 60(6): 81-86 (1986)). A plot of the NaOH uptake vs. chip density (FIG. 23) also confirms previous observations that wood density does not affect chemical uptake by Populus species chips and further contrasts with data suggesting that earlywood density affects chemical uptake for Eucalyptus nitens (Jones, T. G. and Richardson, J. D. “Relationships between wood and chemimechanical pulping properties of New Zealand grown Eucalyptus nitens trees,” Appita J. 52(1), 51-61 (1999)).

TABLE XX
Chip density and chemical uptake for APRMP pulps

| | Chip Density^{a} | NaOH | H₂O₂ |
|---|---|---|---|
| Sample No. | (kg/m³) | (% o.d. wood) | (% o.d. wood) |
| 14-129 (1) | 285 | 5.39 | 3.44 |
| 14-129 (2) | 304 | 6.07 | 3.88 |
| 53-242 (1) | 329 | 5.13 | 3.27 |
| 53-242 (2) | 302 | 4.41 | 2.82 |
| 53-246 (1) | 311 | 6.24 | 3.99 |
| 54-246 (2) | 325 | 4.57 | 2.92 |
| 93-968 (1) | 303 | 4.20 | 2.68 |
| 93-968 (2) | 314 | 3.80 | 2.43 |
| 331-1059 (2) | 303 | 4.63 | 2.95 |
| 331-1059 (3) | 302 | 4.59 | 2.93 |
| 331-1061 (1) | 338 | 6.40 | 4.09 |
| 331-1061 (2) | 328 | 5.41 | 3.46 |
| 331-1061 (3) | 345 | 4.35 | 2.78 |
| 331-1062 (1) | 280 | 4.20 | 2.68 |
| 331-1062 (2) | 290 | 6.51 | 4.24 |
| 331-1075 (2) | 300 | 3.39 | 2.16 |
| 331-1093 (1) | 279 | 4.23 | 2.70 |
| 331-1093 (2) | 288 | 5.38 | 3.43 |
| 331-1118 (1) | 346 | 5.89 | 3.76 |
| 331-1118 (2) | 373 | 3.42 | 2.18 |
| 331-1122 (1) | 283 | 3.80 | 2.43 |
| 331-1126 (1) | 386 | 2.69 | 1.72 |
| 331-1162 (3) | 336 | 4.22 | 2.69 |
| 331-1186 (3) | 292 | 4.69 | 3.00 |

Chip thickness = 2-6 mm

Fibre Properties

As expected, the long-fibre fraction R-48 (retained on the 48-mesh screen of a Bauer-McNett fibre classifier) and LWFL (length-weighted fibre length) increased with increasing freeness and decreasing SRE, whereas the fines content P-200 (passed through the 200-mesh screen of a Bauer McNett fibre classifier) increased with decreasing freeness and increasing SRE as shown in Table XVIII. The LWFL values obtained from the mechanical APRMP pulps at a freeness of 200 mL (Table XIX) show a significant correlation (Pearson coefficient 0.479, p =0.018) with the LWFL values observed for the chemical pulps (Table XII) obtained from the same clones. Unexpectedly, the LWFL values for APRMP pulps were consistently longer than those from the chemical pulps obtained from the same trees. The reasons for this observation is not clear. Perhaps, the alkali treatment of hybrid poplar have softened the middle lamella thus allowing the individual fibres to be peeled from the matrix in a longer and a more intact state in the refiner than those from the chemical pulping process.

Strength Properties and Sheet Consilidation

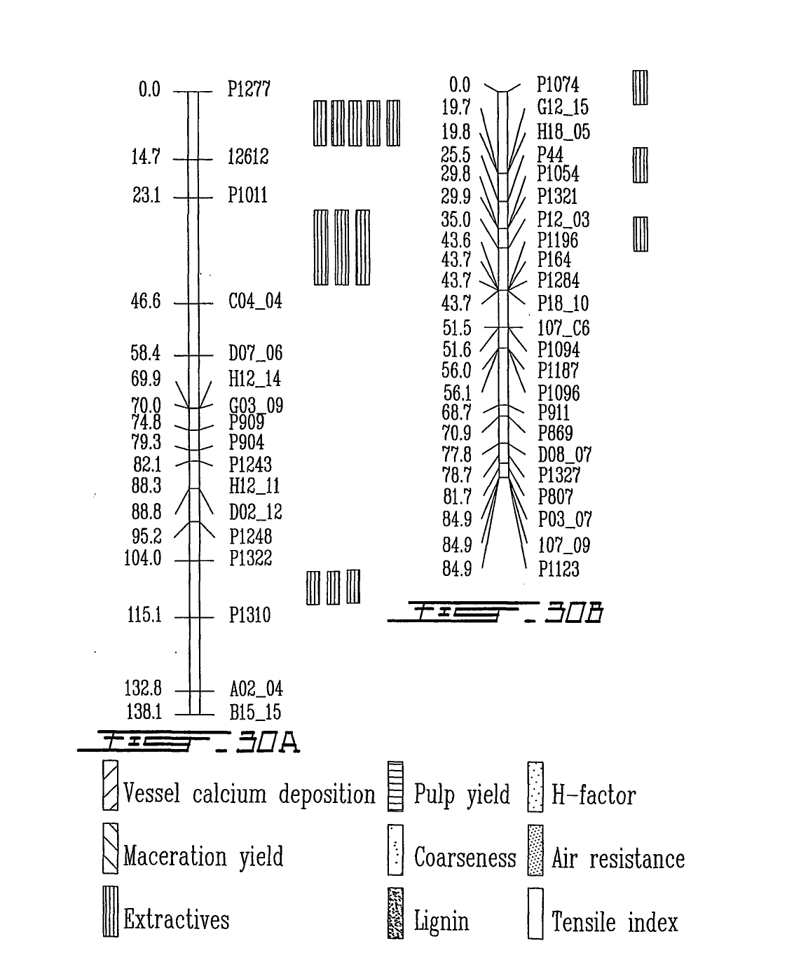

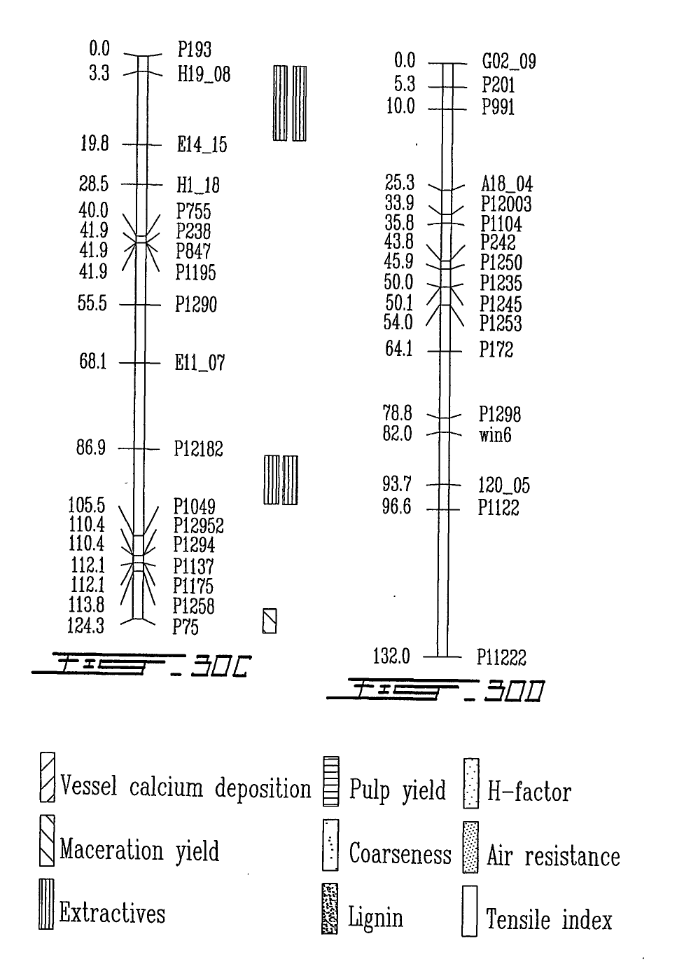

Tensile index increased with decreasing freeness, increasing sheet density, and increasing specific refining energy (Table XVIII). In addition, LWFL also has a highly significant negative relationship with APRMP pulp tensile index (Pearson coefficient −0.74, p=0.001). In general, there is considerable variability in tensile strength from the various clones at a given freeness of 200 mL CSF and a given specific refining energy of 6.0 MJ/kg (Tables XVIII and XXI, respectively). At a given freeness of 200 mL CSF the tensile index values range from 34.0 to 49.5 N·m/g. There is also considerable interclonal variability in tensile strength, for example, the three individuals comprising the genotype clone 331-1061 have a mean tensile index of 41.8 N·m/g with a standard deviation of 5.0 N·m/g at a given freeness of 200 mL CSF (Table XIX). In FIG. 24, NaOH uptake is plotted against tensile index. Again, the data are variable, but it is clear that despite this at a given freeness, increasing chemical uptake results in an increase in tensile strength (Pearson coefficient 0.700, p=0.022). This finding is in good agreement with previous work by Johal et al. and Jackson et al. who found that the tensile indices of aspen CTMP pulps increase with increasing chemical uptake. Intraclonal variation is again the largest component of the variability seen in the tear index data at a given freeness of 200 mL CSF (Table XIX).

TABLE XXI
Properties of APRMP Pulps from Hybrid Poplars at a Constant Specific Energy of 6.0 MJ/kg

| | | Length Weighted | | | | | Scattering |
|---|---|---|---|---|---|---|---|
| | Screened CSF | Fibre Length | Sheet Density | Tensile Index | Tear Index | Sheffield | Coefficient |
| Hybrid No. | (mL) | (mm) | (kg/m³) | (N · m/g) | (mN · m²/g) | Roughness (SU) | (cm²/g) |
| 14-129 (1) | 195 | 0.77 | 390 | 39.5 | 5.5 | 133 | 510 |
| 14-129 (2) | 100 | 0.76 | 490 | 55.0 | 5.8 | 74 | 416 |
| 53-242 (1) | 255 | 0.85 | 375 | 34.5 | 5.8 | 160 | 495 |
| 53-242 (2) | 250 | 0.78 | 388 | 37.0 | 5.4 | 147 | 494 |
| 53-246 (1) | 170 | 0.79 | 442 | 46.7 | 6.7 | 100 | 439 |
| 53-246 (2) | 275 | 0.85 | 410 | 39.0 | 6.3 | 136 | 492 |
| 93-968 (1) | 385 | 0.92 | 360 | 32.8 | 6.2 | 220 | 510 |
| 93-968 (2) | 190 | 0.78 | 412 | 42.0 | 5.9 | 101 | 540 |
| 331-1059 (2) | 400 | 0.84 | 335 | 32.0 | 6.6 | 233 | 476 |
| 331-1059 (3) | 390 | 0.86 | 325 | 32.5 | 6.2 | 226 | 513 |
| 331-1061 (1) | 130 | 0.80 | 445 | 51.5 | 5.9 | 80 | 388 |
| 331-1061 (2) | 194 | 0.78 | 386 | 44.2 | 5.6 | 130 | 482 |
| 331-1061 (3) | 290 | 0.83 | 350 | 31.0 | 5.0 | 232 | 475 |
| 331-1062 (1) | 255 | 0.89 | 364 | 33.0 | 5.6 | 173 | 493 |
| 331-1062 (2) | 165 | 0.83 | 420 | 43.0 | 5.4 | 105 | 511 |
| 331-1075 (2) | 520 | 0.86 | 285 | 22.5 | 3.7 | 387 | 525 |
| 331-1093 (1) | 145 | 0.73 | 393 | 45.5 | 5.8 | 110 | 526 |
| 331-1093 (2) | 125 | 0.72 | 435 | 48.8 | 5.5 | 80 | 504 |
| 331-1118 (1) | 40 | 0.67 | 515 | 55.5 | 5.8 | 78 | 460 |
| 331-1118 (2) | 200 | 0.78 | 367 | 37.8 | 5.6 | 152 | 520 |
| 331-1122 (1) | 310 | 0.79 | 337 | 31.9 | 3.9 | 220 | 512 |
| 331-1126 (1) | 200 | 0.86 | 358 | 34.0 | 4.9 | 178 | 503 |
| 331-1162 (3) | 130 | 0.83 | 435 | 37.6 | 5.3 | 98 | 543 |
| 331-1186 (3) | 290 | 0.81 | 382 | 36.0 | 5.6 | 178 | 500 |

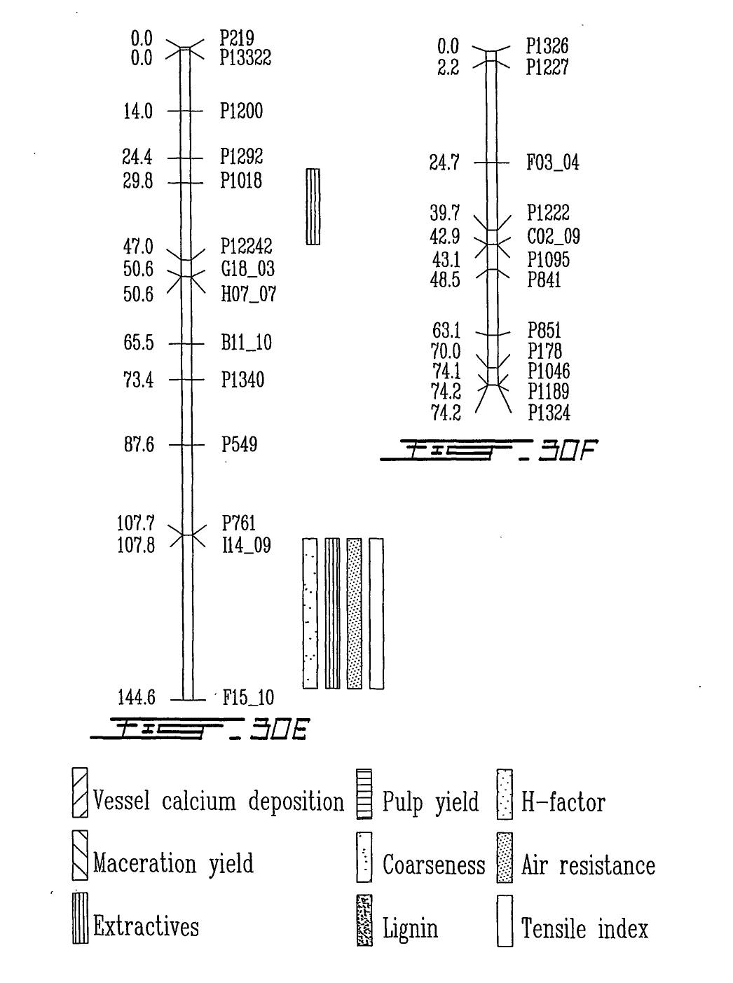

As anticipated, sheet density increases with decreasing freeness and increasing specific refining energy (Table XIX). The extent of the intra- and interclonal variability seen at 200 mL freeness, from 361 kg/m³ to 459 kg/m³, is of the same order as that previously noted for aspen clones and is shown in Table XIX. Whilst some clones (e.g. parent 93-968) produce sheets with similar density properties, others (e.g. parent 14-129) exhibit wide intraclonal variability. The role of alkali uptake at 200 mL freeness in the consolidation of sheet density of hybrid poplar clone APRMP pulps is shown in FIG. 25. The significant positive relationship seen (Pearson coefficient 0.616, p=0.001) indicates the importance of good chemical impregnation to soften fibre cell walls and improve sheet consolidation.

Surface and Optical Properties

As expected, scattering coefficient consistently increased with decreasing freeness and increasing sheet density (Table XVIII). Significant positive correlations were observed between SRE and optical properties scattering coefficient (Pearson coefficient 0.779, p=0.000) and printing opacity (Pearson coefficient 0.738, p=0.003).

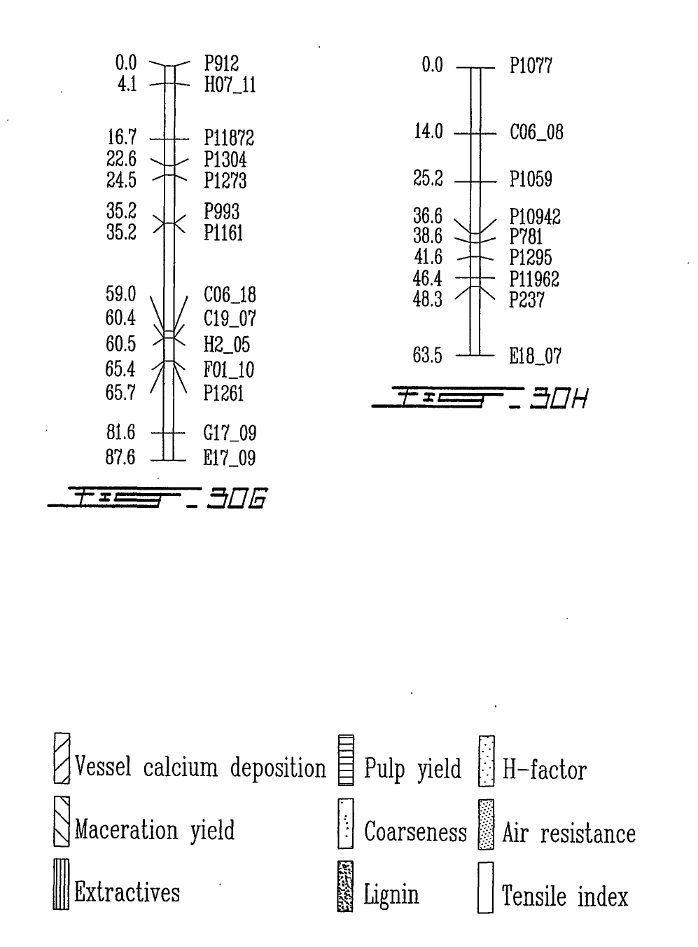

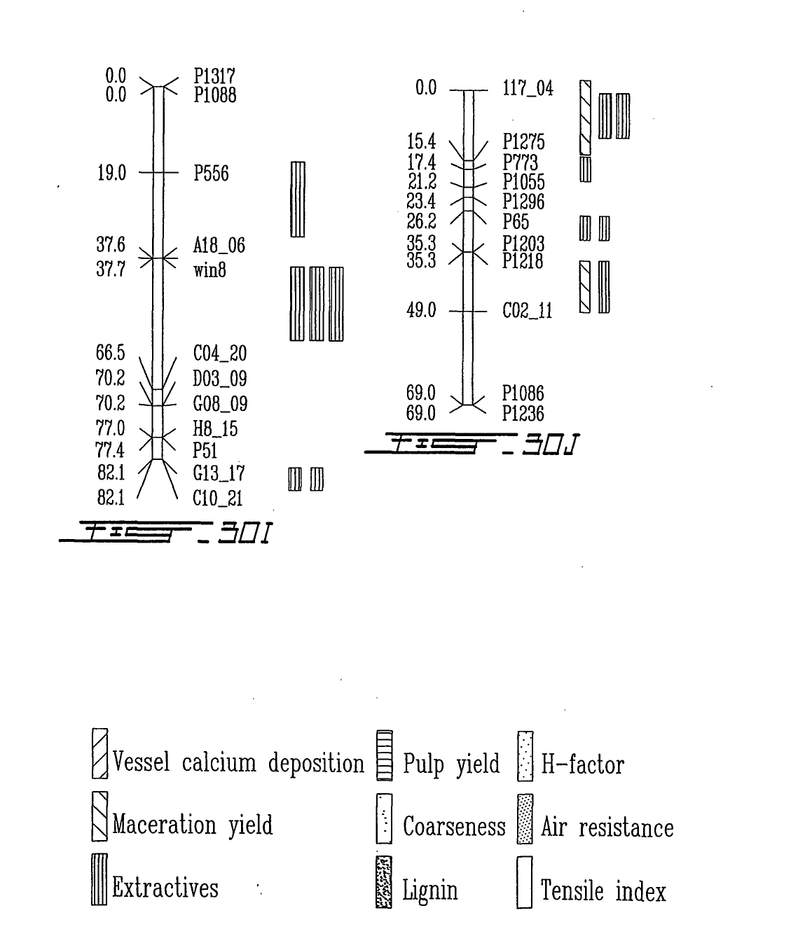

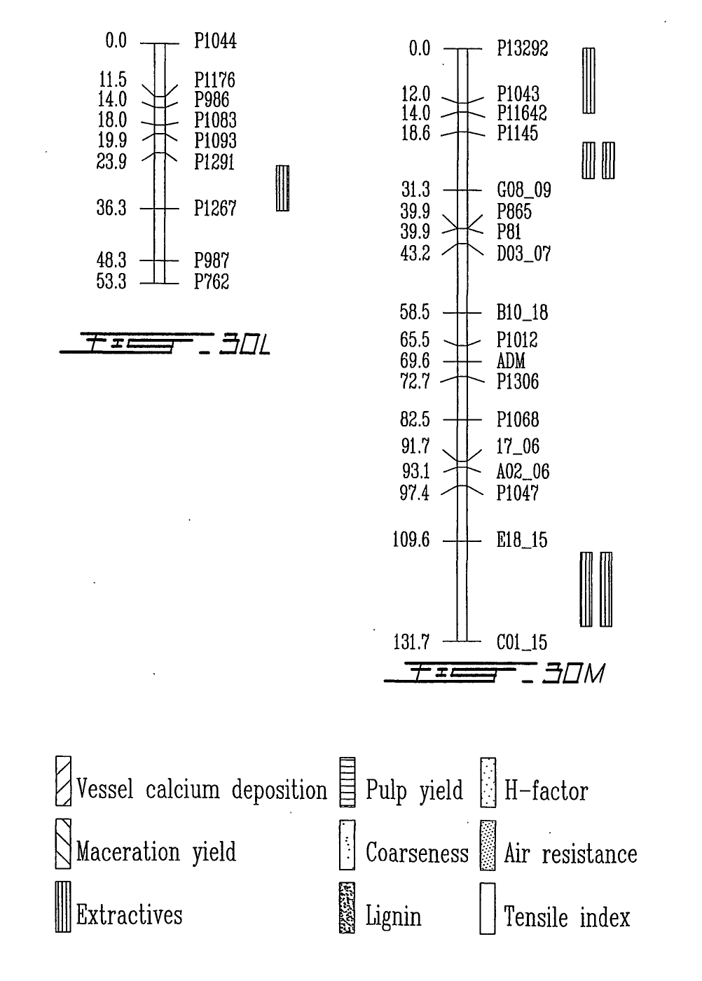

In FIG. 26, the fines content (P-200) is shown as a function of scattering coefficient. The significant positive relationship (Pearson coefficient 0.637, p=0.001) confirms previous observations for aspen in that those clones with the highest fines content also exhibit high scattering coefficients and high opacity values. The negative effect of chip alkali uptake—on light scattering development is indicated in FIG. 27 (Pearson coefficient −0.713, p=0.000). The most probable explanation for this negative effect is that increased alkali uptake makes the fibre separation at the middle lamella easier and thus producing fewer fines. Secondly, the higher alkali uptake makes the fibres more flexible and hydrophilic thus resulting in more fibre bonding and reduced light scattering.

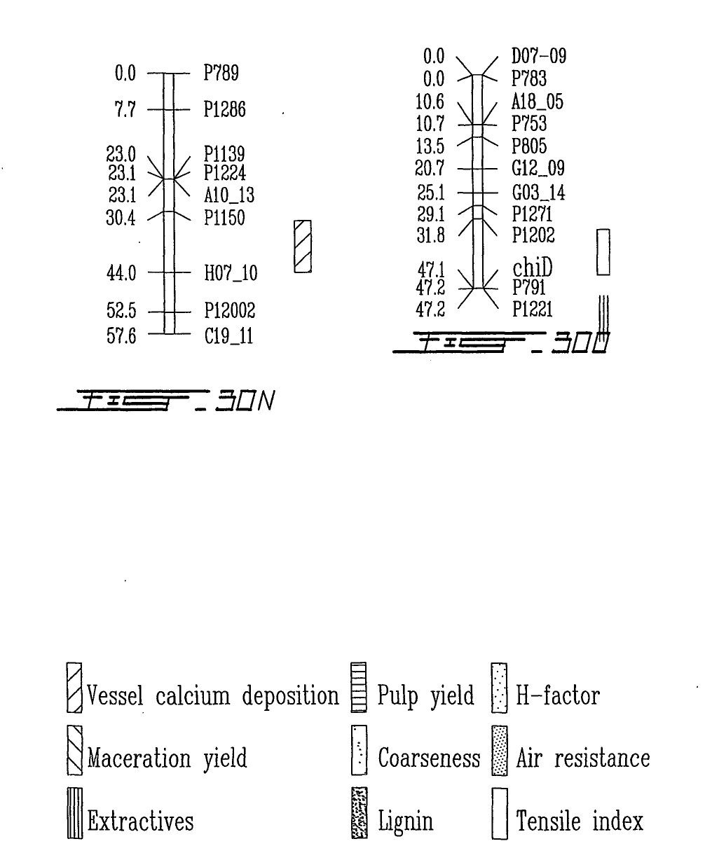

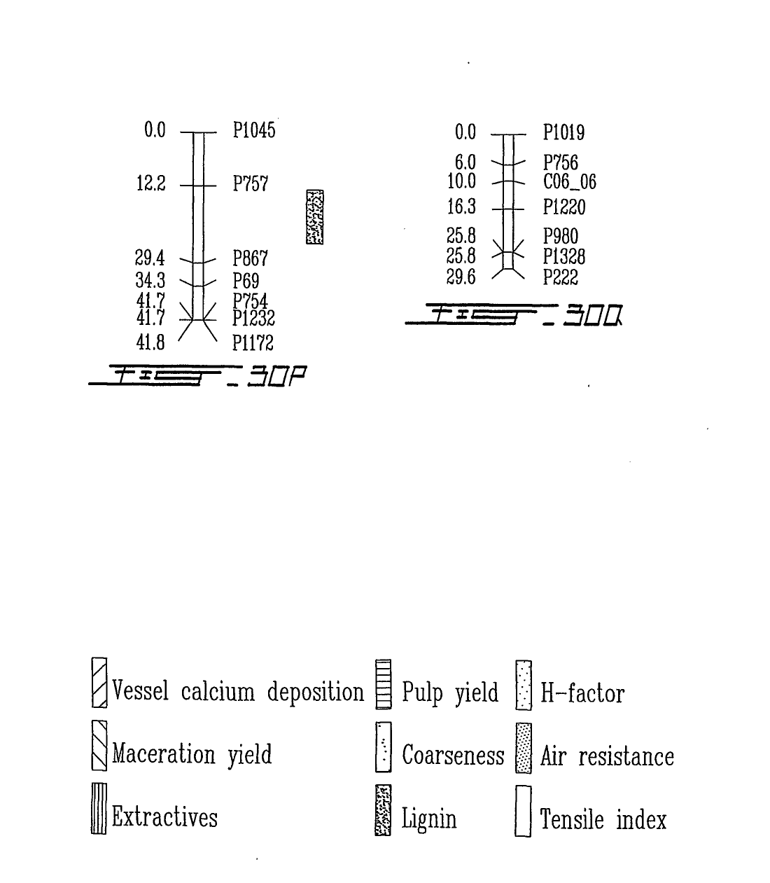

Sheffield roughness increased with increasing freeness (FIG. 28). The plot of Sheffield roughness vs. tensile strength (FIG. 29) indicates that at high tensile index, most clones exhibit excellent sheet surface properties. The significant negative relationship seen (Pearson coefficient −0.602, p=0.002) does not alter the fact that, within this hybrid population, a wide variety of pulp strengths can be had whilst maintaining a constant smoothness level (see Table XXII).

TABLE XXII
Interclonal variability of strength properties
for given formation properties

| | Tensile index | Sheffield Smoothness |
|---|---|---|
| Clone | (N · m/g) | (SU) |
| 331-1118(1) | 49.5 | 113 |
| 331-1162(3) | 36.5 | 115 |

The brightness of the APRMP pulps from different clones under significantly variable H₂O₂ uptake was surprisingly similar. A tight range of brightness values was obtained from the hybrid poplar pulps, from 74-79%. This compares very well with previous brightness results for aspen clones which showed greater variability over a lower spectrum of values, from 49-69%. The aspen values may be explained by the occurrence in natural stands of highly stained wood and by wide differences in the lignin content of the examined trees.

QTL Mapping Using Pulp Properties Phenotypic Data

For most of the pulping parameters examined in this study, both intra- and interclonal factors were significant determinators of the population variability encountered. This, coupled with the necessarily small sample size utilized, makes the correlation of genotypic and phenotypic variability statistically challenging. Some data sets did yield significant QTL detections—for example, a putative QTL has been found for H-factor with a LOD score of 4.04 (see FIG. 29 and Table XXIII). Importantly using the kraft pulping data, a significant QTL for tensile index (LOD score 3.48) and a less significant QTL for air resistance (LOD score 2.62) were detected in a chromosomal position coincident with that detected for fibre coarseness and microfibril angle. These results are depicted in Table XXIII. These data suggest that not only does this genetic region contain genes which affect multiple related pulp parameters and is therefore worthy of further investigation, but that the coarseness values obtained from the peracetic acid maceration/FQA fibre analysis technique do indeed accurately reflect the performance of the pulp in terms of a number of important parameters. The observation strongly supports the use of this procedure as a technique for rapid assessment of tree populations for wood quality.

TABLE XXIII
Significant QTL detected for H factor

| | | LOD | Phen | | | |
|---|---|---|---|---|---|---|
| Trait | Marker/Linkage | Score | % | Length/cM | Weight | Dom. |
| H factor | PAL2-P214/Y | 4.04 | 95.6 | 6.6 | 169.83 | −337.80 |
| Tensile index | I14_09-F15_10/E | 3.48 | 87.2 | 37.3 | 1.5378 | 9.8668 |
| Air resistance | I14_09-F15_10/E | 2.62* | 88.4 | 37.3 | 519.36 | −250.13 |
| (Gurley) | | | | | | |
| Fibre | I14_09-F15_10/E | 3.49 | 55.9 | 37.3 | 72.794 | −79.906 |
| Coarseness/MFA** | | | | | | |

Reported due to significant location.
**Data from Table I repeated to illustrate co-localization with other reported QTL.

Most of the QTL found, however, had LOD significance scores of approximately the threshold value of 2.90 or lower, indicating a high possibility of spurious detection. QTL mapping of these data is, therefore, not presented here as the data sets are simply not extensive enough for statistical significance. These data will form part of a larger and continuing study on this population of hybrid poplars with the eventual goal of genetic mapping of specific pulping and papermaking characteristics. This is considered to be an important outcome as, as has been clearly shown by this and numerous other reports, it is often highly problematic to accurately predict pulp and papermaking properties from easily measured parameters such as fibre properties, wood density, etc. To actually determine the pulp and paper properties of a clone, it is still necessary to pilot pulp the entire stem. It is anticipated that QTL mapping of a large enough sample set of pilot pulps will enable the detection of the particular subset of genes which directly affect pulp and paper parameters and the development of rapid assessment methods for those properties of immediate industrial value. This study represents the first steps towards eventual achievement of this highly important objective.

QTL Mapping

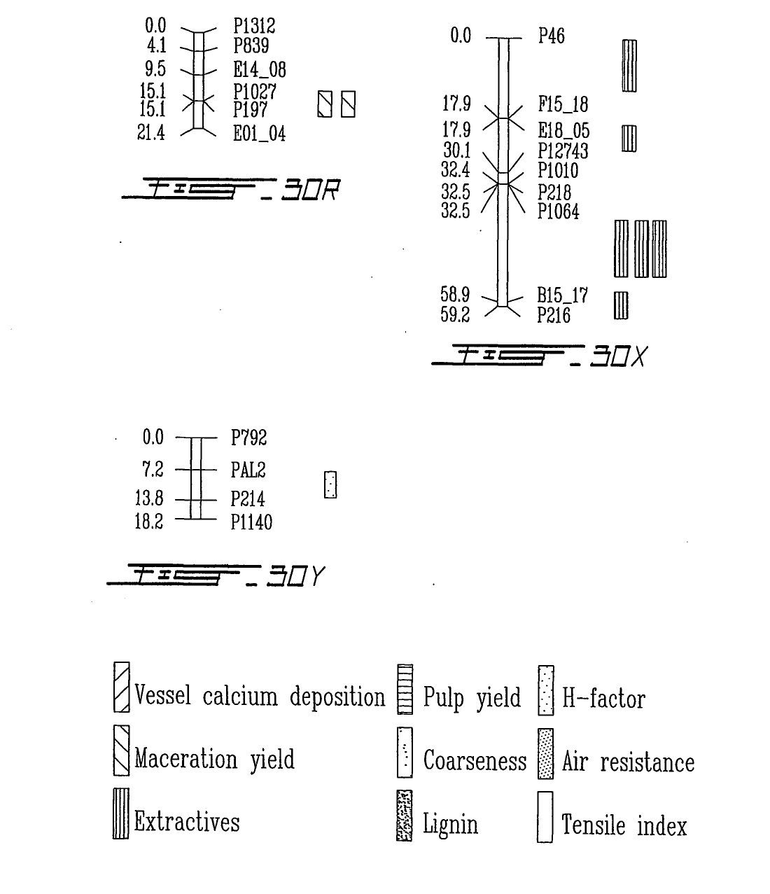

FIG. 30 illustrates the current status of QTL mapping using the Family 331 hybrid poplar mapping pedigree. The map shows the 19 linkage groups that are approximately equivalent to the 19 Populus chromosomes as vertical bars labelled A-Y as obtained from the University of Washington. Positions of assigned RFLP, RAPD and STS markers are indicated on each linkage group. Assigned QTL regions for each of the traits examined in the study are indicated as colour-coded bars adjacent to the linkage groups. Details on the significance of the QTL and the genetic distances they cover can be found in the appropriate tables, although it is important to note that—with the single exception of kraft pulp yield—each reported QTL exceeds the 95% statistical confidence level, as determined by the LOD threshold score of 2.9.

RAPD Analysis and Polymorphic Product Characterization

Table XXIV shows the screened suite of markers associated with the QTL linked to the specific traits of interest examined in this study. Each of these RAPD/RFLP markers was used in a PCR reaction to generate a polymorphic product from the phenotypically selected F₂ generation individuals indicated. Table XXIV also presents the number of sequences generated from the polymorphic bands isolated. Proposed functionalities for the sequences, based on similarities to sequences already in public databases, are also shown. The polymorphic marker bands have been fully or partially sequenced and functionality has been assigned according to homology with previously published sequences on public databases (e.g. Genbank).

By sequence homology it will now be possible to identify orthologous functional genes in trees of the genus Populus, Picea, Betula, Abies, Larix, Taxus, Ulmus, Prunus, Quercus, Malus, Arbutus, Salix, Platanus, Acer, Tsuga, Pseudotsuga, Pinus, Fraxinus, Eucalytpus, Acacia, Abrus, Cupressus, Fagus, Juniperus, Thuja, Canya.

TABLE XXIV
Markers associated with QTLs linked to specific traits

| | | # | Product | |
|---|---|---|---|---|
| Trait | Marker | Sequences | Size (bp) | Database ID |
| Maceration | I17_04 | 2 | | AC007018 Arabidopsis thaliana |
| yield | | | | chromosome; AP002820) putative |
| | | | | transposable element Tip100 protein RICE |
| Maceration | G02_11 | 5 | 1138, 990, | (AC006136) putative retroelement pol |
| yield | | | 1032, 976, | polyprotein [Arabidopsis]; AC009400) |
| | | | 986 | hypothetical protein [Arabidopsis thaliana; |
| | | | | >gi\|13241678\|gb\|AAK16420.1] |
| | | | | (AF320086) RIRE gag/pol protein [Zea |
| | | | | mays]; unknown; AC020580) hypothetical |
| | | | | protein, 3′partial |
| Yield/H- | E01_04 | 3 | 347, 334, | (AC002332) hypothetical protein |
| factor | | | 356 | [Arabidopsis thaliana]; AC007357) |
| | | | | F3F19.15 [Arabidopsis thaliana]; |
| | | | | (AB024037) |
| | | | | emb\|CAB77928.1˜gene_id:MSK10.2˜simi- |
| | | | | lar to unknown |
| Yield/H- | P1027 | 3 | 539, 589, | hypothetical protein, At; putative |
| factor | | | 593 | retroelement; At EST ATTS1136, putative |
| | | | | disease resistance gene. |
| Lignin | P757 | 2 | 281, 199 | Arabidopsis retrotransposon-like protein, |
| | | | | Z97342. |
| Coarseness/ | I14_09 | 3 | 545, 545, | unknown; low hits: cotton fad aj244890; |
| microfibril | | | 869 | poplar agamous (64% in 197 nt); copia-like |
| angle/ | | | | polyprotein [Arabidopsis thaliana] |
| tensile | | | | |
| index/air | | | | |
| resistance | | | | |
| | F15_10 | 2 | 950, 980 | unknown Arabidopsis gene; |
| | | | | Many proline-rich proteins (#1 = cicer |
| | | | | arietinium), +3 frame |
| Extractives | B15 | 2 | 1756, | endo-1,4-betaglucanase, fibronectin |
| | | | 1693 | repeat signature |
| | H19_08 | 1 | 810 | transformer-SR ribonucleoprotein |
| | G13_17 | 2 | 1400, 1628 | several dnaJ-like protein [Arabidopsis |
| | | | | thaliana]; |
| | | | | gi\|1491720\|emb\|CAA67813.1\| (X99451) |
| | | | | extensin-like protein Dif10 [Lycopersicon |
| | | | | esculentum |
| | P1054 | 1 | 787 | Cicer arietinum mRNA for glucan-endo- |
| | | | | 1,3-beta-glucosidase |
| | P1018 | 1 | 522 | AC007191 Arabidopsis thaliana |
| | | | | chromosome |
| | H12 | 3 | 332, 386, | hypothetical protein (COP1 regulatory), |
| | | | 350 | endoglucanase, 3-oxo-5-alpha-steroid-4- |
| | | | | dehydrogenase. |
| Calcium | H07_10 | 3 | 977, 978, | (AC003970) Similar to Glucose-6- |
| deposition | | | 754 | phosphate dehydrogenases, At; AC006267) |
| | | | | putative polyprotein [Arabidopsis |
| | | | | thaliana]; (AC006267) putative |
| | | | | polyprotein [Arabidopsis thaliana] |

1. A method of identifying a gene in tree of a second genus and/or species capable of expressing desired biological and/or biochemical phenotypes, said second tree genus and/or species being of different genus and/or species than a first tree species, comprising the steps of:
a) obtaining a nucleic acid sample from tree of a first genus and/or species and/or hybrid thereof;
b) obtaining either a restriction fragment length polymorphism (RFLP) pattern or PCR-fingerprint for said first tree by subjecting said nucleic acid of step a) to at least one restriction enzyme and/or standard PCR conditions with at least one specific primer;
c) correlating said RFLP pattern or PCR-fingerprint of step b) to at least one selected biological and/or biochemical phenotype of said first tree genus and/or species wherein said phenotype is associated with a genetic locus identified by and/or associated with said RFLP pattern or PCR fingerprint; and
d) identifying orthologous functional gene by sequence homology in the second tree genus and/or species.

2. The method according to claim 1, which further comprises a step i) after step c):
i) correlating said RFLP pattern or PCR-fingerprint of step b) to said at least one selected biological and/or biochemical phenotype of said first tree genus and/or species to said at least one selected biological and/or biochemical phenotype of said second tree genus and/or species.

3. The method according to claim 1, wherein said gene in said second tree genus and/or species is orthologous to said gene in said first tree genus and/or species.

4. The method according to claim 1, wherein said first tree genus and said second tree genus are selected from the group consisting of Populus, Picea, Betula, Abies, Larix, Taxus, Ulmus, Prunus, Quercus, Malusj Arbutus, Salix, Platanus, Acer, Tsuga, Pseudotsuga, Pinus, Fraxinus, Eucalyptus, Acacia, Abrus, Cupressus, Fagus, Juniperus, Thuja and Canya.

5. The method according to claim 1, wherein said first tree species and said second tree species are independently selected from the group consisting of Populus trichocarpa, Populus deltoides, Populus tremuloides and a hybrid thereof, wherein said first tree species is different from said second tree species.

6. The method according to claim 1, wherein said first species is selected from the group consisting of Populus trichcarpa (clone 93-968), Populus deltoides (clone ILL-129), and a hybrid thereof.

7. The method according to claim 1, wherein said first tree species is a pure species.

8. The method according to claim 1, wherein said first tree species is a hybrid.

9. The method according to claim 8, wherein said hybrid is a hybrid poplar.

10. The method according to claim 9, wherein said hybrid poplar is Populus trichocarpa X Populus deltoides—Family 331.

11. The method according to claim 1, wherein said PCR-fingerprint is selected from the group consisting of RAPD, AFLP, CAP and SCAR.

12. The method according to claim 1, wherein said identified markers are selected from the group consisting of I17_{—}04, G02_{—}11, E01_{—}04, P1027, P757, I14_{—}09, F15_{—}10, B15, H19_{—}08, G13_{—}17, P1054, P1018, H12, H07_{—}10 and G03.

13. The method according to claim 1, wherein said correlating of step c) further comprises sequencing of polymorphic DNA sequences associated with a genetic locus associated with said phenotype.

14. The method according to claim 1, wherein polymorphic DNA sequences represent candidate genes or are highly linked to candidate genes for use as DNA markers as in step c).

15. The method according to claim 1, wherein polymorphic DNA sequences are physically and/or genetically linked to candidate genes.

16. The method according to claim 1, wherein said first genus and/or species and/or hybrid thereof is naturally or artificially produced.

17. The method according to claim 1, wherein said second genus and/or species and/or hybrid thereof is naturally or artificially produced.

18. The method according to claim 1, wherein said sample of step a) is obtained from a leaf, cotyledon, cambium, root, bud, stem, cork, phloem, flower or xylem.

19. A method of identifying a genetic marker in tree of a second genus and/or species associated with a genetic locus conferring at least one enhanced property selected from the group consisting of fiber length, fiber coarseness, DBH (diameter at breast height), microfibril angle, density, pulp strength, pulp yield, lignin content, pitch propensity, air resistance, kraft pulping H-factor, specific refining energy, wood extractive compounds content and calcium accumulation, said second tree genus and/or species being of different genus and/or species than a first tree species, which comprises the steps of:
a) obtaining a sexually mature parent tree of said first genus and/or species and/or hybrid thereof exhibiting enhanced properties;
b) obtaining a plurality of progeny trees of said parent tree by performing self or cross-pollination;
c) assessing multiple progeny trees for each of a plurality of genetic markers;
d) identifying a genetic marker segregating in an essentially Mendelian ratio and showing linkage with at least some other of said plurality of genetic markers;
e) measuring at least one of said properties in multiple progeny trees; and
f) correlating the presence of enhanced property with a least one marker identified in step d) as segregating in an essentially Mendelian ratio and showing linkage with at least some of said other markers, the correlation of the presence of enhanced properties with a marker indicating that said marker is associated with a genetic locus conferring enhanced property.

20. The method of claim 19, which further comprises steps i) and j) after step b:
i) obtaining a nucleic acid sample from said mature parent tree;
j) obtaining either a restriction fragment length polymorphism (RFLP) pattern or PCR-based fingerprint for said parent tree by subjecting said nucleic acid of step i) to at least one restriction enzyme and/or standard PCR conditions with at least one specific primer

21. The method of claim 19, further comprising constructing a genetic linkage map of said parent tree using said plurality of genetic markers.

22. The method of claim 21, wherein said genetic linkage map is a QTL map.

23. The method of claim 19, wherein said genetic marker is a restriction fragment length polymorphism (RFLPs) and/or a PCR-based marker.

24. The method of claim 19, wherein said genetic marker is selected from the group consisting of RAPD, AFLP, CAP, SNP, STS and SCAR.

25. The method of claim 19, wherein at least one of said genetic markers is correlated with a locus or with a quantitative traits loci (QTLs).

26. The method according to claim 19, wherein said genetic marker in said second tree genus and/or species is orthologous to said genetic marker in said first tree genus and/or species.

27. The method according to claim 19, wherein said first tree genus and said second tree genus are selected from the group consisting of Populus, Picea, Betula, Abies, Larix, Taxus, Ulmus, Prunus, Quercus, Malus, Arbutus, Salix, Platanus, Acer, Tsuga, Pseudotsuga, Pinus, Fraxinus, Eucalyptus, Acacia, Abrus, Cupressus, Fagus, Juniperus, Thuja and Canya.

28. The method according to claim 19, wherein said first tree species and said second tree species are independently selected from the group consisting of Populus trichocarpa, Populus deltoides, Populus tremuloides and a hybrid thereof, wherein said first tree species is different from said second tree species.

29. The method according to claim 19, wherein said first species is selected from the group consisting of Populus trichcarpa (clone 93-968), Populus deltoides (clone ILL-129), and a hybrid thereof.

30. The method according to claim 19, wherein said first tree species is a pure species.

31. The method according to claim 19, wherein said first tree species is a hybrid.

32. The method according to claim 31, wherein said hybrid is a hybrid poplar.

33. The method according to claim 32, wherein said hybrid poplar is Populus trichocarpa X Populus deltoides—Family 331.

34. The method according to claim 19, wherein said identified markers are selected from the group consisting of I17_{—}04, G02_{—}11, E01_{—}04, P1027, P757, I14_{—}09, F15_{—}10, B15, H19_{—}08, G13_{—}17, P1054, P1018, H12, H07_{—}10 and G03.

35. The method according to claim 19, wherein said genetic markers represent candidate genes or are highly linked to candidate genes.

36. The method according to claim 19, wherein said genetic markers are physically and/or genetically linked to candidate genes.

37. The method according to claim 19, wherein said first genus and/or species and/or hybrid thereof is naturally or artificially produced.

38. The method according to claim 19, wherein said second genus and/or species and/or hybrid thereof is naturally or artificially produced.

39. The method according to claim 19, wherein said sample of step i) is obtained from a leaf, cotyledon, cambium, root, bud, stem, cork, phloem, flower or xylem.

40. The method of claim 19, wherein said parent tree is the seed parent tree to each of said progeny trees.

41. A method of using a genetic marker for producing a plurality of clonal trees that have at least one enhanced property selected from the group consisting of fiber length, fiber coarseness, DBH (diameter at breast height), microfibril angle, density, pulp strength, pulp yield, lignin content, pitch propensity air resistance, kraft pulping H-factor, specific refining energy, wood extractive compounds content and calcium accumulation, which comprises the steps of:
a) obtaining a sexually mature first parent tree exhibiting enhanced property;
b) obtaining a plurality of progeny trees of said parent tree by performing self or cross-pollination;
c) assessing multiple progeny trees for each of a plurality of genetic markers identified as associated with a genetic locus conferring at least one enhanced property in a second tree of a different genus and/or species than said first parent tree;
d) identifying those genetic markers segregating in an essentially Mendelian ratio in multiple progeny trees;
e) correlating the presence of enhanced property in multiple progeny trees with a least one marker identified in step d);
f) selecting a progeny tree containing a marker identified in step f) as associated with a genetic locus conferring enhanced property; and
g) vegetatively propagating said progeny tree selected in step g) to produce a plurality of clonal trees.

42. The method of claim 41, wherein said each of genetic markers identified as associated with a genetic locus conferring at least one enhanced property in a second tree of a different genus and/or species than said first parent tree is identified according to the method of claim 18.

43. The method of claim 41, further comprising constructing a genetic linkage map of said first parent tree using said plurality of genetic markers.

44. The method of claim 41, wherein said genetic linkage map is a QTL map.

45. The method of claim 41, wherein said genetic markers are restriction fragment length polymorphisms (RFLPs) and/or PCR-based markers.

46. The method of claim 41, wherein said genetic marker is selected from the group consisting of RAPD, AFLP, CAP, SNP, STS and SCAR.

47. The method of claim 41, wherein at least one of said genetic markers is correlated with a locus or with a quantitative traits loci (QTLs).

48. The method according to claim 41, wherein said genetic marker in said second tree genus and/or species is orthologous to said genetic marker in said first tree genus and/or species.

49. The method according to claim 41, wherein said first tree genus and said second tree genus are selected from the group consisting of Populus, Picea, Betula, Abies, Larix, Taxus, Ulmus, Prunus, Quercus, Malus, Arbutus, Salix, Platanus, Acer, Tsuga, Pseudotsuga, Pinus, Fraxinus, Eucalyptus, Acacia, Abrus, Cupressus, Fagus, Juniperus, Thuja and Canya.

50. The method according to claim 41, wherein said first tree species and said second tree species are independently selected from the group consisting of Populus trichocarpa, Populus deltoides, Populus tremuloides or hybrid thereof, wherein said first tree species is different from said second tree species.

51. The method according to claim 41, wherein said first species is selected from the group consisting of Populus trichcarpa (clone 93-968), Populus deltoides (clone ILL-129), or hybrid thereof.

52. The method according to claim 41, wherein said first tree species is a pure species.

53. The method according to claim 41, wherein said first tree species is a hybrid.

54. The method according to claim 53, wherein said hybrid is a hybrid poplar.

55. The method according to claim 54, wherein said hybrid poplar is Populus trichocarpa X Populus deltoides—Family 331.

56. The method according to claim 41, wherein said identified markers are selected from the group consisting of I17_{—}04, G02_{—}11, E01_{—}04, P1027, P757, I14_{—}09, F15_{—}10, B15, H19_{—}08, G13_{—}17, P1054, P1018, H12, H07_{—}10 and G03.

57. The method according to claim 41, wherein said genetic markers represent candidate genes or are highly linked to candidate genes.

58. The method according to claim 41, wherein said genetic markers are physically and/or genetically linked to candidate genes.

59. The method according to claim 41, wherein said first genus and/or species and/or hybrid thereof is naturally or artificially produced.

60. The method according to claim 41, wherein said second genus and/or species and/or hybrid thereof is naturally or artificially produced.

61. The method according to claim 41, wherein DNA for identifying said genetic markers is obtained from a leaf, cotyledon, cambium, root, bud, stem, cork, phloem, flower or xylem.

62. The method of claim 41, wherein said parent tree is the seed parent tree to each of said progeny trees.

63. A stand of clonal enhanced property trees produced by the method of claim 41, the genome of said trees containing the same genetic marker associated with a genetic locus conferring at least one enhanced property in said second tree of a different genus and/or species than said first parent tree.

64. A method of using a genetic marker for producing a family of trees wherein at least about half exhibit at least of enhanced property selected from the group consisting of fiber length, fiber coarseness, DBH (diameter at breast height), microfibril angle, density, pulp strength, pulp yield, lignin content, pitch propensity, air resistance, kraft pulping H-factor, specific refining energy, wood extractive compounds content and calcium accumulation, which comprises the steps of:
a) obtaining a sexually mature first parent tree exhibiting enhanced property;
b) obtaining a plurality of progeny trees of said parent tree by performing self or cross-pollination;
c) assessing multiple progeny trees for each of a plurality of genetic markers identified as associated with a genetic locus conferring at least one enhanced property in a second tree of a different genus and/or species than said first parent tree;
d) identifying those genetic markers segregating in an essentially Mendelian ratio in multiple progeny trees;
e) correlating the presence of enhanced property in multiple progeny trees with a least one marker identified in step d);
f) selecting a progeny tree containing a marker identified in step f) as associated with a genetic locus conferring enhanced property; and
g) sexually propagating said progeny tree selected in step g) to produce a family of trees, at least about half of said family of trees containing a genetic locus conferring enhanced property and said family of trees exhibiting enhanced property.

65. The method of claim 64, wherein said each of genetic markers identified as associated with a genetic locus conferring at least one enhanced property in a second tree of a different genus and/or species than said first parent tree is identified according to the method of claim 18.

66. The method of claim 64, further comprising constructing a genetic linkage map of said first parent tree using said plurality of genetic markers.

67. The method of claim 64, wherein said genetic linkage map is a QTL map.

68. The method of claim 64, wherein said genetic markers are restriction fragment length polymorphisms (RFLPs) and/or PCR-based markers.

69. The method of claim 64, wherein said genetic marker is selected from the group consisting of RAPD, AFLP, CAP, SNP, STS and SCAR.

70. The method of claim 64, wherein at least one of said genetic markers is correlated with a locus or with a quantitative traits loci (QTLs).

71. The method according to claim 64, wherein said genetic marker in said second tree genus and/or species is orthologous to said genetic marker in said first tree genus and/or species.

72. The method according to claim 64, wherein said first tree genus and said second tree genus are selected from the group consisting of Populus, Picea, Betula, Abies, Larix, Taxus, Ulmus, Prunus, Quercus, Malus, Arbutus, Salix, Platanus, Acer, Tsuga, Pseudotsuga, Pinus, Fraxinus, Eucalyptus, Acacia, Abrus, Cupressus, Fagus, Juniperus, Thuja and Canya.

73. The method according to claim 64, wherein said first tree species and said second tree species are independently selected from the group consisting of Populus trichocarpa, Populus deltoides, Populus tremuloides or hybrid thereof, wherein said first tree species is different from said second tree species.

74. The method according to claim 64, wherein said first species is selected from the group consisting of Populus trichcarpa (clone 93-968), Populus deltoides (clone ILL-129), or hybrid thereof.

75. The method according to claim 64, wherein said first tree species is a pure species.

76. The method according to claim 64, wherein said first tree species is a hybrid.

77. The method according to claim 76, wherein said hybrid is a hybrid poplar.

78. The method according to claim 77, wherein said hybrid poplar is Populus trichocarpa X Populus deltoides—Family 331.

79. The method according to claim 64, wherein said identified markers are selected from the group consisting of I17_{—}04, G02_{—}11, E01_{—}04, P1027, P757, I14_{—}09, F15_{—}10, B15, H19_{—}08, G13_{—}17, P1054, P1018, H12, H07_{—}10 and G03.

80. The method according to claim 64, wherein said genetic markers represent candidate genes or are highly linked to candidate genes.

81. The method according to claim 64, wherein said genetic markers are physically and/or genetically linked to candidate genes.

82. The method according to claim 64, wherein said first genus and/or species and/or hybrid thereof is naturally or artificially produced.

83. The method according to claim 64, wherein said second genus and/or species and/or hybrid thereof is naturally or artificially produced.

84. The method according to claim 64, wherein DNA for identifying said genetic markers is obtained from a leaf, cotyledon, cambium, root, bud, stem, cork, phloem, flower or xylem.

85. The method of claim 64, wherein said parent tree is the seed parent tree to each of said progeny trees.

86. A stand of vegetatively produced enhanced property trees produced by the method of claim 64, the genome of said trees containing the same genetic marker associated with a genetic locus conferring at least one enhanced property in said second tree of a different genus and/or species than said first parent tree.

87. A genetic map of QTLs of trees associated with enhanced properties as set forth in FIG. 30.

88. The genetic map of claim 21, wherein said enhanced properties are selected from the group consisting of fiber length, fiber coarseness, DBH (diameter at breast height), microfibril angle, density, pulp strength, pulp yield, lignin content, pitch propensity, air resistance, kraft pulping H-factor, specific refining energy, wood extractive compounds content and calcium accumulation.

89. The genetic map of claim 43 wherein said enhanced properties are selected from the group consisting of fiber length, fiber coarseness, DBH (diameter at breast height), microfibril angle, density, pulp strength, pulp yield, lignin content, pitch propensity, air resistance, kraft pulping H-factor, specific refining energy, wood extractive compounds content and calcium accumulation.

90. The genetic map of claim 66, wherein said enhanced properties are selected from the group consisting of fiber length, fiber coarseness, DBH (diameter at breast height), microfibril angle, density, pulp strength, pulp yield, lignin content, pitch propensity, air resistance, kraft pulping H-factor, specific refining energy, wood extractive compounds content and calcium accumulation.
